SEQUENCE LISTING (89 sequences)

SEQ ID NO: 1 - protein, 5 aa, Synthetic construct
HYYWS

SEQ ID NO: 2 - protein, 16 aa, synthetic construct
RIYTSGSTNYNPSLKS

SEQ ID NO: 3 - protein, 9 aa, synthetic construct
DRYTGYFDL

SEQ ID NO: 4 - protein, 12 aa, synthetic construct
RASQSVSSSYLA

SEQ ID NO: 5 - protein, 7 aa, synthetic construct
GASSRAT

SEQ ID NO: 6 - protein, 9 aa, synthetic construct
QQYGSSPPT

SEQ ID NO: 7 - protein, 117 aa, synthetic construct
QVQLQESGPGLVKPSETLSLTCTVSGASISHYYWSWIRRPAGKGLEWIGRIYTSGSTNYNPSLKSRVTVSVDTSKNQFSLKLSSVTAADTAVYYCARDRYTGYFDLWGRGTLVTVSS

SEQ ID NO: 8 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTFGGGTKLEIK

SEQ ID NO: 9 - protein, 447 aa, synthetic construct
QVQLQESGPGLVKPSETLSLTCTVSGASISHYYWSWIRRPAGKGLEWIGRIYTSGSTNYNPSLKSRVTVSVDTSKNQFSLKLSSVTAADTAVYYCARDRYTGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 10 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
RSTMH

SEQ ID NO: 12 - protein, 17 aa, synthetic construct
YINPSSAYTNYNQKFQG

SEQ ID NO: 13 - protein, 12 aa, synthetic construct
PQVHYDYAGFPY

SEQ ID NO: 14 - protein, 10 aa, synthetic construct
SASSSVSYMN

SEQ ID NO: 15 - protein, 7 aa, synthetic construct
DSSKLAS

SEQ ID NO: 16 - protein, 9 aa, synthetic construct
QQWSRNPPT

SEQ ID NO: 17 - protein, 121 aa, synthetic construct
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSS

SEQ ID NO: 18 - protein, 106 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIK

SEQ ID NO: 19 - protein, 477 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIKGGGSGGSGGCPPCGGSGGQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 1072 aa, Homo sapiens
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 21 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 22 - protein, 245 aa, synthetic construct
EIVLTQSPATLSASPGERVTLSCSASSSVSYMNWYQQKPGCAPRRWIYDSSKLASGVPARFSGSGSGRDYTLTISSLEPEDFAVYYCQQWSRNPPTFGGGTKVEIKGGGSGGSGGCPPCGGSGGQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGCGTLVTVSS

SEQ ID NO: 23 - protein, 7 aa, synthetic construct
GASISHY

SEQ ID NO: 24 - protein, 5 aa, synthetic construct
YTSGS

SEQ ID NO: 25 - protein, 10 aa, synthetic construct
GASISHYYWS

SEQ ID NO: 26 - protein, 9 aa, synthetic construct
RIYTSGSTN

SEQ ID NO: 27 - protein, 6 aa, synthetic construct
SHYYWS

SEQ ID NO: 28 - protein, 12 aa, synthetic construct
WIGRIYTSGSTN

SEQ ID NO: 29 - protein, 8 aa, synthetic construct
SSSYLAWY

SEQ ID NO: 30 - protein, 10 aa, synthetic construct
LLIYGASSRA

SEQ ID NO: 31 - protein, 8 aa, synthetic construct
QQYGSSPP

SEQ ID NO: 32 - DNA, 351 bp, synthetic construct
  source 1..351: mol_type = other DNA
caggtgcagctgcaggagtcgggcccaggactggtgaagccttcggagaccctgtccctcacctgcactgtctctggtgcctccatcagtcattactactggagctggatccggcggcccgccgggaagggactggagtggattgggcgtatctataccagtgggagcaccaactacaacccctccctcaagagtcgagtcaccgtgtcagtagacacgtccaagaaccagttctccctgaagctgagctctgtgaccgccgcggacacggccgtatattactgtgcgagagatcgatatacggggtacttcgatctctggggccgtggcaccctggtcactgtctcctca

SEQ ID NO: 33 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtttattactgtcagcagtatggtagctcacctcccactttcggcggagggaccaagctggagatcaaa

SEQ ID NO: 34 - DNA, 363 bp, synthetic construct
  source 1..363: mol_type = other DNA
caagtacaacttgttcaatctggcgctgaagtcaaaaaacccggatctagtgttaaagtttcatgtaaggcatcaggctatactttcactcggtctacaatgcactgggtcaagcaggctccagggcaaggtttggaatggatagggtacataaatccttctagcgcctatactaattacaaccagaagtttcagggacgtgttactctgactgctgacaaatcaacatctacagcctatatggaactgtcttcattgaggagtgaggacactgctgtgtattactgcgcttctcctcaggtgcactatgattacgctggctttccctactggggttgtggcaccctcgtcaccgtttcatct

SEQ ID NO: 35 - DNA, 318 bp, synthetic construct
  source 1..318: mol_type = other DNA
gagatcgtcttgactcagtcccctgcaaccctcagcgcaagtcccggagagcgcgttacattgagttgttccgcatccagttcagtcagctacatgaactggtaccaacaaaagcccggttgtgctccaagaaggtggatctatgattccagtaagctggcctccggggtcccagcaaggttcagcggttccggcagcggaagggactacactctgaccataagttcccttgaaccagaagacttcgccgtctattattgtcagcagtggtcccgaaatccacctaccttcgggggcggcactaaagtggagattaaa

SEQ ID NO: 36 - DNA, 735 bp, synthetic construct
  source 1..735: mol_type = other DNA
gagatcgtcttgactcagtcccctgcaaccctcagcgcaagtcccggagagcgcgttacattgagttgttccgcatccagttcagtcagctacatgaactggtaccaacaaaagcccggttgtgctccaagaaggtggatctatgattccagtaagctggcctccggggtcccagcaaggttcagcggttccggcagcggaagggactacactctgaccataagttcccttgaaccagaagacttcgccgtctattattgtcagcagtggtcccgaaatccacctaccttcgggggcggcactaaagtggagattaaaggaggaggtagtggcggctccggtggatgtcctccttgcggaggatcaggaggtcaagtacaacttgttcaatctggcgctgaagtcaaaaaacccggatctagtgttaaagtttcatgtaaggcatcaggctatactttcactcggtctacaatgcactgggtcaagcaggctccagggcaaggtttggaatggatagggtacataaatccttctagcgcctatactaattacaaccagaagtttcagggacgtgttactctgactgctgacaaatcaacatctacagcctatatggaactgtcttcattgaggagtgaggacactgctgtgtattactgcgcttctcctcaggtgcactatgattacgctggctttccctactggggttgtggcaccctcgtcaccgtttcatct

SEQ ID NO: 37 - DNA, 1341 bp, synthetic construct
  source 1..1341: mol_type = other DNA
caggtgcagctgcaggagtcgggcccaggactggtgaagccttcggagaccctgtccctcacctgcactgtctctggtgcctccatcagtcattactactggagctggatccggcggcccgccgggaagggactggagtggattgggcgtatctataccagtgggagcaccaactacaacccctccctcaagagtcgagtcaccgtgtcagtagacacgtccaagaaccagttctccctgaagctgagctctgtgaccgccgcggacacggccgtatattactgtgcgagagatcgatatacggggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagtctagatggcagcaggggaacgtcttctcatgctccgtgatgcatgaggctctgcacaaccggttcacgcagaagagcctctccctgtctccgggtaaa

SEQ ID NO: 38 - DNA, 645 bp, synthetic construct
  source 1..645: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtttattactgtcagcagtatggtagctcacctcccactttcggcggagggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 39 - DNA, 1431 bp, synthetic construct
  source 1..1431: mol_type = other DNA
gagatcgtcttgactcagtcccctgcaaccctcagcgcaagtcccggagagcgcgttacattgagttgttccgcatccagttcagtcagctacatgaactggtaccaacaaaagcccggttgtgctccaagaaggtggatctatgattccagtaagctggcctccggggtcccagcaaggttcagcggttccggcagcggaagggactacactctgaccataagttcccttgaaccagaagacttcgccgtctattattgtcagcagtggtcccgaaatccacctaccttcgggggcggcactaaagtggagattaaaggaggaggtagtggcggctccggtggatgtcctccttgcggaggatcaggaggtcaagtacaacttgttcaatctggcgctgaagtcaaaaaacccggatctagtgttaaagtttcatgtaaggcatcaggctatactttcactcggtctacaatgcactgggtcaagcaggctccagggcaaggtttggaatggatagggtacataaatccttctagcgcctatactaattacaaccagaagtttcagggacgtgttactctgactgctgacaaatcaacatctacagcctatatggaactgtcttcattgaggagtgaggacactgctgtgtattactgcgcttctcctcaggtgcactatgattacgctggctttccctactggggttgtggcaccctcgtcaccgtttcatctgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 40 - protein, 18 aa, synthetic construct
GGGSGGSGGCPPCGGSGG

SEQ ID NO: 41 - protein, 16 aa, synthetic construct
GGGSGGCPPCGGGSGG

SEQ ID NO: 42 - protein, 16 aa, synthetic construct
GGSGGSGGCPPCGSGG

SEQ ID NO: 43 - protein, 17 aa, synthetic construct
GGGSGGSGGCPPCGSGG

SEQ ID NO: 44 - protein, 17 aa, synthetic construct
GGGSGGGSGCPPCGGGG

SEQ ID NO: 45 - protein, 20 aa, synthetic construct
GGSEGKSSGSGSESKSTGGS

SEQ ID NO: 46 - protein, 8 aa, synthetic construct
GGGSGGGS

SEQ ID NO: 47 - protein, 12 aa, synthetic construct
GGGSGGGSGGGS

SEQ ID NO: 48 - protein, 16 aa, synthetic construct
GGGSGGGSGGGSGGGS

SEQ ID NO: 49 - protein, 20 aa, synthetic construct
GGGSGGGSGGGSGGGSGGGS

SEQ ID NO: 50 - protein, 15 aa, synthetic construct
GGGGSGGGGSGGGGS

SEQ ID NO: 51 - protein, 20 aa, synthetic construct
GGGGSGGGGSGGGGSGGGGS

SEQ ID NO: 52 - protein, 25 aa, synthetic construct
GGGGSGGGGSGGGGSGGGGSGGGGS

SEQ ID NO: 53 - protein, 18 aa, synthetic construct
GSTSGSGKPGSGEGSTKG

SEQ ID NO: 54 - protein, 14 aa, synthetic construct
IRPRAIGGSKPRVA

SEQ ID NO: 55 - protein, 15 aa, synthetic construct
GKGGSGKGGSGKGGS

SEQ ID NO: 56 - protein, 15 aa, synthetic construct
GGKGSGGKGSGGKGS

SEQ ID NO: 57 - protein, 15 aa, synthetic construct
GGGKSGGGKSGGGKS

SEQ ID NO: 58 - protein, 15 aa, synthetic construct
GKGKSGKGKSGKGKS

SEQ ID NO: 59 - protein, 15 aa, synthetic construct
GGGKSGGKGSGKGGS

SEQ ID NO: 60 - protein, 15 aa, synthetic construct
GKPGSGKPGSGKPGS

SEQ ID NO: 61 - protein, 20 aa, synthetic construct
GKPGSGKPGSGKPGSGKPGS

SEQ ID NO: 62 - protein, 20 aa, synthetic construct
GKGKSGKGKSGKGKSGKGKS

SEQ ID NO: 63 - protein, 14 aa, synthetic construct
STAGDTHLGGEDFD

SEQ ID NO: 64 - protein, 15 aa, synthetic construct
GEGGSGEGGSGEGGS

SEQ ID NO: 65 - protein, 15 aa, synthetic construct
GGEGSGGEGSGGEGS

SEQ ID NO: 66 - protein, 15 aa, synthetic construct
GEGESGEGESGEGES

SEQ ID NO: 67 - protein, 15 aa, synthetic construct
GGGESGGEGSGEGGS

SEQ ID NO: 68 - protein, 20 aa, synthetic construct
GEGESGEGESGEGESGEGES

SEQ ID NO: 69 - protein, 18 aa, synthetic construct
GSTSGSGKPGSGEGSTKG

SEQ ID NO: 70 - protein, 19 aa, synthetic construct
PRGASKSGSASQTGSAPGS

SEQ ID NO: 71 - protein, 19 aa, synthetic construct
GTAAAGAGAAGGAAAGAAG

SEQ ID NO: 72 - protein, 19 aa, synthetic construct
GTSGSSGSGSGGSGSGGGG

SEQ ID NO: 73 - protein, 20 aa, synthetic construct
GKPGSGKPGSGKPGSGKPGS

SEQ ID NO: 74 - protein, 4 aa, synthetic construct
GSGS

SEQ ID NO: 75 - protein, 10 aa, synthetic construct
APAPAPAPAP

SEQ ID NO: 76 - protein, 20 aa, synthetic construct
APAPAPAPAPAPAPAPAPAP

SEQ ID NO: 77 - protein, 32 aa, synthetic construct
AEAAAKEAAAKEAAAAKEAAAAKEAAAAKAAA

SEQ ID NO: 78 - protein, 16 aa, synthetic construct
GTEGKSSGSGSESKST

SEQ ID NO: 79 - protein, 10 aa, synthetic construct
ARDRYTGYFD

SEQ ID NO: 80 - protein, 8 aa, synthetic construct
GASISHYY

SEQ ID NO: 81 - protein, 7 aa, synthetic construct
IYTSGST

SEQ ID NO: 82 - protein, 11 aa, synthetic construct
ARDRYTGYFDL

SEQ ID NO: 83 - protein, 7 aa, synthetic construct
QSVSSSY

SEQ ID NO: 84 - protein, 5 aa, synthetic construct
TYAMN

SEQ ID NO: 85 - protein, 19 aa, synthetic construct
RIRSKYNNYATYYAASVKG

SEQ ID NO: 86 - protein, 14 aa, synthetic construct
HGNFGNSYVSWFAY

SEQ ID NO: 87 - protein, 14 aa, synthetic construct
RSSTGAVTTSNYAN

SEQ ID NO: 88 - protein, 7 aa, synthetic construct
GTNKRAP

SEQ ID NO: 89 - protein, 9 aa, synthetic construct
ALWYSNLWV

(19) United States
(12) Patent Application Publication
(10) Pub. No.: US 2025/0066510 A1
(43) Pub. Date: Feb. 27, 2025

(54) GUCY2C ANTIBODIES AND USES THEREOF

(71) Applicant: Janssen Biotech, Inc., Horsham, PA (US)

(72) Inventors: Jason Ho, Collegeville, PA (US); Lijs Beke, Antwerpen (BE); Jinquan Luo, Malvern, PA (US); Elisabeth Prinslow, Harleysville, PA (US); Adam Zwolak, Bala Cynwyd, PA (US); Cholpon Tilegenova, Horsham, PA (US)

(21) Appl. No.: 18/795,538

(22) Filed: Aug. 6, 2024

Related U.S. Application Data
(60) Provisional application No. 63/518,041, filed on Aug. 7, 2023.
(60) Provisional application No. 63/612,000, filed on Dec. 19, 2023.

(51) Int. Cl. C07K 16/40; A61P 35/00; C07K 16/28

(52) U.S. Cl. CPC C07K 16/40; A61P 35/00; C07K 16/2809; C07K 2317/31; C07K 2317/52; C07K 2317/55; C07K 2317/565; C07K 2317/622; C07K 2317/92; C07K 2317/94

(57) ABSTRACT

Disclosed herein are antibodies or antigen binding fragments thereof that bind guanylyl cyclase C (GUCY2C), multi-specific antibodies comprising the same, and methods of treating cancer using the same.

### CROSS-REFERENCE TO RELATED APPLICATIONS

This application claims priority to U.S. Provisional Application No. 63/518,041, filed on Aug. 7, 2023, and U.S. Provisional Application No. 63/612,000, filed on Dec. 19, 2023, the disclosures of each of which are hereby incorporated by reference in their entireties.

### SEQUENCE LISTING

The instant application contains a Sequence Listing which has been submitted electronically in XML format and is hereby incorporated by reference in its entirety. The XML copy, created on Jul. 18, 2024, is named JBI6829USNP1-SL.xml and is 85 KB in size.

### TECHNICAL FIELD

Provided are antibodies or antigen binding fragments thereof that bind Guanylate Cyclase 2C (GUCY2C), multi-specific antibodies comprising the same, and methods of treating cancer using the same.

### BACKGROUND

Gastroesophageal and pancreatic cancers are among the malignancies with the highest unmet medical needs (Devesa S S. et al, Cancer 1988; 83:2049-53). Gastric cancer is the second leading cause of cancer death worldwide (Karami P, Cancer Epidemiol Biomarkers Prev. 2014, 23(5): 700-713). Esophageal cancer incidence has increased in recent decades. In 2002, ˜4 million people worldwide developed gastroesophageal cancers and 1.1 million died (Kamangar, F. et al, J Clin Oncol 2006; 24(14):2137-50). The overall 5-year survival rates for gastroesophageal cancers are 20% to 25% despite the aggressiveness of established standard treatments associated with substantial side effects (Sahin U, Clin Cancer Res 2008: 14 (23), 7624-7634). For pancreatic cancer, there is an even greater medical need. Due to the advanced state of the disease at the time of diagnosis, the prognosis is extremely poor, with median survival times of less than 6 months and a 5-year survival rate of <5.5%.

Solid tumors have been a challenge for antibody-mediated T-cell redirection due to lack of cancer-specific targets leading to toxicities occurring at sub-efficacious doses. Unmet need therefore remains high across solid tumors to prolong and improve patient outcomes and overall treatment duration. Today, even with the best therapeutic options available, the prognosis for advanced gastric cancer is poor. Only 68% of patients receive first-line therapy for their disease. Efforts to stratify gastric/Gastroesophageal (GEJ) patients have been attempted but produced less than desirable impact. The need for more improved treatment and effective therapies for patients with gastric/GEJ cancer remains high.

Colorectal cancer (CRC) is the fourth leading cause of cancer, and the second leading cause of cancer-related death in the United States and world. While surgical excision of primary tumors can be curative, particularly at the earliest stages of disease, about 50% of patients with colorectal cancer ultimately die of distant metastases. While chemo-, radio-, and targeted therapies extend survival to about 24 months, less than 15% of patients with metastatic CRC survive beyond 5 years, highlighting the unmet need for new therapeutic paradigms for this disease.

Guanylate cyclase 2C (GUCY2C), also known as guanylyl cyclase C (GC-C), intestinal guanylate cyclase, guanylate cyclase-C receptor, or the heat-stable enterotoxin receptor (hSTAR) is an enzyme found in the luminal aspect of intestinal epithelium and dopamine neurons in the brain (GenBank Accession No. NM-004963 and GenPept Accession No. NP-004954). The receptor has an extracellular ligand-binding domain, a single transmembrane region, a region with sequence similar to that of protein kinases, and a C-terminal guanylate cyclase domain. GUCY2C's cell-surface expression is confined to the apical surfaces of intestinal epithelial cells and exhibits limited expression in extra-intestinal tissues of humans and mice. Of significance, GUCY2C is a cancer mucosa antigen, universally overexpressed by primary and metastatic human colorectal cancer and is ectopically expressed in esophageal and gastric cancers associated with intestinal dysplasia. Moreover, anatomical segregation of GUCY2C on the luminal surface of the intestinal epithelium limits access to systemically delivered GUCY2C-targeted molecules permitting diagnostic imaging and monoclonal antibody-based therapy of colorectal cancer metastasis without recognition of intestinal epithelium.

### SUMMARY

Provided herein is an isolated antibody or antigen binding fragment thereof that binds to GUCY2C comprising the heavy chain complementarity determining region 1 (HCDR1), the HCDR2 and the HCDR3 of a VH1 comprising the amino acid sequence of SEQ ID NO: 7; and the heavy chain complementarity determining region 1 (LCDR1), the LCDR2 and the LCDR3 of a VL1 comprising the amino acid sequence of SEQ ID NO: 8; wherein the HCDR1, HCDR2, HCDR3, LCDR1, LCDR2, and LCDR3 amino acid sequences preferably are according to the Kabat numbering system, the Chothia numbering system, the AbM numbering system, the IMGT numbering system or the Contact numbering system or a combination thereof, preferably according to the Kabat numbering system, the Chothia numbering system, the AbM numbering system, the Contact numbering system or the IMGT numbering system.

The disclosure also provides an isolated antibody or antigen binding fragment thereof, comprising the HCDR1, HCDR2, HCDR3, LCDR1, LCDR2 and LCDR3, wherein the HCDR1, HCDR2, HCDR3, LCDR1, LCDR2 and LCDR3 comprise the amino acid sequences of SEQ ID NOs: 1, 2, 3, 4, 5, and 6, respectively; and wherein the antibody or antigen binding fragment thereof binds GUCY2C.

The disclosure also provides an isolated antibody or antigen binding fragment thereof that binds GUCY2C, comprising a VH1 which is at least 80% (e.g. at least 85%, at least 90%, at least 95%, at least 99% or 100%) identical to the VH1 of SEQ ID NO: 7 and a VL1 which is at least 80% (e.g. at least 85%, at least 90%, at least 95%, at least 99% or 100%) identical to the VL1 of SEQ ID NO: 8.

The disclosure also provides an antibody or antigen binding fragment thereof that binds GUCY2C comprising an amino acid sequence at least 80%, at least 85%, at least 90%, at least 95%, at least 99% or 100% identical to the amino acid sequence of SEQ ID NO: 9 and 10.

The disclosure also provides a GUCY2CxCD3 antibody or antigen binding fragment comprising a first antigen binding domain that binds GUCY2C and a second antigen binding domain that binds CD3.

In some embodiments, the GUCY2CxCD3 antibody or antigen binding fragment thereof comprises a first binding domain that binds to GUCY2C, wherein the first binding domain comprises the HCDR1, the HCDR2 and the HCDR3 of a VH1 comprising an amino acid sequence of SEQ ID NO: 7; the LCDR1, the LCDR2, the LCDR3, of a VL1 comprising an amino acid sequence of SEQ ID NO: 8; wherein the HCDR1, HCDR2, HCDR3, LCDR1, LCDR2, and LCDR3 amino acid sequences are according to the Kabat numbering system, the Chothia numbering system, the AbM numbering system, the IMGT numbering system, the Contact numbering system or a combination thereof.

In some embodiments, the GUCY2CxCD3 antibody or antigen binding fragment thereof comprises a first binding domain that binds to GUCY2C comprising a heavy chain complementarity determining region 1 (HCDR1), a HCDR2, and a HCDR3, and a light chain complementarity determining region 1 (LCDR1), a LCDR2, and LCDR3 of SEQ ID NOs: 1, 2, 3, 4, 5, and 6, respectively.

In some embodiments, the GUCY2CxCD3 antibody or antigen binding fragment thereof comprises a first binding domain that binds GUCY2C comprising a VH1 and a VL1 comprising the amino acid sequence of SEQ ID NO: 7 and 8, respectively.

In some embodiments, the GUCY2CxCD3 antibody or antigen binding fragment thereof comprises a first binding domain that binds GUCY2C and a second binding domain that specifically binds CD3, wherein the first binding domain that binds GUCY2C comprises a heavy chain variable domain (VH) and a light chain variable domain (VL) of SEQ ID NO: 7 and 8 respectively, and wherein the second binding domain that binds CD3 comprises a spFv of SEQ ID NO: 22.

In some embodiments, the GUCY2CxCD3 antibody or antigen binding fragment thereof comprises a first binding domain that binds GUCY2C and a second binding domain that specifically binds CD3, wherein the first binding domain that binds GUCY2C comprises a heavy chain (HC1) and a light chain (LC1) of SEQ ID NO: 9 and 10 respectively, and wherein the second binding domain that binds CD3 comprises a heavy chain (HC2) of SEQ ID NO: 19.

The disclosure provides methods of killing cancer cells overexpressing GUCY2C and/or method of treating a GUCY2C expressing cancer, comprising administering to the subject an GUCY2CxCD3 antibody, or antigen binding fragment thereof that comprises a first binding domain that binds GUCY2C and a second binding domain that binds CD3, wherein the first binding domain that binds GUCY2C comprises
(i) the HCDR1, HCDR2, HCDR3 of a VH1 comprising an amino acid sequence of SEQ ID NO: 7; and the LCDR1, LCDR2 and LCDR3 of a VL1 comprising an amino acid sequence of SEQ ID NO: 8;
(ii) a VH1 comprising a HCDR1, a HCDR2, and a HCDR3 having an amino acid sequence of SEQ ID NOs: 1, 2, and 3, respectively, and a VL1 comprising a LCDR1, LCDR2, and LCDR3 having an amino acid sequence of SEQ ID NOs: 3, 4, and 5 respectively; wherein the HCDR1, HCDR2, HCDR3, LCDR1, LCDR2, and LCDR3 amino acid sequences preferably are according to the Kabat numbering system, the Chothia numbering system, the AbM numbering system, the Contact numbering system or the IMGT numbering system or a combination thereof, preferably according to the Kabat numbering system, the Chothia numbering system, the AbM numbering system, the Contact numbering system or the IMGT numbering system
(iii) a VH1 having an amino acid sequence of SEQ ID NO: 7 and a VL1 having an amino acid sequence of SEQ ID NO: 8; and/or
(iv) a HC1 having an amino acid sequence of SEQ ID NO: 9 and a LC1 having an amino acid sequence of SEQ ID NO: 10; and
the second binding domain that binds CD3 comprises
(i) a spFv having an amino acid sequence of SEQ ID NO: 22; or
(ii) a HC2 having an amino acid sequence of SEQ ID NO: 19.

### DESCRIPTION OF DRAWINGS

FIG. 1 shows binding of GUCY2C_mAb to human and cyno GUCY2C.

FIG. 2A shows binding of GUCY2CxCD3_biAb-1 and 2 to T84 cell line.

FIG. 2B shows binding of GUCY2CxCD3_biAb-1 and 2 to T cells.

FIG. 3 shows competition flow cytometry between GUCY2C_biAb-1 and reference biAb.

FIG. 4A, FIG. 4B, and FIG. 4C show T cell mediated cytotoxicity of GUCY2CxCD3_biAb-1, GUCY2CxCD3_biAb-2 and reference biAb. FIG. 4D, FIG. 4E, and FIG. 4F show T-cell activation of GUCY2CxCD3_biAb-1, GUCY2CxCD3 biAb-2 and reference biAb.

FIG. 5 shows anti-tumor efficacy of GUCY2CxCD3_biAb-1 in a T-cell humanized HT55 CDX model. Abbreviations: TGI, Tumor Growth Inhibition; #CR, number of complete response; #PR, number of partial response.

FIG. 6 shows T cell-mediated cytotoxic activity of GUCY2CxCD3_biAb-1 on WT and CRC tumor organoids. The upper panel shows live cell fluorescence images showing apoptosis (white) of CRC and WT organoids post treatment with GUCY2CxCD3 biAb-1 and reference biAb. The lower panel shows dose normalized cytotoxicity dose response curves of GUCY2CxCD3 biAb-1 and reference biAb. Abbreviations: CRC, colorectal cancer; EpCAM, epithelial cell adhesion molecule; HC, high control; LC, low control; WT, wild type.

### DETAILED DESCRIPTION OF THE INVENTION

Provided herein are isolated anti-GUCY2C antibodies or antigen-binding fragments thereof, polynucleotides and expression vectors encoding the antibodies, host cells containing the vectors, and compositions comprising the antibodies. Methods of making the antibodies, and methods of using the antibodies to treat diseases are also provided.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

The disclosed isolated anti-GUCY2C antibody, GUCY2CxCD3 bi-specific antibody, antigen binding fragment thereof, polynucleotides, vectors, cells, compositions, kits, and methods may be understood more readily by reference to the following detailed description which forms a part of this disclosure. It is to be understood that the disclosed antibodies, antigen binding domains, antibody fragments, polynucleotides, vectors, cells, compositions, kits, and methods are not limited to those specifically described and/or shown herein, and that the terminology used herein is for the purpose of describing particular embodiments by way of example only and is not intended to be limiting of the claimed antibody, antigen binding domains, antibody fragments, polynucleotides, vectors, cells, compositions, kits, and methods.

Unless specifically stated otherwise, any description as to a possible mechanism or mode of action or reason for improvement is meant to be illustrative only, and the disclosed antibodies, antigen binding fragments thereof, polynucleotides, vectors, cells, compositions, kits, and methods are not to be constrained by the correctness or incorrectness of any such suggested mechanism or mode of action or reason for improvement.

Throughout this text, where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

“About” as used herein when referring to a measurable value is meant to encompass variations that are reasonably close to the specified value. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed disclosure. Embodiments described in terms of the phrase “comprising” (or its equivalents) also provide as embodiments those independently described in terms of “consisting of” and “consisting essentially of” Embodiments described in terms of the phrase “consisting essentially of” (or its equivalents) also provide as embodiments those independently described in terms of “consisting of.”

As used in this specification and the appended claims, the phrase “and fragments thereof” when appended to a list includes fragments of one or more members of the associated list. The list may comprise a Markush group so that, as an example, the phrase “the group consisting of peptides A, B, and C, and fragments thereof” specifies or recites a Markush group including A, B, C, fragments of A, fragments of B, and/or fragments of C.

All publications, including but not limited to patents and patent applications, cited in this specification are herein incorporated by reference as though fully set forth.

Any reference in the description to a method of treatment (comprising the administration of a compound, composition, medicament, etc.) equally refers to the compound, pharmaceutical composition, medicament, etc. for use in the method for treatment or for use in the manufacture of the medicament. For example, every embodiment or combination of embodiments, which has been described herein for methods for treating a disease, disorder or condition (comprising the administration of a compound, composition, medicament, combination, association etc.), is also applicable to the same compound, composition, medicament, combination, association, etc. for use in treating the same disease, disorder or condition as well as to the use of the same compound, composition, medicament, combination, association, etc. in the manufacture of a medicament for the treatment of the same disease, disorder or condition.

In an attempt to help the reader of the application, the description has been separated in various paragraphs or sections or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

Antibodies that Bind GUCY2C

Disclosed herein are antibodies that bind to GUCY2C.

The antibodies disclosed herein possess one or more desirable functional properties, including but not limited to high-affinity binding to GUCY2C or high specificity to GUCY2C. In certain embodiments, the antibodies disclosed herein possess the ability to treat or prevent a disease or disorder when administered to a subject alone or in combination with other therapies.

As used herein, the term “GUCY2C” refers to guanylyl cyclase C (GC-C), a member of the guanylyl cyclase (GC; heat-stable enterotoxin receptor [STaR]) family of enzymes that convert intracellular guanosine triphosphate (GTP) to cyclic guanosine monophosphate (cGMP) upon ligand binding. GUCY2C is a 120 kDa transmembrane protein expressed on the luminal surface of epithelial cells in the intestine. It plays a role in gut fluid secretion, tumor suppression, inflammation, and intestine homeostasis though cGMP-regulated mechanisms. Dysregulation of the GUCY2C axis (through mutation or enterotoxigenic bacterial infection) results in fluid secretion disorders.

Unless specified, as used herein, “GUCY2C” refers to human GUCY2C. The amino acid sequence of full length human GUCY2C (Accession #P25092.2) is shown in SEQ ID NO: 20. The extracellular domain of human GUCY2C spans from amino acid residue 24 amino acid residue 430, which is followed by a transmembrane domain (amino acid residue 431-454) and a cytoplasmic domain (amino acid residue 455-1-73).

(full-length human GUCY2C)
SEQ ID NO: 20
MKTLLLDLALWSLLFQPGWLSFSSQVSQNCHNGSYEISVLMMGNSA
FAEPLKNLEDAVNEGLEIVRGRLQNAGLNVTVNATFMYSDGLIHN
SGDCRSSTCEGLDLLRKISNAQRMGCVLIGPSCTYSTFQMYLDTE
LSYPMISAGSFGLSCDYKETLTRLMSPARKLMYFLVNFWKTNDLP
FKTYSWSTSYVYKNGTETEDCFWYLNALEASVSYFSHELGFKVVL
RQDKEFQDILMDHNRKSNVIIMCGGPEFLYKLKGDRAVAEDIVII
LVDLFNDQYFEDNVTAPDYMKNVLVLTLSPGNSLLNSSFSRNLSP
TKRDFALAYLNGILLFGHMLKIFLENGENITTPKFAHAFRNLTFE
GYDGPVTLDDWGDVDSTMVLLYTSVDTKKYKVLLTYDTHVNKTYP
VDMSPTFTWKNSKLPNDITGRGPQILMIAVFTLTGAVVLLLLVAL
LMLRKYRKDYELRQKKWSHIPPENIFPLETNETNHVSLKIDDDKR
RDTIQRLRQCKYDKKRVILKDLKHNDGNFTEKQKIELNKLLQIDY
YNLTKFYGTVKLDTMIFGVIEYCERGSLREVLNDTISYPDGTFMD
WEFKISVLYDIAKGMSYLHSSKTEVHGRLKSTNCVVDSRMVVKIT
DFGCNSILPPKKDLWTAPEHLRQANISQKGDVYSYGIIAQEIILR
KETFYTLSCRDRNEKIFRVENSNGMKPFRPDLFLETAEEKELEVY
LLVKNCWEEDPEKRPDFKKIETTLAKIFGLFHDQKNESYMDTLIR
RLQLYSRNLEHLVEERTQLYKAERDRADRLNFMLLPRLVVKSLKE
KGFVEPELYEEVTIYFSDIVGFTTICYSTPMEVVDMLNDIYKSFD
HIVDHHDVYKVETIGDAYMVASGLPKRNGNRHAIDIAKMALEILS
FMGTFELEHLPGLPIWIRIGVHSGPCAAGVVGIKMPRYCLFGDTV
NTASRMESTGLPLRIHVSGSTIAILKRTECQFLYEVRGETYLKGR
GNETTYWLTGMKDQKFNLPTPPTVENQQRLQAEFSDMIANSLQKR
QAAGIRSQKPRRVASYKKGTLEYLQLNTTDKESTYF

As used herein the term “Antibody” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, dimeric, tetrameric, multimeric or biparatopic antibodies, single chain antibodies, domain antibodies and any other modified configuration of the immunoglobulin molecule that comprises an antigen binding site of the required specificity. The term antibody includes full length antibodies, whole antibodies, intact antibodies, antibody fragments, antigen binding fragment and antigen binding domains.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. In some embodiments, the antibodies of the invention are IgG1, IgG2, IgG3 or IgG4.

The Ig constant region allotype is related to amino acid sequence variations at specific locations in the constant region sequences of the antibody. Table 1 shows selected IgG1, IgG2 and IgG4 allotypes.

TABLE 1

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 1 | 282 | 309 | 422 | 214 | 356 | 358 | 431 |
| G2m(n) | T | M | | | | | | |
| G2m(n−) | P | V | | | | | | |
| G2m(n)/(n−) | T | V | | | | | | |
| nG4m(a) | | | L | R | | | | |
| Glm(17) | | | | | K | E | M | A |
| Glm(17, 1) | | | | | K | D | L | A |

Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain.

As used herein, HC1 and LC1 refer to the heavy chain and the light chain, respectively, of the binding domain that binds GUCY2C. As used herein, HC2 refer to the heavy chain of the binding domain that binds CD3.

In addition to the heavy and light constant domains, antibodies contain an antigen-binding region that is made up of a light chain variable region and a heavy chain variable region, each of which contains three domains i.e., “complementarity determining regions” (CDRs) 1-3; CDR1, CDR2, and CDR3. The light chain variable region domains are alternatively referred to as LCDR1, LCDR2, and LCDR3, and the heavy chain variable region domains are alternatively referred to as HCDR1, HCDR2, and HCDR3. Throughout the present application, the CDRs are given in according Kabat numbering system, unless otherwise specified.

Complementarity determining regions (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton (1996) J Mol Biol 263: 800-15). The correspondence between the various delineations and variable region numbering is described (Honegger and Pluckthun (2001), J Mol Biol 309:657-70; International ImMunoGeneTics (IMGT) database; Web resources, www_imgt_org). Available programs such as abYsis by UCL Business PLC may be used to delineate CDRs. The terms “CDR”, “HCDR1”, “HCDR2”, “HCDR3”, “LCDR1”, “LCDR2” and “LCDR3” as used herein include CDRs defined by any of the standardized numbering methods known in the art to define complementary determining regions such as, but not limited to, Kabat, Chothia, IMGT, Contact or AbM, unless the numbering method is otherwise explicitly stated in the specification. Correspondence between the numbering system, including, for example, the Kabat numbering and the IMGT unique numbering system, is well known to one skilled in the art. Correspondence between the IMGT, Kabat, AbM, Chothia and Contact numbering system is shown in Table 2.

TABLE 2
Correspondence between the different numbering system

| | IMGT | Kabat | AbM | Chothia | Contact |
|---|---|---|---|---|---|
| HCDR1 | 27-38 | 31-35 | 26-35 | 26-32 | 30-35 |
| HCDR2 | 56-65 | 50-65 | 50-58 | 52-56 | 47-58 |
| HCDR3 | 105-117 | 95-102 | 95-102 | 95-102 | 93-102 |
| LCDR1 | 27-38 | 24-34 | 24-34 | 24-35 | 24-34 |
| LCDR2 | 56-65 | 50-56 | 50-56 | 50-56 | 50-56 |
| LCDR3 | 105-117 | 89-97 | 89-97 | 89-97 | 89-97 |

The term “variable region” or “variable domain” refers to the heavy or light chain domain that is involved in the binding of the antibody to the antigen. The variable domains of the heavy or light chain (VH and VL, respectively) comprise four framework regions (FR) and the three complementarity determining regions (CDRs). As used herein, VH1 and VL1 refer to the heavy chain variable domain and the light chain variable domain, respectively, of the binding domain that binds GUCY2C. As used herein, VH2 and VL2 refer to the heavy chain variable domain and the light chain variable domain, respectively, of the binding domain that binds CD3.

As used herein, the term “isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

As used herein, the term an “isolated antibody” refers to an antibody which is substantially free of other antibodies having different antigenic specificities (e.g., an isolated antibody that specifically binds to GUCY2C is substantially free of antibodies that do not bind to GUCY2C). In addition, an isolated antibody is substantially free of other cellular material and/or chemicals. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

The terms “anti-GUCY2C antibody” and “GUCY2C antibody” are used interchangeably and refer to an antibody of antigen binding domain that specially binds GUCY2C.

Within the present disclosure, the terms “Specifically binds,” “specific binding,” “specifically binding”, and “binds” are used interchangeably and refer to a proteinaceous molecule binding to an antigen or an epitope within the antigen with greater affinity than for other antigens.

“Epitope” refers to a portion of an antigen to which an antibody specifically binds. Epitopes typically consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

The binding affinity of an antibody or fragment to a protein can be measured with many methods known in the art and can be expressed as KD or EC50. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

The binding affinity can also be expressed as half maximal effective concentration (EC50) which is the concentration of antibody at which 50% maximal binding to cells overexpressing the antigen occurs. The EC50 of an antibody can be determined using methods well known in the art. In some embodiments, the antibody or antigen binding fragment thereof that binds GUCY2C binds to GUCY2C with an EC50 of 100 nM or less, 50 nM or less, 10 nM or less, 5 nM or less, 1 nM or less. The EC50 may be 10 nM or less, 5 nM or less, 2 nM or less, or 1 nM or less. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less, 0.01 nM or less, 0.001 nM or less.

In some embodiments, the EC50 of the anti-GUCY2C antibody is about 5 nM or lower. In some embodiments, the EC50 of the anti-GUCY2C antibody is about 5 nM, about 4 nM, about 3 nM, about 2 nM, about 1 nM, about 0.5 nM, or lower.

In some embodiments, the isolated antibody or antigen binding fragment thereof that binds to GUCY2C comprises the heavy chain complementarity determining region 1 (HCDR1), the HCDR2 and the HCDR3 of a VH1 comprising the amino acid sequence of SEQ ID NO: 7; and the light chain complementarity determining region 1 (LCDR1), the LCDR2 and the LCDR3 of a VL1 comprising the amino acid sequence of SEQ ID NO: 8.

In some embodiments, the isolated antibody or antigen binding fragment thereof that binds to GUCY2C comprises the heavy chain complementarity determining region 1 (HCDR1), the HCDR2 and the HCDR3 of a VH1 comprising the amino acid sequence of SEQ ID NO: 7; and the light chain complementarity determining region 1 (LCDR1), the LCDR2 and the LCDR3 of a VL2 comprising the amino acid sequence of SEQ ID NO: 8; wherein the HCDR1, HCDR2, HCDR3, LCDR1, LCDR2, and LCDR3 amino acid sequences are according to the Kabat numbering system, the Chothia numbering system, the AbM numbering system, the IMGT numbering system, the Contact numbering system or a combination thereof.

The disclosure also provides an isolated antibody or antigen binding fragment thereof that bind to GUCY2C, comprising a HCDR1, HCDR2, HCDR3, LCDR1, LCDR2 of:
a. SEQ ID NOs: 1, 2, 3, 4, 5, and 6, respectively;
b. SEQ ID NO: 23, 24, 3, 4, 5, and 6, respectively;
c. SEQ ID NO: 25, 26, 3, 4, 5, and 6, respectively;
d. SEQ ID NO: 27, 28, 79, 29, 30 and 32, respectively; or
e. SEQ ID NO: 80, 81, 82, 83, amino acid sequence GAS and SEQ ID NO: 6, respectively.

In some embodiments, the antibody or antigen binding fragment thereof that binds GUCY2C comprises the HCDR1, the HCDR2, the HCDR3, the LCDR1, the LCDR2, and the LCDR3 of SEQ ID NO: 1, 2, 3, 4, 5, and 6, respectively.

In one embodiment, the GUCY2C antibody or GUCY2C binding fragment thereof comprises a VH1 of SEQ ID NO: 7 and/or a VL1 of SEQ ID NO: 8.

In one embodiment, the GUCY2C antibody or GUCY2C-binding fragment thereof comprises a VH1 of SEQ ID NO: 7 and a VL1 or SEQ ID NO: 8.

In some embodiments, the isolated antibody or antigen binding fragment thereof that binds GUCY2C comprises a heavy chain (HC1) comprising the amino acid sequence of SEQ ID NO: 9 and a light chain (LC1) comprising the amino acid sequence of SEQ ID NO: 10.

Also disclosed is an isolated antibody or antigen binding fragment thereof comprising:
(i) the HCDRs of a VH1 comprising an amino acid sequence of SEQ ID NO: 7; and the LCDRs of a VL1 comprising an amino acid sequence of SEQ ID NO: 8;
(ii) a VH1 comprising a HCDR1, a HCDR2, and a HCDR3 having an amino acid sequence of SEQ ID NOs: 1, 2, and 3, respectively, and (ii) a VL1 comprising a LCDR1, LCDR2, and LCDR3 having an amino acid sequence of SEQ ID NOs: 4, 5, and 6, respectively; wherein the HCDR1, HCDR2, HCDR3, LCDR1, LCDR2, and LCDR3 amino acid sequences preferably are according to the Kabat numbering system, the Chothia numbering system, the AbM numbering system, the Contact numbering system or the IMGT numbering system or a combination thereof, preferably according to the Kabat numbering system, the Chothia numbering system, the AbM numbering system, the Contact numbering system or the IMGT numbering system
(iii) a VH1 having an amino acid sequence of SEQ ID NO: 7; and (ii) a VL1 having an amino acid sequence of SEQ ID NO: 8; and/or
(iv) a HC1 having an amino acid sequence of SEQ ID NO: 9; and (ii) a LC1 having an amino acid sequence of SEQ ID NO: 10;
and wherein the isolated antibody or antigen binding fragment thereof binds GUCY2C.
   Multispecific Antibodies that Bind GUCY2C and CD3

The disclosure also provides multispecific antibodies that bind GUCY2C.

In some embodiments, the multispecific antibody comprises a first binding domain that binds to GUCY2C, and a second binding domain that binds to a second target that is not GUCY2C. In some embodiment, the multispecific antibody is a bispecific antibody comprising a first binding domain that binds to GUCY2C, and a second binding domain that binds to a second target that is not GUCY2C.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., different proteins (or different subunits of a multimeric protein). In an embodiment, a multispecific antibody comprises a third, fourth or fifth immunoglobulin variable domain. In an embodiment, a multispecific antibody is a bispecific antibody molecule, a trispecific antibody molecule, or a tetraspecific antibody molecule.

In some embodiment, the multispecific antibody comprises a first binding domain that binds to GUCY2C and one or more other binding domains that bind to other antigens.

In some embodiments, the first binding domain comprises or is the GUCY2C antibody or GUCY2C-binding fragment thereof, as disclosed above. In some embodiment, the multispecific antibody comprises a first binding domain that binds to GUCY2C, and a second binding domain that binds to a second target that is an antigen on lymphocytes. In some embodiments, the antigens on lymphocyte are selected from antigens on T cells. In some embodiments, the antigens on lymphocyte are selected from antigens on CD8+ T cells. In some embodiments the antigens on lymphocyte are selected from antigens on natural killer (NK) cells. In some embodiments, the antigens on lymphocyte are selected from CD3, CD8, KI2L4, NKG2E, NKG2D, NKG2F, BTNL3, CD186, BTNL8, PD-1, CD195, and NKG2C. In some embodiment, the antigen on lymphocyte is CD3 epsilon (CD38).

In some embodiments, the multispecific antibodies comprises a first binding domain comprising or being the GUCY2C antibody or GUCY2C-binding fragment thereof, as disclosed above, and a second binding domain that binds to CD3, wherein the second binding domain is a CD3 antibody or CD3-binding fragment thereof.

In some embodiments, the multispecific antibody is a bispecific antibody comprising the GUCY2C antibody or GUCY2C-binding fragment thereof, as disclosed above, and a CD3 antibody or CD3-binding fragment thereof.

As used herein, the term “bispecific antibody” refers to a multispecific antibody that binds no more than two epitopes or two antigens. “Bispecific antibody” refers to an antibody that specifically binds two distinct antigens or two distinct epitopes within the same antigen. A bispecific antibody is characterized by a first immunoglobulin variable domain sequence which has binding specificity for a first epitope (e.g., an epitope on a GUCY2C antigen) and a second immunoglobulin variable domain sequence that has binding specificity for a second epitope. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

The term “CD3” refers to an antigen that is expressed on T cells as part of the multimeric T cell receptor (TCR) complex and which consists of a homodimer or heterodimer formed from the association of two or four receptor chains: CD3 epsilon, CD3 delta, CD3 zeta and CD3 gamma. In some embodiments, CD3 antibodies provided herein bind to the CD3-epsilon polypeptide, which together with CD3-gamma, -delta and -zeta, and the T cell receptor alpha/beta and gamma/delta heterodimers, forms the T cell receptor-CD3 complex. This complex plays an important role in coupling antigen recognition to several intracellular signal-transduction pathways. The CD3 complex mediates signal transduction, resulting in T cell activation and proliferation. CD3 is required for the immune response. The term “CD3” includes any CD3 variant, isoform, and species homolog, which is naturally expressed by cells (including T cells) or can be expressed on cells transfected with genes or cDNA encoding the polypeptide. In specific embodiments, the CD3 is a human CD3. All references to proteins, polypeptides and protein fragments herein are intended to refer to the human version of the respective protein, polypeptide or protein fragment unless explicitly specified as being from a non-human species. Thus, “CD3” means human CD3 unless specified as being from a non-human species, e.g., “mouse CD3” “monkey CD3,” etc.

An exemplary human CD3 epsilon comprises the amino acid sequence of SEQ ID NO: 21
SEQ ID NO: 21 (human CD3 epsilon) MQSGTHWRVLGLCLLSVGVWGQDGNEEMGGITQTPYKVSISGTTVILTCPQYPGSE ILWQHNDKNIGGDEDDKNIGSDEDHLSLKEFSELEQSGYYVCYPRGSKPEDANFYL YLRARVCENCMEMDVMSVATIVIVDICITGGLLLLVYYWSKNRKAKAKPVTRGAG AGGRQRGQNKERPPPVPNPDYEPIRKGQRDLYSGLNQRRI

As used herein, an antibody that “binds to CD3” or that “specifically binds to CD3” refers to an antibody that binds to CD3, preferably human CD3, with a K_{D} of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M, 5×10⁻¹¹ M, 1×10⁻¹¹ M, 5×10⁻¹² M, or 1×10⁻¹² M or less.

The binding affinity of an antibody or antibody fragment to an antigen can also be expressed as half maximal effective concentration (EC50) which is the concentration of antibody at which 50% maximal binding to cells overexpressing the antigen occurs. An antibody that binds CD3 refers to an antibody that binds CD3 with an EC50 of 100 nM or less, 50 nM or less, 10 nM or less, 5 nM or less, 1 nM or less. The EC50 may be 10 nM or less, 5 nM or less, 2 nM or less, or 1 nM or less. The K_{D} may be 0.9 nM or less, 0.8 nM or less, 0.7 nM or less, 0.6 nM or less, 0.5 nM or less, 0.4 nM or less, 0.3 nM or less, 0.2 nM or less, or 0.1 nM or less, 0.01 nM or less, 0.001 nM or less.

In some embodiments, the CD3 antibody or CD3-binding fragment disclosed herein binds to CD3 with an EC50 of about 50-200 nM, or about 60-180 nM, or about 70-170 nM, or about 75-160 nM.

As used herein, “GUCY2CxCD3 antibody”, GUCY2CxCD3 bispecific antibody”, and “GUCY2CxCD3 biAb” refer to a bispecific antibody with a first binding domain that binds GUCY2C and a second binding domain that binds CD3. The domains specifically binding GUCY2C are typically referred herein as VH1/VL1 pairs. The domains specifically binding CD3 are typically referred herein as VH2/VL2 pairs. The heavy chain and light chain of the binding domain specifically binding GUCY2C are referred herein as HC1 and LC1, respectively. The heavy chain of the binding domain specifically binding CD3 is referred herein as HC2.

In some embodiments, the GUCY2CxCD3 antibody or antigen binding fragment thereof comprises a first binding domain that binds to GUCY2C, wherein the first binding domain comprises the HCDR1, the HCDR2 and the HCDR3 of a VH1 comprising an amino acid sequence of SEQ ID NO: 7; the LCDR1, the LCDR2, the LCDR3, of a VL1 comprising an amino acid sequence of SEQ ID NO: 8; wherein the HCDR1, HCDR2, HCDR3, LCDR1, LCDR2, and LCDR3 amino acid sequences preferably are according to the Kabat numbering system, the Chothia numbering system, the AbM numbering system, the Contact numbering system or the IMGT numbering system or a combination thereof, preferably according to the Kabat numbering system, the Chothia numbering system, the AbM numbering system, the Contact numbering system or the IMGT numbering system.

In some embodiments, the GUCY2CxCD3 antibody or antigen binding fragment thereof comprises a first binding domain that binds to GUCY2C comprising a heavy chain complementarity determining region 1 (HCDR1), a HCDR2, and a HCDR3, and a light chain complementarity determining region 1 (LCDR1), a LCDR2, and LCDR3 of SEQ ID NOs: 1, 2, 3, 4, 5, and 6, respectively.

In some embodiments, the GUCY2CxCD3 antibody or antigen binding fragment thereof comprises a first binding domain that binds GUCY2C comprising a VH1 and a VL1 comprising the amino acid sequence of SEQ ID NO: 7 and 8, respectively;

In some embodiments, the GUCY2CxCD3 antibody or antigen binding fragment thereof comprises a first binding domain that binds GUCY2C, wherein the first binding domain that binds GUCY2C comprises a first heavy chain (HC1) and a first light chain (LC1) comprising the amino acid sequence of SEQ ID NO: 9 and 10, respectively;

In some embodiments, the GUCY2CxCD3 antibody or antigen binding fragment thereof comprises a second binding domain that binds CD3.

In some embodiments, the GUCY2CxCD3 antibody or antigen binding fragment thereof comprises a first binding domain that binds GUCY2C and a second binding domain that binds CD3, wherein the second binding domain that binds CD3 comprises the HCDR1, HCDR2, HCDR3, LCDR1, LCDR2 and LCDR3 of SEQ ID NO: 11, 12, 13, 14, 15 or 16, respectively.

In some embodiments, the GUCY2CxCD3 antibody or antigen binding fragment thereof comprises a first binding domain that binds GUCY2C and a second binding domain that binds CD3, wherein the second binding domain that binds CD3 comprises a heavy chain variable domain (VH2) having an amino acid sequence of SEQ ID NOs: 17 and a light chain variable domain (VL2) having an amino acid sequence of SEQ ID NOs: 18.

In some embodiments, the GUCY2CxCD3 antibody or antigen binding fragment thereof comprises a first binding domain that binds GUCY2C and a second binding domain that binds CD3, wherein the second binding domain that binds CD3 comprises a heavy chain variable domain (VH2) and a light chain variable domain (VL2) having an amino acid sequence of SEQ ID NOs: 17 and 18, respectively.

### Forms of the GUCY2C Antibodies and GUCY2CxCD3 Antibodies

### Intact Antibodies and Antibody Fragments

The anti-GUC2C antibodies and the GUCY2CxCD3 antibodies described herein can have any form as long as they retain their binding functions to GUCY2C and/or CD3. In some embodiments, anti-GUCY2C antibody or the GUCY2CxCD3 antibody include intact antibodies or antibody fragments such as Fv fragments, single chain scFv fragments (scFv), single chain disulfide bond stabilized scFv fragments or stapled scFv fragment (spFv), Fab, F(ab)₂, or single chain antibodies.

The term “intact antibodies” refers to an antibody having a structure similar to a native antibody. “Intact antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). Antibody light chains of any vertebrate species may be assigned to one of two clearly distinct types, namely kappa (κ) and lambda (λ), based on the amino acid sequences of their constant domains. The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

As used herein, the term “antibody fragment” refers to a molecule other than an intact antibody. Antibody fragments may be synthetic, enzymatically obtainable or genetically engineered polypeptides and include portions of an immunoglobulin that bind an antigen, such as a VH, a VL, a VH and a VL, a Fab, a Fab′, a F(ab′)2, a Fd and a Fv fragments, a disulfide stabilized Fv fragment (dsFv), a (dsFv)2, a bispecific dsFv (dsFv-dsFv′), a disulfide stabilized diabody (ds diabody), a single-chain antibody molecule (scFv), or a disulfide stabilized single-chain antibody molecule (spFv or stapled scFv), a single domain antibody (sdab) an scFv dimer (bivalent diabody), a multispecific antibody formed from a portion of an antibody comprising one or more CDRs, a camelized single domain antibody, a nanobody, a domain antibody, a domain antibody (dAb) consisting of one VH domain or one VL domain, a shark variable IgNAR domain, a camelized VH domain, a VHH domain, a minimal recognition unit consisting of the amino acid residues that mimic the CDRs of an antibody, such as a FR3-CDR3-FR4 portion, the HCDR1, the HCDR2 and/or the HCDR3 and the LCDR1, the LCDR2 and/or the LCDR3, an alternative scaffold that bind an antigen, a bivalent domain antibody, a multispecific protein comprising the antibody or any other antibody fragment that binds to an antigen but does not comprise a complete antibody structure.

In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a disulfide stabilized single-chain antibody molecule (spFv). In some embodiments, the antigen binding fragment is a single domain antibody (sdAb). In some embodiments, the antigen binding fragment is an scFv dimer (bivalent diabody). In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is an antibody fragment that binds to an antigen but does not comprise a complete antibody structure.

“Diabodies” are bivalent dimers formed from two chains, each containing a VH and a VL domain. The two domains within a chain are separated by a linker that is too short to facilitate intrachain dimerization leading to two chains dimerizing in a head-to-tail arrangement. The linker may be a pentameric glycine-rich linker.

“VHH” refers to a single-domain antibody or nanobody, exclusively composed of the antigen binding domain of a heavy chain. A VHH single domain antibody lacks the light chain and the CH1 domain of the heavy chain of conventional Fab region.

“Fab” or “Fab fragment” refers to an antibody fragment composed of VH, CH1, VL and CL domains.

“F(ab′)2” or “F(ab′)2 fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody. Fv fragments lack the constant regions of Fab (CH1 and CL) regions. The VH and VL in Fv fragments are held together by non-covalent interactions.

Antigen binding fragments (such as VH and VL) may be linked together via a synthetic linker to form various types of single antibody designs where the VH/VL domains may be paired intramolecularly, or intermolecularly to form a monovalent antigen binding domain, such as single chain Fv (scFv) or diabody. “Single chain Fv” or “scFv” are fusion proteins comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. A scFv may have the VL and VH variable regions in either order, e.g., with respect to the N-terminal and C-terminal ends of the polypeptide, the scFv may comprise VL-linker-VH or may comprise VH-linker-VL.

Divalent or bivalent single chain variable fragments (di-scFv, bi-scFvs) can be engineered by linking two scFvs“. (scFv)2” or “tandem scFv” or “bis-scFv” fragments refers to a fusion protein comprising two light chain variable regions (VL) and two heavy chain variable regions (VH), wherein the two VL and the two VH regions are contiguously linked via polypeptide linkers, and capable of being expressed as a single chain polypeptide. The two VL and two VH regions fused by peptide linkers form a bivalent molecule VLA-linker-VHA-linker-VLB-linker-VHB to form two binding sites, capable of binding two different antigens or epitopes concurrently. (ScFv)2 can be expressed as a single chain polypeptide.

Any of the VH and the VL domains identified herein that bind GUCY2C or CD3 may be engineered into scFv format in either VH-linker-VL or VL-linker-VH orientation. Any of the VH and the VL domains identified herein may also be used to generate sc(Fv)2 structures, such as VH-linker-VL-linker-VL-linker-VH, VH-linker-VL-linker-VH-linker-VL, VH-linker-VH-linker-VL-linker-VL,VL-linker-VH-linker-VH-linker-VL,VL-linker-VH-linker-VL-linker-VH or VL-linker-VL-linker-VH-linker-VH.

Any of the VH and the VL domains identified herein (e.g. those that bind GUCY2C or those that bind CD3) may be engineered into scFv format in either VH-linker-VL or VL-linker-VH orientation. In some embodiments, the scFv format is, from the N- to C-terminus, in the VH-linker-VL orientation. In other embodiments, the scFv format is, from the N- to C-terminus, in the VL-linker-VH orientation.

Any of the VH and the VL domains identified herein can also be used to generate sc(Fv)₂ structures. In some embodiments, the sc(Fv)₂ structure is VH-linker-VL-linker-VL-linker-VH. In some embodiments, the sc(Fv)₂ structure is VH-linker-VL-linker-VH-linker-VL. In some embodiments, the sc(Fv)₂ structure is VH-linker-VH-linker-VL-linker-VL. In some embodiments, the sc(Fv)₂ structure is VL-linker-VH-linker-VH-linker-VL. In some embodiments, the sc(Fv)₂ structure is VL-linker-VH-linker-VL-linker-VH.

An scFv can comprise a linker peptide, such as two to about eight glycine or other amino acid residues, which connects the variable region of the heavy chain and the variable region of the light chain. In some embodiments, the linker is a peptide linker. In some embodiments, the liker comprises a naturally occurring amino acid. Exemplary amino acids that can be included into the linker are Gly, Ser Pro, Thr, Glu, Lys, Arg, Ile, Leu, His and Phe. In some embodiments, the linker has a length that is adequate to link the VH and the VL in such a way that they form the correct conformation relative to one another so that they retain the desired activity, such as binding to the target (e.g., GUCY2C or CD3).

In some embodiments, the linker is about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long.

Exemplary linkers that can be used are Gly rich linkers, Gly and Ser containing linkers, Gly and Ala containing linkers, Ala and Ser containing linkers, and other flexible linkers.

Exemplary linkers that can be used include any one of SEQ ID NOs 45-78. Exemplary linkers are shown in Table 3. Additional linkers are described for example in Int. Pat. Publ. No. WO2019/060695. Other linker sequences may include portions of immunoglobulin hinge area, CL or CH1 derived from any immunoglobulin heavy or light chain isotype. Alternatively, a variety of non-proteinaceous polymers, including polyethylene glycol (PEG), polypropylene glycol, polyoxyalkylenes, or copolymers of polyethylene glycol and polypropylene glycol, may find use as linkers.

TABLE 3
The amino acid sequences of linkers.

| | | SEQ |
|---|---|---|
| Linker | | ID |
| name | Amino acid sequence | NO: |
| Linker 1 | GGSEGKSSGSGSESKSTGGS | 45 |
| Linker 2 | GGGSGGGS | 46 |
| Linker 3 | GGGSGGGSGGGS | 47 |
| Linker 4 | GGGSGGGSGGGSGGGS | 48 |
| Linker 5 | GGGSGGGSGGGSGGGSGGGS | 49 |
| Linker 6 | GGGGSGGGGSGGGGS | 50 |
| Linker 7 | GGGGSGGGGSGGGGSGGGGS | 51 |
| Linker 8 | GGGGSGGGGSGGGGSGGGGSGGGGS | 52 |
| Linker 9 | GSTSGSGKPGSGEGSTKG | 53 |
| Linker 10 | IRPRAIGGSKPRVA | 54 |
| Linker 11 | GKGGSGKGGSGKGGS | 55 |
| Linker 12 | GGKGSGGKGSGGKGS | 56 |
| Linker 13 | GGGKSGGGKSGGGKS | 57 |
| Linker 14 | GKGKSGKGKSGKGKS | 58 |
| Linker 15 | GGGKSGGKGSGKGGS | 59 |
| Linker 16 | GKPGSGKPGSGKPGS | 60 |
| Linker 17 | GKPGSGKPGSGKPGSGKPGS | 61 |
| Linker 18 | GKGKSGKGKSGKGKSGKGKS | 62 |
| Linker 19 | STAGDTHLGGEDFD | 63 |
| Linker 20 | GEGGSGEGGSGEGGS | 64 |
| Linker 21 | GGEGSGGEGSGGEGS | 65 |
| Linker 22 | GEGESGEGESGEGES | 66 |
| Linker 23 | GGGESGGEGSGEGGS | 67 |
| Linker 24 | GEGESGEGESGEGESGEGES | 68 |
| Linker 25 | GSTSGSGKPGSGEGSTKG | 69 |
| Linker 26 | PRGASKSGSASQTGSAPGS | 70 |
| Linker 27 | GTAAAGAGAAGGAAAGAAG | 71 |
| Linker 28 | GTSGSSGSGSGGSGSGGGG | 72 |
| Linker 29 | GKPGSGKPGSGKPGSGKPGS | 73 |
| Linker 30 | GSGS | 74 |
| Linker 31 | APAPAPAPAP | 75 |
| Linker 32 | APAPAPAPAPAPAPAPAPAP | 76 |
| Linker 33 | AEAAAKEAAAKEAAAAKEAAAAKEAAAAKAAA | 77 |
| Linker 34 | GTEGKSSGSGSESKST | 78 |

### Stapled Antibodies

In some embodiments, the scFv may be stabilized by the introduction of two disulfide bonds between the linker and the variable domains of the scFv. The generation of stabilized scFv molecules (herein referred to as spFv (or stapled scFv) is described in WO2021/030657 filed Aug. 14, 2020, which is incorporated herein by reference in its entirety.

“Stapled single chain Fv”, “stapled scFv” or “spFv” refers to a scFv that comprises one or more disulfide bonds between the VH and the linker or the VL and the linker. Typically, the spFv may comprise one disulfide bond between the VH and the linker, and one disulfide bond between the VL and the linker, or two disulfide bonds between the VH and the linker and the VL and the linker.

“Staple” refers to the spFv linker that contains one or two Cys residues which that are capable of forming a disulfide bond with the anchor point Cys. Exemplary linkers that contain two Cys residues are listed in Table 4.

TABLE 4
spFv linkers with two Cys residues.

| Linker amino acid sequence | SEQ ID NO |
|---|---|
| GGGSGGSGGCPPCGGSGG | 40 |
| GGGSGGCPPCGGGSGG | 41 |
| GGSGGSGGCPPCGSGG | 42 |
| GGGSGGSGGCPPCGSGG | 43 |
| GGGSGGGSGCPPCGGGG | 44 |

As used herein, “anchor point” refers to a VH or a VL framework residue in the scFv that can be mutated into Cys without adverse effect to the overall scFv structure and is capable of forming a disulfide bond with a Cys residing in the scFv linker.

As used herein, “VH Cysteine” or “VH Cys” refers to a Cys residue that resides in the VH framework and “VL Cysteine” or “VL Cys” refers to a Cys residue that resides in the VL framework.

The disclosure provides GUCY2CxCD3 antibodies or an antigen binding fragment thereof comprising a first binding domain that binds GUCY2C and a second binding domain that binds CD3, wherein the first binding domain or the second binding domain comprises a disulfide stabilized scFv (spFv).

In some embodiments, the GUCY2CxCD3 antibody comprises, consists of and/or consists essentially of a first variable domain that binds GUCY2C and a second variable domain that binds CD3, wherein the first variable domain that binds GUCY2C is a Fab and wherein the second variable domain that binds CD3 is a disulfide stabilized scFv (spFv) comprising, consisting of and/or consisting essentially of from the N to C-terminus, a VL comprising a VL Cysteine, a Linker of Table 4 and a VH comprising a VH Cysteine (VL-Linker-VH).

In some embodiments, the GUCY2CxCD3 antibody comprises, consists of and/or consists essentially of a first variable domain that binds GUCY2C and a second variable domain that binds CD3, wherein the first variable domain that binds GUCY2C is a Fab and wherein the second variable domain that binds CD3 is a disulfide stabilized scFv (spFv) comprising, consisting of and/or consisting essentially of from the N to C-terminus, a VH comprising, consisting of and/or consisting essentially of a VH Cysteine, a Linker of Table 4 and a VL comprising a VL Cysteine (VH-Linker-VL).

In some embodiments, the spFv comprises a first disulfide bond between a structurally conserved surface exposed VH position that is mutated to cysteine (Cys) and a first Linker Cys; and a second disulfide bond between a structurally conserved surface exposed VL position that is mutated to Cys and a second Linker Cys.

In some embodiments, the VH Cys of the spFv is at position H3, H5, H40, H43, H46 or H105, wherein residue numbering is according to Chothia.

In some embodiments, the VL Cys of the spFv is at position L3, L5, L39, L42, L43, L45, L100 or L102, wherein residue numbering is according to Chothia.

In some embodiments, the VH Cys is at H105 and the VL Cys is at L42;
the VH Cys is at H43 and the VL Cys is at L100;
the VH Cys is at H3 and the VL Cys is at L3;
the VH Cys is at H3 and the VL Cys is at L5;
the VH Cys is at H3 and the VL Cys is at L39;
the VH Cys is at H3 and the VL Cys is at L42;
the VH Cys is at H3 and the VL Cys is at L45;
the VH Cys is at H3 and the VL Cys is at L100;
the VH Cys is at H3 and the VL Cys is at L102;
the VH Cys is at H5 and the VL Cys is at L3;
the VH Cys is at H5 and the VL Cys is at L5;
the VH Cys is at H5 and the VL Cys is at L39;
the VH Cys is at H5 and the VL Cys is at L42;
the VH Cys is at H5 and the VL Cys is at L45;
the VH Cys is at H5 and the VL Cys is at L100;
the VH Cys is at H5 and the VL Cys is at L102;
the VH Cys is at H40 and the VL Cys is at L3;
the VH Cys is at H40 and the VL Cys is at L5;
the VH Cys is at H40 and the VL Cys is at L39;
the VH Cys is at H40 and the VL Cys is at L42;
the VH Cys is at H40 and the VL Cys is at L45;
the VH Cys is at H40 and the VL Cys is at L100;
the VH Cys is at H40 and the VL Cys is at L102;
the VH Cys is at H43 and the VL Cys is at L3;
the VH Cys is at H43 and the VL Cys is at L5;
the VH Cys is at H43 and the VL Cys is at L39;
the VH Cys is at H43 and the VL Cys is at L42;
the VH Cys is at H43 and the VL Cys is at L45;
the VH Cys is at H43 and the VL Cys is at L102;
the VH Cys is at H46 and the VL Cys is at L3;
the VH Cys is at H46 and the VL Cys is at L5;
the VH Cys is at H46 and the VL Cys is at L39;
the VH Cys is at H46 and the VL Cys is at L42;
the VH Cys is at H46 and the VL Cys is at L45;
the VH Cys is at H46 and the VL Cys is at L100;
the VH Cys is at H46 and the VL Cys is at L102;
the VH Cys is at H105 and the VL Cys is at L3;
the VH Cys is at H105 and the VL Cys is at L5;
the VH Cys is at H105 and the VL Cys is at L39;
the VH Cys is at H105 and the VL Cys is at L43;
the VH Cys is at H105 and the VL Cys is at L45;
the VH Cys is at H105 and the VL Cys is at L100; or
the VH Cys is at H105 and the VL Cys is at L102, wherein residue numbering is according to Chothia.

In some embodiments, the spFv comprises a Linker from Table 4.

In some embodiments, the spFv Linker comprises, consists of and/or consists essentially of the amino acid sequence of SEQ ID NO: 40.

In some embodiments, the spFv Linker comprises, consists of and/or consists essentially of the amino acid sequence of SEQ ID NO: 41.

In some embodiments, the spFv Linker comprises, consists of and/or consists essentially of the amino acid sequence of SEQ ID NO: 42.

In some embodiments, the spFv Linker comprises, consists of and/or consists essentially of the amino acid sequence of SEQ ID NO: 43.

In some embodiments, the spFv Linker comprises, consists of and/or consists essentially of the amino acid sequence of SEQ ID NO: 44.

In some embodiments, the spFv linker is the linker of sequence GGGSGGSGGCPPCGGSGG (SEQ ID NO: 40).

In some embodiments, the spFv linker is the linker of sequence GGGSGGCPPCGGGSGG (SEQ ID NO: 41).

In some embodiments, the spFv linker is the linker of sequence GGSGGSGGCPPCGSGG (SEQ ID NO: 42).

In some embodiments, the spFv linker is the linker of sequence GGGSGGSGGCPPCGSGG (SEQ ID NO: 43).

In some embodiments, the spFv linker is the linker of sequence GGGSGGGSGCPPCGGGG (SEQ ID NO: 44).

In some embodiments, the spFv comprises a VH comprising a Cys at H105; a VL comprising a Cys at L42; and a Linker comprising an amino acid sequence of SEQ ID NOs: 40, 41, 42, 43 or 44; wherein the spFv is in the VL-Linker-VH orientation.

In some embodiments, the spFv comprises, consists of and/or consists essentially of a VH comprising, consisting of and/or consisting essentially of a Cys at H105; a VL comprising a Cys at L42; and a Linker comprising, consisting of and/or consisting essentially of an amino acid sequence of SEQ ID NO: 40; wherein the spFv is in the VL-Linker-VH orientation.

In some embodiments, the GUCY2CxCD3 antibody comprises a first variable domain that binds GUCY2C and a second variable domain that binds CD3, wherein the first variable domain that binds GUCY2C is a Fab and wherein the second variable domain that binds CD3 is a disulfide stabilized scFv (spFv) comprising from the N to C-terminus, a VL comprising a VL Cysteine at position L42, a Linker of Table 4 and a VH comprising a VH Cysteine at position H105 (VL-Linker-VH).

In some embodiments, the GUCY2CxCD3 antibody comprises a first variable domain that binds GUCY2C and a second variable domain that binds CD3, wherein the first variable domain that binds GUCY2C is a Fab and wherein the second variable domain that binds CD3 is a disulfide stabilized scFv (spFv) comprising from the N to C-terminus, a VL comprising a VL Cysteine at position L42, the Linker of SEQ ID NO: 40, and a VH comprising a VH Cysteine at position H105 (VL-Linker-VH).

In some embodiments, the GUCY2CxCD3 antibody or antigen binding fragment thereof comprises a first binding domain that binds GUCY2C and a second binding domain that binds CD3, wherein the second binding domain that binds CD3 comprises a spFv of SEQ ID NOs: 22.

In some embodiments, the GUCY2CxCD3 antibody or antigen binding fragment thereof comprises a first binding domain that binds GUCY2C and a second binding domain that binds CD3, wherein the second binding domain that binds CD3 comprises a spFv of SEQ ID NOs: 22.

The “stapling” strategy described herein is widely applicable to all VH/VL domains and pre-existing scFv molecules described herein.

While the specific examples disclose spFv with two disulfide bonds, it is readily envisioned that spFv with one disulfide bond, formed between the linker Cys and either the VH Cys or the VL Cys can be made and utilized, generating “half-anchored” molecules. The anchor positions are the same in spFv having one or two disulfide bonds. The linker Cys position may vary in the half-anchored molecule as long as it satisfies distance and geometry requirements for disulfide bond formation with the anchor point. It is expected that the half-anchored spFv will restrain VL/VH relative movement similar to the VL/VH pair stabilized with two disulfide bonds, and thus will also be stabilizing.

### FC Mutations

In addition to the modification set forth above, the anti-GUCY2C antibody, the GUCY2CxCD3 antibody, the antigen binding fragment thereof of the present disclosure and their variants may be conjugated to other antibodies, proteins, antigen binding fragments or alternative scaffolds that may be used to adjust, alter, improve or moderate antibody characteristics as desired. The constant region of the anti-GUCY2C Fab and/or anti-CD3 spFv of the GUCY2CxCD3 bispecific antibody may also comprises at least one mutation that may be used to adjust, alter, improve or moderate antibody characteristics as desired.

### Half-Life Mutations

For example, antibodies with increased in vivo half-lives can be generated by attaching half-life extending moiety such as albumin, albumin variants, albumin-binding proteins and/or domains, transferrin and fragments and analogues thereof, immunoglobulins (Ig) or fragments thereof, such as Fc regions to the antibody, antigen binding fragment of the disclosure. Additional half-life extending moieties include polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the antibody or antigen binding fragment of the disclosure and may be generated by standard cloning and expression techniques.

Half-life extending moieties can be attached to antibodies or antibody fragments or derivatives with or without a multifunctional linker either through conjugation to the N- or C-terminus of said antibodies or antibody fragments or via epsilon-amino groups present on lysine residues. A pegyl moiety may for example be conjugated to the anti-GUCY2C antibody, the GUCY2CxCD3 antibody or the antigen binding fragment by incorporating a cysteine residue to the C-terminus of the antibody or antigen binding fragment.

In some embodiments, the half-life extending moiety is albumin.

In some embodiments, the half-life extending moiety is the albumin binding domain.

In some embodiments, the half-life extending moiety is transferrin.

In some embodiments, the half-life extending moiety is polyethylene glycol.

In some embodiments, the half-life extending moiety is an Ig constant region or a fragment of the Ig constant region.

In some embodiments, the half-life extending moiety is an Ig.

In some embodiments, the half-life extending moiety is a fragment of the Ig.

In some embodiments, the half-life extending moiety is the Ig constant region.

In some embodiments, the half-life extending moiety is the fragment of the Ig constant region.

In some embodiments, the half-life extending moiety is the Fc region.

The Ig constant region or the fragment of the Ig constant region, such as the Fc region present in the antibody or antigen binding fragment thereof of the disclosure may be of any allotype or isotype, i.e., IgG1, IgG2, IgG3, IgG4, IgM, IgA and IgE. Ig constant region allotype is related to amino acid sequence variations at specific locations in the constant region sequences of the antibody.

In some embodiments, the Ig constant region or the fragment of the Ig constant region is an IgG1 isotype.

In some embodiments, the Ig constant region or the fragment of the Ig constant region is an IgG2 isotype.

In some embodiments, the Ig constant region or the fragment of the Ig constant region is an IgG3 isotype.

In some embodiments, the Ig constant region or the fragment of the Ig constant region is an IgG4 isotype.

In some embodiments the anti-GUCY2C antibody, the GUCY2CxCD3 antibody or the antigen binding fragment thereof is conjugated to an Ig constant region or the fragment of the Ig constant region comprising at least one mutation in the Ig constant region or in the fragment of the Ig constant region.

In some embodiments, the at least one mutation is in the Fc region.

The neonatal Fc receptor (FcRn) plays a central role in the cellular trafficking and serum half-life of IgGs. In some embodiments, the anti-GUCY2C antibody, the GUCY2CxCD3 antibody or the antigen binding fragment thereof is conjugated to an Ig constant region or to the fragment of the Ig constant region comprising at least one mutation in the Fc region that modulates binding of the anti-GUCY2C antibody, the GUCY2CxCD3 antibody or the antigen binding fragment thereof to FcRn and modulates the half-life of the antibody.

In some embodiments, the anti-GUCY2C antibody, the GUCY2CxCD3 antibody or the antigen binding fragment thereof is conjugated to an Ig constant region or to the fragment of the Ig constant region comprises at least one, two, three, four, five, six, seven, eight, nine, ten, eleven, twelve, thirteen, fourteen or fifteen mutations in the Fc region.

Fc positions that may be mutated to modulate half-life (e.g. binding to FcRn) include positions 250, 252, 253, 254, 256, 257, 307, 376, 380, 428, 434 and 435. Exemplary mutations that may be made singularly or in combination are mutations T250Q, M252Y, 1253A, S254T, T256E, P257I, T307A, D376V, E380A, M428L, H433K, N434S, N434A, N434H, N434F, H435A and H435R. Exemplary singular or combination mutations that may be made to increase the half-life are mutations M428L/N434S, M252Y/S254T/T256E, T250Q/M428L, N434A and T307A/E380A/N434A. In some embodiments, the at least one mutation that modulates the half-life of the antibody or antigen binding fragment thereof of the disclosure and their functional equivalents is selected from the group consisting of H435A, P257I/N434H, D376V/N434H, M252Y/S254T/T256E/H433K/N434F, T308P/N434A and H435R, wherein residue numbering is according to the EU index.

In some embodiments, the anti-GUCY2C antibody, the GUCY2CxCD3 antibody or the antigen binding fragment thereof is conjugated to an Ig constant region or to a fragment of an Ig constant region comprising the M252Y/S254T/T256E mutation.

### Fc Silencing Mutations

The anti-GUCY2C antibody, the GUCY2CxCD3 antibody or the antigen binding fragment thereof and their functional equivalents may also be conjugated to an Ig constant region or to the fragment of an Ig constant region that modulates the antibody effector functions such as ADCC, ADCP and/or ADCP and/or pharmacokinetic properties. This may be achieved by introducing mutation(s) into the Fc that modulate binding of the mutated Fc to activating FcγRs (FcγRI, FcγRIIa, FcγRIII), and/or to inhibitory FcγRIIb.

In some embodiments, the anti-GUCY2C antibody, the GUCY2CxCD3 antibody or the antigen binding fragment thereof is conjugated to an Ig constant region or to the fragment of the Ig constant region comprising at least one mutation in the Fc region that reduces binding of the protein to an activating Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP).

Fc positions that may be mutated to reduce binding to the activating FcγR and subsequently reduce effector function include positions 214, 233, 234, 235, 236, 237, 238, 265, 267, 268, 270, 295, 297, 309, 327, 328, 329, 330, 331 and 365. Exemplary mutations that may be made singularly or in combination are mutations K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination mutations that result in proteins with reduced ADCC are mutations L234A/L235A on IgG1, L234A/L235A/D265S on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4. Hybrid IgG2/4 Fc domains may also be used, such as Fc with residues 117-260 from IgG2 and residues 261-447 from IgG4.

In some embodiments, the anti-GUCY2C antibody, the GUCY2CxCD3 antibody or the antigen binding fragment is conjugated to the Ig constant region or to the fragment of the Ig constant region comprising L234A/L235A/D265S mutation.

### Dimerization Mutations

In addition, Fc mutation may include CH3 mutations that promote dimerization to generate the GUCY2CxCD3 antibodies of the disclosure and their variants.

The antigen binding regions that bind GUCY2C and the antigen binding domains that bind CD3 may be engineered to generate the GUCY2CxCD3 antibodies of the disclosure. Substitutions that promote heterodimerization may be introduced within the CH3 domain of the Ig constant region of GUCY2C and CD3 monospecific antibodies to promote Fab arm exchange in vitro. In the methods, two monospecific bivalent antibodies may be engineered to have certain substitutions at the CH3 domain that promote heterodimer stability; the antibodies may be incubated together under reducing conditions sufficient to allow the cysteines in the hinge region to undergo disulfide bond isomerization; thereby generating a bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of. 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed), the Strand Exchange Engineered Domain body (SEEDbody) (EMD Serono), Duobody® mutations (Genmab), and other asymmetric mutations (e.g., Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T3661_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

SEEDbody mutations involve substituting select IgG residues with IgA residues to promote heavy chain heterodimerization as described in US20070287170.

Other exemplary mutations that may be used are R409D_K370E/D399K_E357K, S354C_T366W/Y349C_T366S_L368A_Y407V, Y349C_T366W/S354C_T366S_L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E_D282K_K322D/D239K_E240K_K292D, K392D_K409D/D356K_D399K as described in WO2007/147901, WO 2011/143545, WO2013157954, WO2013096291 and US2018/0118849.

Duobody® mutations (Genmab) are disclosed for example in U.S. Pat. No. 9,150,663 and US2014/0303356 and include mutations F405L/K409R, wild-type/F405L_R409K, T350I_K370T_F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures into which the antigen binding regions that bind GUCY2C can be incorporated include Dual Variable Domain Immunoglobulins (DVD) (Int. Pat. Publ. No. WO2009/134776), structures that include various dimerization domains to connect the two antibody arms with different specificity, such as leucine zipper or collagen dimerization domains (Int. Pat. Publ. No. WO2012/022811; U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)₂-Fab (National Research Center for Antibody Medicine—China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

In some embodiments, the GUCY2CxCD3 antibody or the antigen binding fragment thereof is conjugated to an Ig constant region or to the fragment of an Ig constant region comprising a mutation that promotes dimerization.

In some embodiments, the GUCY2C2xCD3 antibody or the antigen binding fragment comprises the dimerization mutations T366W/T366S_L368A_Y407V.

In some embodiments, the GUC2CxCD3 antibody comprises a first binding domain comprising first heavy chain (HC1) and a first light chain (LC1), and a second binding domain comprising a second heavy chain (HC2), wherein the HC1 comprises the T366S, L368A, and Y407V mutations and the HC2 comprises the T366W mutation (numbering is according to EU index); or wherein the HC1 comprises the T366W mutation and the HC2 comprises the T366S, L368A, and Y407V mutations (numbering is according to EU index).

In some embodiments, the disclosure provides a GUCY2CxCD3 antibody or antigen binding fragment thereof, comprising a first binding domain that binds GUCY2C and a second binding domain that binds CD3, wherein
the first binding domain that binds GUCY2C comprises
(i) the HCDR1, HCDR2, HCDR3 of a VH1 comprising an amino acid sequence of SEQ ID NO: 7; and the LCDR1, LCDR2 and LCDR3 of a VL1 comprising an amino acid sequence of SEQ ID NO: 8; wherein the HCDR1, HCDR2, HCDR3, LCDR1, LCDR2, and LCDR3 amino acid sequences preferably are according to the Kabat numbering system, the Chothia numbering system, the AbM numbering system, the Contact numbering system or the IMGT numbering system or a combination thereof, preferably according to the Kabat numbering system, the Chothia numbering system, the AbM numbering system, the Contact numbering system or the IMGT numbering system;
(ii) a VH1 comprising a HCDR1, a HCDR2, and a HCDR3 having an amino acid sequence of SEQ ID NOs: 1. 2, and 3, respectively, and a VL1 comprising a LCDR1, LCDR2, and LCDR3 having an amino acid sequence of SEQ ID NOs: 3, 4, and 5 respectively;
(iii) a VH1 having an amino acid sequence of SEQ ID NO: 7 and a VL1 having an amino acid sequence of SEQ ID NO: 8; and/or
(iv) a HC1 having an amino acid sequence of SEQ ID NO: 9 and a LC1 having an amino acid sequence of SEQ ID NO: 10; and the second binding domain that binds CD3 comprises
(i) a spFv having an amino acid sequence of SEQ ID NO: 22; or
(ii) a HC2 having an amino acid sequence of SEQ ID NO: 19.

### Variants of the GUCY2C and GUCY2CxCD3 Antibodies

Variants of said antibody or antigen binding fragment thereof are also object of the disclosure. The antibodies of the present disclosure include variants of the disclosed antibody or antigen binding fragment thereof that bind GUCY2C, that include polypeptides with amino acid sequences substantially identical to the amino acid sequence of the variable domain or of hypervariable domain of the antibodies of the present disclosure or polypeptides with conservative substitutions. The variant antibodies of the disclosure have sufficient homology with the sequences of the antibody or antigen binding fragment of the disclosure and are functionally similar to the unmodified anti-GUCY2C antibody or GUCY2C xCD3 antibody to retain binding to GUCY2C or retain at least one of the activities of the unmodified antibody.

The term “variants” refer to antibodies comprising one or more mutations, substitutions, deletions and/or additions of one or more amino acid residues. Such an addition, substitution or deletion can be located at any position in the molecule. In the case where several amino acids have been added, substituted or deleted, any combination of addition, substitution or deletion can be considered, on condition that the resulting antibody still has at least the advantageous properties of the antibody of the disclosure.

In some embodiments, the antibodies and antigen binding fragments thereof provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may also include a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties.

In some embodiments, the disclosure provides amino acid sequence modification(s) of the antibodies or antigen binding fragment thereof described herein. For example, it may be desirable to improve the binding affinity and/or other biological properties of the antibody, including but not limited to specificity, thermostability, expression level, effector functions, glycosylation (e.g., fucosylation), reduced immunogenicity, or solubility. Thus, in addition to the antibodies and antigen binding fragment described herein, antibody variants can be prepared by introducing appropriate nucleotide changes into the encoding DNA, and/or by adding mutations, substitutions, deletions and/or additions of one or more amino acid residues to the antibodies and antigen binding fragment described herein.

The terms “identical” or percent “identity” in the context of two or more nucleic acids or polypeptide sequences (e.g., anti-GUCY2C antibodies and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection. The percent (%) amino acid sequence identity with respect to a reference polypeptide is defined as the percentage of amino acid residues in a given sequence that are identical to the amino acid residues in the reference polypeptide sequence. The percent (%) identity between the two sequences is a function of the number of identical positions shared by the sequences (i.e., % identity=number of identical positions/total number of positions ×100), taking into account the number of gaps, and the length of each gap, which need to be introduced for optimal alignment of the two sequences. The percent identity between two amino acid sequences may be determined using various the algorithms that are within the skill in the art, using publicly available software such as BLAS, BLAST-2, ALIGN. Megalin (DNASTAR) or the GAP program available in the GCG software package.

A polypeptide is typically substantially identical to a second polypeptide, for example, where the two peptides differ only by conservative substitutions. The antibodies of the present disclosure also include those for which binding characteristics, functional or physical properties have been improved by direct mutations. In some embodiments, variants of the antigen binding domains that bind GUCY2C or of the antigen binding domains that bind CD3 comprise one or two conservative substitutions in any of the CDR regions, while retaining desired functional properties of the parent antigen binding fragments.

Sequences of the disclosure may comprise amino acid sequences with at least 80% identity or homology to the sequences of the antibody or antigen binding fragment thereof, described above. In some embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% to the antigen binding domains that bind GUCY2C of the disclosure. Variants of the antigen binding domains comprising 1, 2, 3, 4, 5, 6, 7, 8, 9, 10, 11, 12, 13, 14, 15, 16, 17, 18, 19, 20, 21, 22, 23, 24, 25, 26, 27, 28 or 29 amino acid substitutions in the antigen binding domain that bind GUCY2C are within the scope of the disclosure, as long as they retain or have improved functional properties when compared to the parent antigen binding domains. Variations in the antigen binding domains that bind GUCY2C include one or more deletions and/or additions of one or more amino acid residues. Such an addition, substitution or deletion can be located at any position in the molecule. In the case where several amino acids have been added, substituted or deleted, any combination of addition, substitution or deletion can be considered, on condition that the resulting antibody still has at least the advantageous properties of the antibody of the disclosure.

In a specific embodiment, the substitution is a conservative amino acid substitution made at one or more predicted non-essential amino acid residues. “Conservative modifications” or “conservative substitution” refer to amino acid modifications that do not significantly affect or alter the binding characteristics of the antibody containing the amino acid modifications. Conservative modifications include amino acid substitutions, additions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine).

Standard techniques known to those of skill in the art can be used to introduce mutations in the nucleotide sequence encoding a molecule provided herein, including, for example, site-directed mutagenesis and PCR-mediated mutagenesis which results in amino acid substitutions.

Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed, and the activity of the protein can be determined.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

### Variants of GUCY2C Antibody

The disclosure provides variants of the GUCY2C antibody.

In some embodiments, the GUCY2C antibody or antigen binding fragment thereof comprises a VH1 which is at least 80% (e.g. at least 80%, at least 85%, at least 90%, at least 95%, at least 99% or 100%) identical to the VH1 of SEQ ID NO: 7 and a VL1 which is at least 80% (e.g. at least 80%, at least 85%, at least 90%, at least 95%, at least 99% or 100%) identical to the VL1 of SEQ ID NO: 8.

In one embodiment, the VH1 of the GUCY2C antibody or GUCY2C-biding fragment thereof may comprise an amino acid sequence about 90%, about 95%, about 98%, or about 99% identical to SEQ ID NO: 7.

In one embodiment, the VL1 of the GUCY2C antibody or GUCY2C-binding fragment thereof may comprise an amino acid sequence about 90%, about 95%, about 98%, or about 99% identical to SEQ ID NO: 8.

In some embodiments, the antibody or antigen binding fragment thereof that binds GUCY2C comprises a VH1 which is at least 80% (e.g. at least 80%, least 85%, at least 90%, at least 95%, at least 99% or 100%) identical to the VH1 of SEQ ID NO: 7 and a VL1 which is identical to the VL1 of SEQ ID NO: 8.

In some embodiments, the antibody or antigen binding fragment thereof that binds GUCY2C comprises a VH1 which is identical to the VH1 of SEQ ID NO: 7 and a VL1 which is at least 85% (e.g. at least 80%, least 85%, at least 90%, at least 95%, at least 99% or 100%) identical to the VL1 of SEQ ID NO: 8.

In some embodiments, the antibody or antigen binding fragment thereof that binds GUCY2C comprises a VH1 which is at least 95% identical to the VH1 of SEQ ID NO: 7 and a VL1 which is at least 95% identical to the VL1 of SEQ ID NO: 8.

In some embodiments, the antibody or antigen binding fragment thereof that binds GUCY2C comprises a VH1 which is at least 95% identical to the VH1 of SEQ ID NO: 7 and a VL1 which is identical to the VL1 of SEQ ID NO: 8.

In some embodiments, the antibody or antigen binding fragment thereof that binds GUCY2C comprises a VH1 which is identical to the VH1 of SEQ ID NO: 7 and a VL1 which is at least 95% identical to the VL1 of SEQ ID NO: 8.

In some embodiments, the antibody or antigen binding fragment thereof that binds GUCY2C comprises a VH1 which is at least 99% identical to the VH1 of SEQ ID NO: 7 and a VL1 which is at least 99% identical to the VL1 of SEQ ID NO: 8.

In some embodiments, the antibody or antigen binding fragment thereof that binds GUCY2C comprises a VH1 which is at least 95% identical to the VH1 of SEQ ID NO: 7 and a VL1 which is at least 99% identical to the VL1 of SEQ ID NO: 8.

In some embodiments, the antibody or antigen binding fragment thereof that binds GUCY2C comprises a VH1 which is at least 99% identical to the VH1 of SEQ ID NO: 7 and a VL1 which at least 95% is at identical to the VL1 of SEQ ID NO: 8.

In some embodiments, the antibody or antigen binding fragment thereof that binds GUCY2C comprises a VH1 which is at least 80% (e.g. at least 80%, at least 85%, at least 90%, at least 95%, at least 99% or 100%) identical to the VH1 of SEQ ID NO: 7 and a VL1 which is at least 80% (e.g. at least 80%, at least 85%, at least 90%, at least 95%, at least 99% or 100%) identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5 and 6, respectively.

In some embodiments, the antibody or antigen binding fragment thereof that binds GUCY2C comprises a VH1 which is at least 80% (e.g. at least 80%, least 85%, at least 90%, at least 95%, at least 99% or 100%) identical to the VH1 of SEQ ID NO: 7 and a VL1 which is identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a VH1 which is identical to the VH1 of SEQ ID NO: 7 and a VL1 which is at least 85% (e.g. at least 80%, least 85%, at least 90%, at least 95%, at least 99% or 100%) identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a VH1 which is at least 95% identical to the VH1 of SEQ ID NO: 7 and a VL1 which is at least 95% identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a VH1 which is at least 95% identical to the VH1 of SEQ ID NO: 7 and a VL1 which is identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a VH1 which is identical to the VH1 of SEQ ID NO: 7 and a VL1 which is at least 95% identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a VH1 which is at least 99% identical to the VH1 of SEQ ID NO: 7 and a VL1 which is at least 99% identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a VH1 which is at least 95% identical to the VH1 of SEQ ID NO: 7 and a VL1 which is at least 99% identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a VH1 which is at least 99% identical to the VH1 of SEQ ID NO: 7 and a VL1 which at least 95% is at identical to the VL1 of SEQ ID NO: 8, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is at least 80% (e.g. at least 80%, at least 85%, at least 90%, at least 95%, at least 99% or 100%) identical to the HC1 of SEQ ID NO: 9 and a LC1 which is at least 80% (e.g. at least 80%, at least 85%, at least 90%, at least 95%, at least 99% or 100%) identical to the LC1 of SEQ ID NO: 10.

In some embodiments, the antibody or antigen binding fragment thereof that binds GUCY2C comprises a HC1 which is at least 80% (e.g. at least 80%, least 85%, at least 90%, at least 95%, at least 99% or 100%) identical to the HC1 of SEQ ID NO: 9 and a LC1 which is identical to the LC1 of SEQ ID NO: 10.

In some embodiments, the antibody or antigen binding fragment thereof that binds GUCY2C comprises a HC1 which is identical to the HC1 of SEQ ID NO: 9 and a LC1 which is at least 85% (e.g. at least 80%, least 85%, at least 90%, at least 95%, at least 99% or 100%) identical to the LC1 of SEQ ID NO: 10.

In some embodiments, the antibody or antigen binding fragment thereof that binds GUCY2C comprises a HC1 which is at least 95% identical to the HC1 of SEQ ID NO: 9 and a LC1 which is at least 95% identical to the LC1 of SEQ ID NO: 10.

In some embodiments, the antibody or antigen binding fragment thereof that binds GUCY2C comprises a HC1 which is at least 95% identical to the HC1 of SEQ ID NO: 9 and a LC1 which is identical to the LC1 of SEQ ID NO: 10.

In some embodiments, the antibody or antigen binding fragment thereof that binds GUCY2C comprises a HC1 which is identical to the HC1 of SEQ ID NO: 9 and a LC1 which is at least 95% identical to the LC1 of SEQ ID NO: 10.

In some embodiments, the antibody or antigen binding fragment thereof that binds GUCY2C comprises a HC1 which is at least 99% identical to the HC1 of SEQ ID NO: 9 and a LC1 which is at least 99% identical to the LC1 of SEQ ID NO: 10.

In some embodiments, the antibody or antigen binding fragment thereof that binds GUCY2C comprises a HC1 which is at least 95% identical to the HC1 of SEQ ID NO: 9 and a LC1 which is at least 99% identical to the LC1 of SEQ ID NO: 10.

In some embodiments, the antibody or antigen binding fragment thereof that binds GUCY2C comprises a HC1 which is at least 99% identical to the HC1 of SEQ ID NO: 9 and a LC1 which at least 95% is at identical to the LC1 of SEQ ID NO: 10.

In some embodiments, the antibody or antigen binding fragment thereof that binds GUCY2C comprises a HC1 which is at least 80% (e.g. at least 80%, at least 85%, at least 90%, at least 95%, at least 99% or 100%) identical to the HC1 of SEQ ID NO: 9 and a LC1 which is at least 80% (e.g. at least 80%, at least 85%, at least 90%, at least 95%, at least 99% or 100%) identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is at least 80% (e.g. at least 80%, least 85%, at least 90%, at least 95%, at least 99% or 100%) identical to the HC1 of SEQ ID NO: 9 and a LC1 which is identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is identical to the HC1 of SEQ ID NO: 9 and a LC1 which is at least 85% (e.g. at least 80%, least 85%, at least 90%, at least 95%, at least 99% or 100%) identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is at least 95% identical to the HC1 of SEQ ID NO: 9 and a LC1 which is at least 95% identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is at least 95% identical to the HC1 of SEQ ID NO: 9 and a LC1 which is identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is identical to the HC1 of SEQ ID NO: 9 and a LC1 which is at least 95% identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is at least 99% identical to the HC1 of SEQ ID NO: 9 and a LC1 which is at least 99% identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is at least 95% identical to the HC1 of SEQ ID NO: 9 and a LC1 which is at least 99% identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

In some embodiments, the antibody or antigen binding fragment thereof that binds GUCY2C comprises a HC1 which is at least 99% identical to the HC1 of SEQ ID NO: 9 and a LC1 which at least 95% is at identical to the LC1 of SEQ ID NO: 10, wherein the antibody or antigen binding fragment comprises a HCDR1, a HCDR2, a HCDR3, a LCDR1, a LCDR2 and a LCDR3 of SEQ ID NOs 1, 2, 3, 4, 5, and 6, respectively.

### Variants of GUCY2CxCD3 Antibodies

The disclosure also provides variants of GUCY2CxCD3 bispecific antibodies.

In some embodiments, the GUCY2CxCD3 antibody or antigen binding fragment thereof comprises a first antigen binding domain that binds GUCY2C and a second binding site that binds CD3, wherein
the first antigen binding domain that binds GUCY2C comprises a heavy chain (HC1) which is at least 80% (e.g. at least 80%, at least 85%, at least 90%, at least 95%, or at least 99%) identical to SEQ ID NO: 9 and a light chain (LC1) which is at least 80% (e.g. at least 80%, at least 85%, at least 90%, at least 95%, or at least 99%) identical to SEQ ID NO: 10; and
the second binding domain that binds CD3 comprises a heavy chain (HC2) which is at least 80% (e.g. at least 80%, at least 85%, at least 90%, at least 95%, or at least 99%) identical to SEQ ID NO: 19.

In some embodiments, the GUCY2CxCD3 antibody or antigen binding fragment thereof comprises a first antigen binding domain that binds GUCY2C and a second binding site that binds CD3, wherein
the first antigen binding domain that binds GUCY2C comprises a VH1 which is at least 80% (e.g. at least 85%, at least 90%, at least 95%, or at least 99%) identical to SEQ ID NO: 7, a VL1 that is at least 80% (e.g. at least 85%, at least 90%, at least 95%, or at least 99%) identical to SEQ ID NO: 8; and
the second binding domain that binds CD3 comprises a spFv which is at least 80% identical to SEQ ID NO: 22.

In some embodiments, the GUCY2CxCD3 antibody or antigen binding fragment thereof comprises a first antigen binding domain that binds GUCY2C and a second binding site that binds CD3, wherein
the first antigen binding domain that binds GUCY2C comprises a VH1 which is at least 80% (e.g. at least 85%, at least 90%, at least 95%, or at least 99%) identical to SEQ ID NO: 7, a VL1 that is at least 80% (e.g. at least 85%, at least 90%, at least 95%, or at least 99%) identical to SEQ ID NO: 8; and the HCDR1, HCDR2, HCDR3, LCDR1, LCDR2 and LCDR3 of SEQ ID NOs: 1, 2, 3, 4, 5, and 6, respectively and
the second binding domain that binds CD3 comprises a spFv which is at least 80% identical to SEQ ID NO: 22.

### Human Antibodies

In some embodiments, the isolated antibody or isolated antigen binding fragment thereof that binds GUCY2C or the GUCY2CxCD3 antibody is a human antibody, a humanized antibody or a chimeric antibody.

“Chimeric antibodies” refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. (Morrison et al. (1984) Proc. Natl. Acad. Sci. USA 81, 6851; Neuberger et al. (1984) Nature 312, 604; and Takeda et al. (1984) Nature 314:452).

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both.

Typically, a “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

Transgenic animals, such as mice, rat or chicken carrying human immunoglobulin (Ig) loci in their genome may be used to generate antigen binding fragments that bind GUCY2C, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (www_regeneron_com), Harbour Antibodies (www_harbourantibodies_com), Open Monoclonal Technology, Inc. (OMT) (www_omtinc net), KyMab (www_kymab_com), Trianni (www.trianni_com) and Ablexis (www_ablexis_com) may be engaged to provide human antibodies directed against a selected antigen.

In some embodiments, the antibody or antigen binding fragment thereof that bind GUCY2C generated by immunizing non-human animals may be humanized. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen binding domains may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antigen binding domain.

### Polynucleotides and Vectors and Host Cells Comprising the Same

Further disclosed herein are isolated polynucleotides encoding the GUCY2C antibodies, and the GUCY2CxCD3 antibodies of the disclosure as well as the fragments and variants thereof.

In some embodiments, the disclosure provides isolated polynucleotide sequences encoding polypeptide sequences of SEQ ID NOs: 7 and 8.

In some embodiments, the disclosure provides isolated polynucleotide sequences encoding polypeptide sequences of SEQ ID NOs: 9 and 10.

In some embodiments, the disclosure provides isolated polynucleotide sequences encoding polypeptide sequences of SEQ ID NOs: 7, 8, and 22.

In some embodiments, the disclosure provides isolated polynucleotide sequences encoding polypeptide sequences of SEQ ID NOs: 9, 10 and 19.

In some embodiments, the disclosure provides an isolated polynucleotide of SEQ ID NO: 32, 33, 34, 35, 36, 37, 38 and 39.

The polynucleotides encoding the GUCY2C antibodies, GUCY2C-binding fragments thereof, the GUCY2CxCD3 antibodies and fragments thereof include polynucleotides with nucleic acid sequences that are substantially the same as the nucleic acid sequences of the polynucleotide of the disclosure. “Substantially the same” nucleic acid sequence is defined herein as a sequence with at least 80% identity to another nucleic acid sequence when the two sequences are aligned. Two nucleic acid sequences are substantially identical if the polypeptide encoded by the first nucleic acid is immunologically cross reactive with the polypeptide encoded by the second nucleic acid. Another indication that two nucleic acid sequences are substantially identical is that the two molecules hybridize to each other under stringent conditions.

Modified nucleotides may be used to generate the polynucleotides of the disclosure. Exemplary modified nucleotides are 5-fluorouracil, 5-bromouracil, 5-chlorouracil, 5-iodouracil, hypoxanthine, xanthine, 4-acetylcytosine, 5-(carboxyhydroxymethyl) uracil, carboxymethylaminomethyl-2-thiouridine, 5-carboxymethylaminomethyluracil, dihydrouracil, N6-substituted adenine, 7-methylguanine, 5-methylaminomethyluracil, 5-methoxyaminomethyl-2-thiouracil, beta-D-mannosylqueosine, 5″-methoxycarboxymethyluracil, 5-methoxyuracil, 2-methylthio-N6-isopentenyladenine, uracil-5-oxyacetic acid (v), wybutoxosine, pseudouracil, queuosine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, 2-thiocytosine, 5-methyl-2-thiouracil, 2-thiouracil, 4-thiouracil, 5-methyluracil, uracil-5-oxy acetic acid methylester, 3-(3-amino-3-N-2-carboxypropyl) uracil, and 2,6-diaminopurine.

### Vectors Comprising Polynucleotides Encoding the Anti-GUCY2C Antibodies

Vectors comprising DNA encoding the anti-GUCY2C antibodies, the GUCY2CxCD3 antibodies or the antigen binding fragments thereof of the disclosure are also provided. The disclosed vectors can be used, for example, to generate any of the above disclosed anti-GUCY2C antibodies, GUCY2CxCD3 antibodies or antigen binding fragments thereof. Polynucleotides encoding any of the anti-GUCY2C antibody, GUCY2CxCD3 antibody or antigen binding fragment thereof of the disclosure may be incorporated into vectors using standard molecular biology methods.

In some embodiments, the disclosure provides an expression vector comprising the polynucleotide of the invention. Such vectors may be plasmid vectors, viral vectors, vectors for baculovirus expression, transposon-based vectors or any other vector suitable for introduction of the synthetic polynucleotide of the invention into a given organism or genetic background by any means. The vector of the disclosure may be an expression vector for the efficient synthesis and expression of the anti-GUCY2C antibody polypeptide or GUCY2CxCD3 antibody polypeptide of the disclosure in prokaryotic and eukaryotic systems, including but not limited to yeast and mammalian cell culture. Also provided is a vector comprising, consisting of and/or consisting essentially of a nucleic acid encoding an anti-GUCY2C antibody, a GUCY2CxCD3 antibody or an antigen binding fragment thereof.

Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to certain embodiments. Such techniques are well known to those skilled in the art in view of the present disclosure.

Recombinant expression vectors within the scope of the description include synthetic, genomic, or cDNA-derived nucleic acid fragments that encode at least one recombinant protein which can be operably linked to suitable regulatory elements. Such regulatory elements can include a transcriptional promoter, sequences encoding suitable mRNA ribosomal binding sites, and sequences that control the termination of transcription and translation. Expression vectors, especially mammalian expression vectors, can also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host can also be incorporated.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza). Additional vectors include the pUC series (Fermentas Life Sciences, Glen Burnie, Md.), the pBluescript series (Stratagene, LaJolla, Calif), the pET series (Novagen, Madison, Wis.), the pGEX series (Pharmacia Biotech, Uppsala, Sweden), and the pEX series (Clontech, Palo Alto, Calif.). Bacteriophage vectors, such as kGT10, kGT11, kEMBL4, and kNM1149, kZapII (Stratagene) can be used. Exemplary plant expression vectors include pBI01, pBI01.2, pBIl21, pBI101.3, and pBIN19 (Clontech). Exemplary animal expression vectors include pEUK-Cl, pMAM, and pMAMneo (Clontech). The expression vector may be a viral vector, e.g., a retroviral vector, e.g., a gamma retroviral vector.

The vector of the disclosure may contain a promoter and an enhancer sequence. Polynucleotides encoding the GUCY2C binding proteins of the disclosure may be operably linked to control sequences in the expression vector(s) that ensure the expression of the GUCY2C binding proteins. Such regulatory elements may include a transcriptional promoter, sequences encoding suitable mRNA ribosomal binding sites, and sequences that control the termination of transcription and translation. Expression vectors may also include one or more non-transcribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

Vectors of the disclosure may also contain one or more Internal Ribosome Entry Site(s) (IRES). Inclusion of an IRES sequence into fusion vectors may be beneficial for enhancing expression of some proteins. In some embodiments, the vector system will include one or more polyadenylation sites (e.g., SV40), which may be upstream or downstream of any of the aforementioned nucleic acid sequences. Vector components may be contiguously linked or arranged in a manner that provides optimal spacing for expressing the gene products (i.e., by the introduction of “spacer” nucleotides between the ORFs) or positioned in another way. Regulatory elements, such as the IRES motif, may also be arranged to provide optimal spacing for expression.

Vectors of the disclosure may be circular or linear. They may be prepared to contain a replication system functional in a prokaryotic or eukaryotic host cell. Replication systems can be derived, e.g., from ColEl, SV40, 2μ plasmid, λ, bovine papilloma virus, and the like.

The recombinant expression vectors can be designed for either transient expression, for stable expression, or for both. Also, the recombinant expression vectors can be made for constitutive expression or for inducible expression.

The vectors may also comprise selection markers, which are well known in the art. Selection markers include positive and negative selection marker. Marker genes include biocide resistance, e.g., resistance to antibiotics, heavy metals, etc., complementation in an auxotrophic host to provide prototrophy, and the like. Exemplary marker genes include antibiotic resistance genes (e.g., neomycin resistance gene, a hygromycin resistance gene, a kanamycin resistance gene, a tetracycline resistance gene, a penicillin resistance gene, histidinol resistance gene, histidinol×resistance gene), glutamine synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

Numerous techniques are known in the art for the introduction of foreign genes into cells and can be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which can be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells can also be used to transform cells.

### Host Cells

The disclosure also provides for a host cell comprising any of the vectors of the disclosure. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-KlSV (Lonza Biologics, Walkersville, MD), CHO-K1 (ATCC CRL-61) or DG44.

Nucleic acids encoding any of the GUCY2C or CD3 binding proteins or fragments thereof can be used for transformation of a suitable mammalian host cell. Host cell transformation, culture, antibody expression and purification are done using well known methods. Also provided is a host cell comprising, consisting of and/or consisting essentially of a vector comprising, consisting of and/or consisting essentially of a nucleic acid encoding a anti-GUCY2C antibody, or a GUCY2CxCD3 antibody provided herein.

Cell lines may be selected based on high level of expression of the anti-GUCY2C antibody of interest and minimal contamination from host cell proteins. Mammalian cell lines available as host cells for expression are well known in the art and include, but are not limited to from Chinese Hamster Ovary (CHO) cells such as CHO-K1SV (Lonza Biologics, Walkersville, MD), CHO-K1 (ATCC CRL-61), or CHO DG44, and Baby Hamster Kidney (BHK) cells. These cell lines can be used to produce any of the anti-GUCY2C antibody, GUCY2CxCD3 antibody or antibody fragment thereof of the disclosure by culturing the cells under conditions suitable for expression of the antibody and purifying the antibody from the host cell or medium surrounding the host cell.

The disclosure also provides a method of producing the anti-GUCY2C antibody or the GUCY2CxCD3 antibody of the disclosure comprising culturing the host cell of the disclosure in conditions that the anti-GUCY2C antibody or GUCY2CxCD3 antibody is expressed, and recovering the anti-GUCY2C antibody or the GUCY2CxCD3 antibody produced by the host cell using well known methods in the art. A subject protein may be substantially pure, e.g., at least about 80% to 85% pure, at least about 85% to 90% pure, at least about 90% to 95% pure, or at least about 98% to 99%, or more, pure, e.g., free from contaminants such as cell debris, macromolecules, etc. other than the subject protein.

### Pharmaceutical Compositions

Further disclosed herein are pharmaceutical compositions comprising the disclosed GUCY2C antibodies and GUCY2C-binding fragments thereof, and the disclosed GUCY2CxCD3 antibodies and fragments thereof for the preparation of a medicament for treating a GUCY2C related condition, such as cancer.

For therapeutic use, the anti-GUCY2C antibody, GUCY2CxCD3 antibody and the antigen binding fragments thereof of the disclosure may be prepared as pharmaceutical compositions containing an effective amount of the antibody as an active ingredient in a pharmaceutically acceptable carrier.

“Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies in such pharmaceutical formulation may vary from less than about 0.5%, usually to at least about 1% to as much as 15 or 20% by weight and may be selected primarily based on required dose, fluid volumes, viscosities, etc., according to the mode of administration selected.

A pharmaceutically acceptable carrier can include a buffer, excipient, stabilizer, or preservative. The term “pharmaceutically acceptable,” as used herein with regard to pharmaceutical compositions, means approved by a regulatory agency of the Federal or a state government or listed in the U.S. Pharmacopeia or other generally recognized pharmacopeia for use in animals and/or in humans.

Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

Pharmaceutical compositions may comprise buffers such as acetic acid, citric acid, formic acid, succinic acid, phosphoric acid, carbonic acid, malic acid, aspartic acid, histidine, boric acid, Tris buffers, HEPPSO, HEPES, neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

Pharmaceutical compositions of the present disclosure can be formulated for a variety of means of parenteral or non-parenteral administration. In one embodiment, the compositions can be formulated for infusion or intravenous administration. Pharmaceutical compositions disclosed herein can be provided, for example, as sterile liquid preparations, e.g., isotonic aqueous solutions, emulsions, suspensions, dispersions, or viscous compositions, which may be buffered to a desirable pH. Formulations suitable for oral administration can include liquid solutions, capsules, sachets, tablets, lozenges, and troches, powders liquid suspensions in an appropriate liquid and emulsions.

### Method of Treatment and Uses

### Treatment Applications

Further disclosed is the use of any of the disclosed anti-GUCY2C antibodies, or any of the GUCY2CxCD3 antibodies and antigen binding fragments thereof for the treatment of cancer or for the killing of cancer cells overexpressing GUCY2C in a subject.

The disclosure provides methods comprising administering an effective amount of the antibodies or antibody fragments and any of pharmaceutical compositions disclosed herein to the subject, thereby killing the cancer cells. In one embodiment, the cancer cells are cells of the digestive system. In one embodiment, the cancers cells are cells of the gastrointestinal system. In one embodiment, the cancer cells are selected from colorectal cancer cells, gastric cancer cells, esophageal cancer cells and pancreatic cancer cells.

The disclosure also provides the use of any of the antibodies or antibody fragments and any of pharmaceutical compositions disclosed herein, for the treatment of GUCY2C related disease or disorder in a subject. In one embodiment, the GUCY2C related diseases or disorders are selected from cancers. In one embodiment, the cancer is selected from digestive system cancers. In one embodiment, the cancer is selected from gastrointestinal system cancers, such as colorectal cancer, gastric cancer, esophageal cancer, and pancreatic cancer.

In some embodiments, the disclosure provides methods of treating a GUCY2C related disease or disorder in a subject with any of the anti-GUCY2C antibodies or any of the GUCY2CxCD3 antibodies or antigen binding fragments thereof or any of the pharmaceutical composition of the disclosure.

In some embodiments, the disclosure provides methods of killing cancer cells overexpressing GUCY2C in a subject with any of the anti-GUCY2C antibodies or any of the GUCY2CxCD3 antibodies or antigen binding fragments thereof or any of the pharmaceutical composition of the disclosure.

In some embodiments, the disclosure provides methods of treating a GUCY2C related cancer in a subject with any of the anti-GUCY2C antibodies or any of the GUCY2CxCD3 antibodies or antigen binding fragments thereof or any of the pharmaceutical composition of the disclosure.

“Treat,” “treating,” or “treatment” of a disease or disorder such as cancer refers to accomplishing one or more of the following: reducing the severity and/or duration of the disorder, delaying the progression of the disorder, slowing the progression of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder. As used herein, the terms “delaying the progression of” or “slowing the progression of” shall include (a) delaying or slowing the development of one or more symptoms or complications of the disease, condition or disorder; (b) delaying or slowing the development of one or more new/additional symptoms or complications of the disease, condition or disorder; and/or (c) delaying or slowing the progression of the disease, condition or disorder to a later stage or more serious form of said disease, condition or disorder.

“Subject” includes any human or non-human animal. “Non-human animal” includes all vertebrates, e.g., mammals and non-mammals. The term “mammal” as used herein, encompasses any mammal. Examples of mammals include, but are not limited to, non-human primates, cows, horses, sheep, pigs, cats, dogs, mice, rats, rabbits, guinea pigs, monkeys, humans, etc. The terms “subject” and “patient” can be used interchangeably herein. In some embodiments, the subject or patient is human.

A “effective amount” refers to an amount effective, at doses and for periods of time necessary, to achieve a desired therapeutic result. Such concentrations can be routinely determined by those of skilled in the art. An effective amount may vary depending on factors such as the disease state, age, sex, and weight of the individual, the physical condition of the patient, the duration of the treatment, the nature of concurrent therapy (if any), the specific formulations employed, the structure of the antibody or antibody fragment or of its variants and the ability of a therapeutic or a combination of therapeutics to elicit a desired response in the individual. An effective amount of the administered antibody or antibody fragment may depend on the type and severity of the cancer being treated, and the route of administration of the antibody polypeptide or the pharmaceutical composition of the antibody.

“Cancer” is meant to include all types of cancerous growths or oncogenic processes, metastatic tissues or malignantly transformed cells, tissues, or organs, irrespective of histopathology type or stage of invasiveness. Examples of cancers include solid tumors, hematological malignancies, soft tissue tumors, and metastatic lesions. Exemplary solid tumors include malignancies, e.g., sarcomas, and carcinomas (including adenocarcinomas and squamous cell carcinomas) of the various organ systems, such as those affecting prostate, liver, lung, breast, lymphoid, gastrointestinal (e.g., colon), genitourinary tract (e.g., renal, urothelial cells), prostate and pharynx. Adenocarcinomas include malignancies such as most colon cancers, a rectal cancer, a renal-cell carcinoma, a liver cancer, a non-small cell carcinoma of the lung, a cancer of the small intestine and a cancer of the esophagus. Squamous cell carcinomas include malignancies, e.g., in the lung, esophagus, skin, head and neck region, oral cavity, anus, and cervix.

The cancer can be a hyperproliferative condition or disorder, a solid tumor, a neovasculature, a soft tissue tumor, or a metastatic lesion.

In some embodiments, the cancer is early-stage cancer, non-metastatic cancer, primary cancer, advanced cancer, locally advanced cancer, metastatic cancer, cancer in remission, recurrent cancer, cancer in an adjuvant setting, cancer in a neoadjuvant setting, or cancer substantially refractory to a therapy.

The term “GUCY2C positive cancer”, “GUCY2C expressing cancer” or similar terms means a cancer involving cancer cells expressing a GUCY2C.

In some embodiment the GUCY2C positive cancer is a cancer of the digestive system.

In some embodiment the GUCY2C positive cancer includes but is not limited to colorectal cancer, gastric cancer, esophageal cancer, and pancreatic cancer.

In some embodiments, the cancer is a colorectal cancer.

In some embodiments, the cancer is advanced or metastatic colorectal cancer.

In some embodiments, the cancer is unresectable metastatic colorectal cancer.

In some embodiments, the cancer is a gastric cancer.

In some embodiments, the cancer is a esophageal cancer.

In some embodiments, the cancer is a pancreatic cancer.

In some embodiments, the disclosure provides methods of killing cancer cells overexpressing GUCY2C and/or method of treating a GUCY2C expressing cancer in a subject comprising administering to the subject an antibody, or an antigen binding fragment thereof that specifically binds to GUCY2C wherein the antibody, or an antigen binding domain that binds GUCY2C comprises
(i) the HCDRs of a VH1 comprising an amino acid sequence of SEQ ID NO: 7; and the LCDRs of a VL1 comprising an amino acid sequence of SEQ ID NO: 8;
(ii) a VH1 comprising a HCDR1 having an amino acid sequence of SEQ ID NO: 1 a HCDR2 having an amino acid sequence of SEQ ID NO: 2 and a HCDR3 having an amino acid sequence of SEQ ID NO: 3; and a VL1 comprising a LCDR1 having an amino acid sequence of SEQ ID NO: 4, a LCDR2 having an amino acid sequence of and SEQ ID NO: 5 and a LCDR3 having an amino acid sequence of SEQ ID NO: 6;
(iii) a VH1 having at least 80%, at least 85%, at least 90%, at least 95%, or at least 98% sequence identity to the amino acid sequence of SEQ ID NO: 7, and/or a VL1 having at least 80%, at least 85%, at least 90%, at least 95%, or at least 98% sequence identity to the amino acid sequence of SEQ ID NO: 8;
(iv) a HC1 having at least 80%, at least 85%, at least 90%, at least 95%, or at least 98% sequence identity to an amino acid sequence of SEQ ID NO: 9 and a LC1 having at least 80%, at least 85%, at least 90%, at least 95%, or at least 98% sequence identity to an amino acid sequence of SEQ ID NO: 10; or
(iv) a HC1 having of SEQ ID NO: 9 and LC1 of SEQ ID NO: 10 In some embodiments, the disclosure provides methods of killing cancer cells overexpressing GUCY2C and/or method of treating a GUCY2C expressing cancer, comprising administering to the subject an GUCY2CxCD3 antibody, or antigen binding fragment thereof that comprises a first binding domain that binds GUCY2C and a second binding domain that binds CD3, wherein the first binding domain that binds GUCY2C comprises
(i) the HCDR1, HCDR2, HCDR3 of a VH1 comprising an amino acid sequence of SEQ ID NO: 7; and the LCDR1, LCDR2 and LCDR3 of a VL1 comprising an amino acid sequence of SEQ ID NO: 8;
(ii) a VH1 comprising a HCDR1, a HCDR2, and a HCDR3 having an amino acid sequence of SEQ ID NOs: 1, 2, and 3, respectively, and a VL1 comprising a LCDR1, LCDR2, and LCDR3 having an amino acid sequence of SEQ ID NOs: 3, 4, and 5 respectively;
(iii) a VH1 having an amino acid sequence of SEQ ID NO: 7 and a VL1 having an amino acid sequence of SEQ ID NO: 8; and/or
(iv) a HC1 having an amino acid sequence of SEQ ID NO: 9 and a LC1 having an amino acid sequence of SEQ ID NO: 10; and the second binding domain that binds CD3 comprises
(i) a spFv having an amino acid sequence of SEQ ID NO: 22; or
(ii) a HC2 having an amino acid sequence of SEQ ID NO: 19.

In some embodiments, the disclosure provides a GUCY2CxCD3 antibody, an antigen binding fragment thereof, or a pharmaceutical composition for use in combination therapy. In some embodiments, the GUCY2CxCD3 bispecific antibody of the disclosure may be used in combination with a supplemental therapy. In some embodiments, the supplemental therapy is surgery. In some embodiments, the supplemental therapy is radiation. In some embodiments, the supplemental therapy is chemotherapy. In some embodiments, the GUCY2CxCD3 bispecific antibody of the disclosure is administered in combination with one or more pharmaceutically acceptable ingredient(s), active principle(s) or composition(s).

### Diagnostic Applications

The GUCY2C antibodies or antibody fragments disclosed herein may be used in a variety of assays to detect GUCY2C in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

Any of the GUCY2C antibodies or antibody fragments disclosed herein may be used for detecting and quantifying GUCY2C protein levels in a biological sample using a conventional method, for example, any immunohistological method known to those of skill in the art. Other antibody-based methods useful for detecting GUCY2C protein expression include immunoassays, such as the enzyme linked immunosorbent assay (ELISA), immunoprecipitation, or Western blotting. Suitable assays are described in more detail elsewhere herein. The term “biological sample” means any biological sample obtained from an individual, cell line, tissue culture, or other source of cells potentially expressing GUCY2C. Methods for obtaining tissue biopsies and body fluids from mammals are well known in the art.

### Kit Comprising the Antibody, Host Cell or Vector of the Disclosure

The present disclosure also provides kits comprising any of the anti-GUCY2C antibodies, any of the GUCY2CxCD3 antibodies or antigen binding fragments thereof disclosed herein. Such kits can be used for any of the applications of such antibodies as disclosed herein, for example, for use in treating or alleviating a target disease, such as a cancer as disclosed herein, or for detecting the presence or measuring the amount of GUCY2C protein or GUCY2C positive cells in a biological sample. Such kits can include one or more containers comprising an anti-GUCY2C antibody or an GUCY2CxCD3 antibody described herein.

In some embodiments, the kit can comprise instructions for use in accordance with any of the methods described herein. The included instructions can comprise a description of administration of the anti-GUCY2C antibody or the GUCY2CxCD3 antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of the anti-GUCYC antibody or the GUCY2CxCD3 antibody generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

Kits may optionally provide additional components such as buffers and interpretive information. Normally, the kit comprises a container and a label or package insert(s) on or associated with the container. In some embodiments, the invention provides articles of manufacture comprising contents of the kits described above.

In some embodiments, the disclosure provides a kit comprising an antibody or antigen binding fragment thereof that binds GUCY2C.

In some embodiments, the disclosure provides a kit comprising a GUCY2CxCD3 antibody or antigen binding fragment thereof that binds GUCY2C and CD3.

The kit may be used for therapeutic uses or for as diagnostic use.

The kit may be used to detect the presence of GUCY2C in a sample.

In some embodiments, the kit comprises the anti-GUCY2C antibody or antigen binding fragment of the disclosure and reagents for detecting the GUCY2C binding protein. In some embodiments, the kit comprises the GUCY2CxCD3 antibody or antigen binding fragment thereof of the disclosure and reagents for detecting the GUCY2C binding protein. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the disclosed GUCY2C antibody, antigen binding fragment thereof or the disclosed GUCY2CxCD3 antibody or antigen binding fragment thereof or a pharmaceutical composition in a container and instructions for use of the kit.

In some embodiments, the kit comprises an antibody, or antigen binding fragment thereof that binds GUCY2C, wherein the antibody or antigen binding fragment comprises a heavy chain variable region (VH1) and a light chain variable region (VL1) of SEQ ID NO: 7 and 8, respectively.

In some embodiments, the kit comprises an anti-GUCY2C antibody or antigen binding fragment thereof comprising a heavy chain variable region (VH1) comprising a HCDR1 having an amino acid sequence of SEQ ID NO: 1, a HCDR2 having an amino acid sequence of and SEQ ID NO: 2 and a HCDR3 having an amino acid sequence of SEQ ID NO: 3; a light chain variable region (VL1) comprising a LCDR1 having an amino acid sequence of SEQ ID NO: 4, a LCDR2 having an amino acid sequence of and SEQ ID NO: 5 and a LCDR3 having an amino acid sequence of SEQ ID NO: 6; and/or
a heavy chain variable region (VH1) having at least 80%, at least 85%, at least 90%, at least 95%, or at least 98% sequence identity to the amino acid sequence of SEQ ID NO: 7, a light chain variable region (VL1) having at least 80%, at least 85%, at least 90%, at least 95%, or at least 98% sequence identity to the amino acid sequence of SEQ ID NO: 8.

In some embodiments, the kit comprises a GUCY2CxCD3 antibody or antigen binding fragment thereof comprising a first binding domain that binds GUCY2C, wherein the first binding domain that binds GUCY2C comprises a VH1 and a VL1 comprising the amino acid sequence of SEQ ID NO: 7 and 8, respectively.

In some embodiments, the kit comprises a GUCY2CxCD3 antibody or antigen binding fragment thereof comprising a first binding domain that binds GUCY2C, wherein the first binding domain that binds GUCY2C comprises a HC1 and a LCT comprising the amino acid sequence of SEQ ID NO: 9 and 10, respectively.

In some embodiments, the kit comprises a GUCY2CxCD3 antibody or antigen binding fragment thereof comprising a heavy chain complementarity determining region 1 (HCDR1), a HCDR2, and a HCDR3, and a light chain complementarity determining region 1 (LCDR1), a LCDR2, and LCDR3 of SEQ ID NOs: 1, 2, 3, 4, 5, and 6, respectively.

In some embodiments, the kit comprises a GUCY2CxCD3 antibody or antigen binding fragment thereof comprising a first binding domain that binds GUCY2C and a second binding domain that binds CD3, wherein the first binding domain that binds GUCY2C comprises a VH1 and a VL1 comprising the amino acid sequence of SEQ ID NO: 7 and 8, respectively; and wherein the second binding domain that binds CD3 comprises a spFv of SEQ ID NO: 22.

In some embodiments, the kit comprises a GUCY2CxCD3 antibody or antigen binding fragment thereof comprising a first binding domain that binds GUCY2C and a second binding domain that binds CD3, wherein the first binding domain that binds GUCY2C comprises a heavy chain (HC1) and a light chain (LC1) comprising the amino acid sequence of SEQ ID NO: 9 and 10, respectively; and wherein the second binding domain that binds CD3 comprises a heavy chain (HC2) of SEQ ID NO: 19.

### Embodiments

The following list of embodiments is intended to complement, rather than displace or supersede, the previous descriptions.

Embodiment 1. An isolated antibody or antigen binding fragment thereof comprising a first binding domain, wherein the first binding domain binds to guanylyl cyclase C (GUCY2C), and wherein the first binding domain comprises a first heavy chain complementarity determining region (HCDR) 1, a first HCDR2, and a first HCDR3 of a first heavy chain variable region (VH1) of SEQ ID NO: 7 and a first light chain complementarity determining region (LCDR) 1, a first LCDR2, and a first LCDR3 of a first light chain variable region (VL1) of SEQ ID NO: 8, wherein the HCDR1, HCDR2, HCDR3, LCDR1, LCDR2, and LCDR3 amino acid sequences preferably are according to the Kabat numbering system, the Chothia numbering system, the AbM numbering system, the Contact numbering system or the IMGT numbering system or a combination thereof, preferably according to the Kabat numbering system, the Chothia numbering system, the AbM numbering system, the Contact numbering system or the IMGT numbering system.

Embodiment 2. The isolated antibody or antigen binding fragment there of embodiment 1, which specifically binds to GUCY2C.

Embodiment 3. The isolated antibody or antigen binding fragment thereof of embodiment 1 or 2, wherein the first binding domain comprises the first HCDR1, the first HCDR2, the first HCDR3, the first LCDR1, the first LCDR2, and the first LCDR3 of
a. SEQ ID NOs: 1, 2, 3, 4, 5, 6, respectively;
b. SEQ ID NO: 23, 24, 3, 4, 5, and 6, respectively;
c. SEQ ID NO: 25, 26, 3, 4, 5, and 6, respectively;
d. SEQ ID NO: 27, 28, 79, 29, 30 and 32, respectively; or e. SEQ ID NO: 80, 81, 82, 83, the amino acid sequence GAS, and SEQ ID NO: 6, respectively.

Embodiment 4. The isolated antibody or antigen binding fragment thereof of any one of embodiment 1-3, wherein the VH1 comprises an amino acid sequence which is at least 90%, or at least 95%, or at least 98%, at least 99%, or 100% identical to SEQ ID NO: 7.

Embodiment 5. The isolated antibody or antigen binding fragment thereof of any one of embodiments 1-4, wherein the VL1 comprises an amino acid sequence which is at least 90%, or at least 95%, or at least 98%, or at least 99% or 100% identical to SEQ ID NO: 8.

Embodiment 6. The isolated antibody or antigen binding fragment thereof of any one of embodiments 1-5, wherein the VH1 comprises an amino acid sequence of SEQ ID NO: 7 and the VL1 comprises an amino acid sequence of SEQ ID NO: 8.

Embodiment 7. The isolated antibody or antigen binding fragment thereof of any one of embodiments 1-6, wherein the first binding domain is or comprises an antibody fragment selected from the group consisting of Fv fragments, single chain fragments (scFv), (scFv)2, single chain disulfide bond stabilized fragments, stapled scFv fragment (spFv), Fab, F(ab′)2, dAb, VHH, and single chain antibodies.

Embodiment 8. The isolated antibody or antigen binding fragment thereof of any one of embodiments 1-7, wherein the first binding domain is or comprises a Fab.

Embodiment 9. The isolated antibody or antigen binding fragment thereof of any one of embodiments 1-8, which is monospecific.

Embodiment 10. The isolated antibody or antigen binding fragment thereof of any one of embodiments 1-8 which is multispecific.

Embodiment 11. The isolated antibody or antigen binding fragment thereof of embodiment 10, which is bispecific.

Embodiment 12. The isolated antibody or antigen binding fragment thereof of embodiment 10, which is trispecific.

Embodiment 13. The isolated antibody or antigen binding fragment thereof of any one of embodiments 10-12, which further comprises a second binding domain that binds an antigen on a lymphocyte.

Embodiment 14. The isolated antibody or antigen binding fragment thereof of embodiment 13, wherein the lymphocyte is a T cell.

Embodiment 15. The isolated antibody or antigen binding fragment thereof of embodiment 14, wherein the T cell is a CD8+ T cell.

Embodiment 16. The isolated antibody or antigen binding fragment thereof of embodiment 13, wherein the lymphocyte is a natural killer (NK) cell.

Embodiment 17. The isolated antibody or antigen binding fragment thereof of embodiment 13, wherein the antigen on the lymphocyte is CD3, CD8, BTNL3, CD186, BTNL8, PD-1, or CD195.

Embodiment 18. The isolated antibody or antigen binding fragment thereof of embodiment 13, wherein the antigen on the lymphocyte is K12L4, NKG2E, NKG2D, NKG2F, BTNL3, CD186, BTNL8, PD-1, or NKG2C.

Embodiment 19. The isolated antibody or antigen bind fragment thereof of any one of embodiments 13-15 and 17, wherein the second binding domain binds to CD3 epsilon (CD3c).

Embodiment 20. The isolated antibody or antigen binding fragment thereof of any one of embodiments 13-15, 17 and 19, which binds or specifically binds to GUCY2C and CD3c.

Embodiment 21. The isolated antibody or antigen binding fragment thereof of embodiment 19 or 20, which binds to CD3E with an EC50 of about 50-200 nM, about 60-180 nM, about 70-170 nM, or about 75-160 nM.

Embodiment 22. The isolated antibody or antigen binding fragment thereof of any one of embodiments 19-21, wherein the second binding domain comprises a second HCDR1, a second HCDR2, and a second HCDR3 of a second VH (VH2) of SEQ ID NO: 17 and a second LCDR1, a second LCDR2, and a second LCDR3 of a second VL (VL2) of SEQ ID NO: 18.

Embodiment 23. The isolated antibody or antigen binding fragment thereof of any one of embodiments 19-22, wherein the second binding domain comprises the second HCDR1, the second HCDR2, the second HCDR3, the second LCDR1, the second LCDR2, and the second LCDR3 of SEQ ID NOs: 11, 12, 13, 14, 15, 16, respectively.

Embodiment 24. The isolated antibody or antigen binding fragment thereof of embodiment 22 or 23, wherein the VH2 comprises an amino acid sequence which is at least 90%, or at least 95%, or at least 98%, or at least 99%, or 100% identical to SEQ ID NO: 17.

Embodiment 25. The isolated antibody or antigen binding fragment thereof of any one of embodiments 22-24, wherein the VL2 comprises an amino acid sequence which is at least 90%, or at least 95%, or at least 98%, or at least 99% or 100% identical to SEQ ID NO: 18.

Embodiment 26. The isolated antibody or antigen binding fragment thereof of any one of embodiments 13-25 wherein the second binding domain is an antibody fragment selected from the group consisting of Fv fragments, single chain fragments (scFv), (scFv)2, single chain disulfide bond stabilized fragments, stapled scFv fragment (spFv), Fab, F(ab′)2, dAb, VHH, and single chain antibodies.

Embodiment 27. The isolated antibody or antigen binding fragment thereof of embodiment 26, wherein the second binding domain is a scFv.

Embodiment 28. The isolated antibody or antigen binding fragment thereof of embodiment 27, wherein the second binding domain is a stapled scFv (spFv).

Embodiment 29. The isolated antibody or antigen binding fragment thereof of embodiment 28, wherein the spFv comprises the VH2 of SEQ ID NO: 17 and the VL2 of SEQ ID NO: 18.

Embodiment 30. The isolated antibody or antigen binding fragment thereof of embodiment 28 or 29, wherein the spFv is in an “VH2-linker-VL2” orientation or in a “VL2-linker-VH2” orientation.

Embodiment 31. The isolated antibody or antigen binding fragment thereof of embodiment 30, wherein the linker is selected from the group consisting of SEQ ID NOs: 40-44.

Embodiment 32. The isolated antibody or antigen binding fragment thereof of embodiment 31, wherein the spFv is in the orientation of “VL2-linker-VH2” and the linker has an amino acid sequence of SEQ ID NO: 40.

Embodiment 33. The isolated antibody or antigen binding fragment thereof of any one of embodiments 28-32, wherein the spFv comprises the sequence of SEQ ID NO: 22.

Embodiment 34. The isolated antibody or antigen binding fragment thereof of anyone of embodiments 1-33, which is of an IgG1, an IgG2, an IgG3, or an IgG4 isotype.

Embodiment 35. The isolated antibody or antigen binding fragment thereof of embodiment 34, which is of an IgG1 isotype.

Embodiment 36. The isolated antibody or antigen binding fragment thereof of any one of embodiments 1-35, wherein the first binding domain further comprises an IgG constant region or a fragment of the IgG constant region.

Embodiment 37. The isolated antibody or antigen binding fragment thereof any one of embodiments 10-36, which comprises a second binding domain, and wherein the second binding domain further comprises an IgG constant region or a fragment of the IgG constant region.

Embodiment 38. The isolated antibody or antigen binding fragment thereof of embodiment 36 or 37, wherein the IgG constant region, or the fragment of the IgG constant region, comprises at least one mutation that results in reduced binding of the antibody or antigen binding fragment thereof to a Fcγ receptor (FcγR).

Embodiment 39. The isolated antibody or antigen binding fragment thereof of embodiment 38, wherein the at least one mutation that results in reduced binding of the antibody or antigen binding fragment thereof to the FcγR is selected from the group consisting of F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S, and S228P/F234A/L235A/G236-deleted/G237A/P238S, wherein residue numbering is according to the EU index.

Embodiment 40. The isolated antibody or antigen binding fragment thereof of embodiment 38 or 39, wherein the at least one mutation that results in reduced binding of the antibody or antigen binding fragment thereof to the FcγR are L234A/L235A/D265S.

Embodiment 41. The isolated antibody or antigen binding fragment thereof of embodiment 36 or 37, wherein the IgG constant region or the fragment of the IgG constant region comprises at least one mutation that results in enhanced binding of the antibody or antigen binding fragment thereof to FcγR.

Embodiment 42. The isolated antibody or antigen binding fragment thereof of embodiment 41, wherein the at least one mutation that results in enhanced binding of the antibody or antigen binding fragment thereof to the FcγR is selected from the group consisting of S239D/1332E, S298A/E333A/K334A, F243L/R292P/Y300L, F243L/R292P/Y300L/P396L, F243L/R292P/Y300L/V305I/P396L, and G236A/S239D/I332E, wherein residue numbering is according to the EU index.

Embodiment 43. The isolated antibody or antigen binding fragment thereof of any one of embodiments 38-42, wherein the FcγR is FcγRI, FcγRIIA, FcγRIIB, FcγRIII, or any combination thereof.

Embodiment 44. The isolated antibody or antigen binding fragment thereof of any one of embodiments 36-44, wherein the IgG constant region or the fragment of the IgG constant region, further comprises at least one mutation that modulates a half-life of the antibody or antigen binding fragment thereof.

Embodiment 45. The isolated antibody or antigen binding fragment thereof of embodiment 44, wherein the at least one mutation that modulates the half-life of the antibody or antigen binding fragment thereof is selected from the group consisting of H435A, P257I/N434H, D376V/N434H, M252Y/S254T/T256E/H433K/N434F, M252Y/S254T/T256E, T308P/N434A, and H435R, wherein residue numbering is according to the EU index.

Embodiment 46. The isolated antibody or antigen binding fragment thereof of any one of embodiments 19-45, wherein the first binding domain is or comprises a first heavy chain (HC1) which comprises a Fab comprising a VH1 fused to a CH1, CH2, and CH3 of a human IgG1 heavy chain and a first light chain (LC1) comprising a VL1 fused to a CL of human kappa light chain, and wherein the second binding domain comprises a second heavy chain (HC2) comprising a spFv in a “VL2-linker-VH2” orientation fused to a CH2 and CH3 of a human IgG1 heavy chain.

Embodiment 47. The isolated antibody or antigen binding fragment thereof of embodiment 46, wherein the HC1 comprises the T366S, L368A, and Y407V mutations and the HC2 comprises the T366W mutation (numbering is according to EU index); or wherein the HC1 comprises the T366W mutation and the HC2 comprises the T366S, L368A, and Y407V mutations (numbering is according to EU index).

Embodiment 48. The isolated antibody or antigen binding fragment thereof of any one of embodiments 46-47, wherein the HC1 comprises an amino acid sequence at least 90%, at least 95%, at least 98%, at least 99% or 100% identical to SEQ ID NO: 9.

Embodiment 49. The isolated antibody or antigen binding fragment thereof of any one of embodiments 46-47, wherein the LC1 comprises an amino acid sequence at least 90%, at least 95%, at least 98%, at least 99% or 100% identical to SEQ ID NO: 10.

Embodiment 50. The isolated antibody or antigen binding fragment thereof any one of embodiments 46-49, wherein the HC1 comprises an amino acid sequence of SEQ ID NO: 9 and the LC1 comprises an amino acid sequence of SEQ ID NO: 10.

Embodiment 51. The isolated antibody or antigen binding fragment thereof of any one of embodiments 46-50, wherein the HC2 comprises an amino acid sequence at least 90%, at least 95%, at least 98%, at least 99% or 100% identical to SEQ ID NO: 19.

Embodiment 52. The isolated antibody or antigen binding fragment thereof of embodiments 46-51, wherein the HC2 comprises an amino acid of SEQ ID NO: 19.

Embodiment 53. The isolated antibody or antigen binding fragment thereof of any one of embodiments 46-52, which is bispecific and wherein the HC1 comprises an amino acid sequence of SEQ ID NO: 9, the LC1 comprises an amino acid sequence of SEQ ID NO: 10, and the HC2 comprises an amino acid sequence of SEQ ID NO: 19.

Embodiment 54. A pharmaceutical composition comprising the isolated antibody or antigen binding fragment thereof of any one of embodiments 1-53.

Embodiment 55. A polynucleotide encoding the isolated antibody or antigen binding fragment thereof of any one of embodiments 1-53.

Embodiment 56. A vector, preferably an expression vector, comprising the polynucleotide of embodiment 55.

Embodiment 57. A host cell, preferably a host CHO cell, expressing the isolated antibody or antigen binding fragment thereof of any one of embodiments 1-53.

Embodiment 58. A host cell, a preferably host CHO cells, comprising the vector, preferably the expression vector, of embodiment 56.

Embodiment 59. An in vitro or in vivo method of killing cancer cells overexpressing GUCY2C, comprising contacting the isolated antibody or antigen binding fragment thereof of any one of embodiments 1-53 or the pharmaceutical composition of embodiment 54 with the cancer cells, thereby killing the cancer cells.

Embodiment 60. A method of killing cancer cells overexpressing GUCY2C in a subject, comprising administering the isolated antibody or antigen binding fragment thereof of any one of embodiments 1-53 or the pharmaceutical composition of embodiment 54 to the subject, thereby killing the cancer cells.

Embodiment 61. The method of embodiment 60 wherein the subject is a human subject.

Embodiment 62. The method of any one of embodiment 60 or 61, wherein the cancer cells are cells of the digestive system, preferably of the gastrointestinal system.

Embodiment 63. The method of any one of embodiments 60-62, wherein the cancer cells are selected from colorectal cancer cells, gastric cancer cells, esophageal cancer cells and pancreatic cancer cells.

Embodiment 64. The method of any one of embodiments 60-63, wherein the cancer cells are colorectal cancer cells.

Embodiment 65. The method of any one of embodiments 60-63, wherein the cancer cells are advanced or metastatic colorectal cancer cells.

Embodiment 66. The method of embodiment 65, wherein the cancer cells are unresectable metastatic colorectal cancer cells.

Embodiment 67. The method of any one of embodiments 60-63, wherein the cancer cells are gastric cancer cells.

Embodiment 68. The method of any one of embodiments 60-63, wherein the cancer cells are esophageal cancer cells.

Embodiment 69. The method of any one of embodiments 60-63, wherein the cancer cells are pancreatic cancer cells.

Embodiment 70. The isolated antibody or antigen binding fragment thereof of any one of embodiments 1-53 or the pharmaceutical composition of embodiment 54, for use in the treatment of cancer.

Embodiment 71. The isolated antibody or antigen binding fragment thereof or the pharmaceutical composition for the use of embodiment 70, wherein the cancer is or comprises a cancer of the digestive system, preferably of the gastrointestinal system.

Embodiment 72. The isolated antibody or antigen binding fragment thereof or the pharmaceutical composition for the use of embodiment 70 or 71, wherein the cancer is or comprises a cancer selected from colorectal cancer, gastric cancer, esophageal cancer and pancreatic cancer.

Embodiment 73. The isolated antibody or antigen binding fragment thereof or the pharmaceutical composition for the use of any one of embodiments 70-72, wherein the cancer is or comprises colorectal cancer.

Embodiment 74. The isolated antibody or antigen binding fragment thereof or the pharmaceutical composition for the use of any one of embodiments 70-72, wherein the cancer is or comprises advanced or metastatic colorectal cancer.

Embodiment 75. The isolated antibody or antigen binding fragment thereof or the pharmaceutical composition for the use of embodiment 74, wherein the cancer is or comprises unresectable metastatic colorectal cancer.

Embodiment 76. The isolated antibody or antigen binding fragment thereof or the pharmaceutical composition for the use of any one of embodiments 70-72, wherein the cancer is or comprises gastric cancer.

Embodiment 77. The isolated antibody or antigen binding fragment thereof or the pharmaceutical composition for the use of any one of embodiments 70-72, wherein the cancer is or comprises esophageal cancer.

Embodiment 78. The isolated antibody or antigen binding fragment thereof or the pharmaceutical composition for the use of any one of embodiments 70-72, wherein the cancer is or comprises pancreatic cancer.

Embodiment 79. The isolated antibody or antigen binding fragment thereof of any one of embodiments 1-53 or the pharmaceutical composition of embodiment 54, for use as a medicament.

Embodiment 80. The isolated antibody or antigen binding fragment thereof of any one of embodiments 1-53 or the pharmaceutical composition of embodiment 54, for use as a medicament for cancer.

Embodiment 81. The isolated antibody or antigen binding fragment thereof of any one of embodiments 1-53 or the pharmaceutical composition of embodiment 54, for use as a medicament for cancer of the digestive system, preferably of the gastrointestinal system.

Embodiment 82. The isolated antibody or antigen binding fragment thereof or the pharmaceutical composition for the use of embodiment 80 or 81, wherein the cancer is or comprises a cancer selected from colorectal cancer, gastric cancer, esophageal cancer and pancreatic cancer.

Embodiment 83. The isolated antibody or antigen binding fragment thereof or the pharmaceutical composition for the use of any one of embodiments 80-81, wherein the cancer is or comprises colorectal cancer.

Embodiment 84. The isolated antibody or antigen binding fragment thereof or the pharmaceutical composition for the use of any one of embodiments 80-82, wherein the cancer is or comprises advanced or metastatic colorectal cancer.

Embodiment 85. The isolated antibody or antigen binding fragment thereof or the pharmaceutical composition for the use of embodiment 84, wherein the cancer is or comprises unresectable metastatic colorectal cancer.

Embodiment 86. The isolated antibody or antigen binding fragment thereof or the pharmaceutical composition for the use of any one of embodiments 80-81, wherein the cancer is or comprises gastric cancer.

Embodiment 87. The isolated antibody or antigen binding fragment thereof or the pharmaceutical composition for the use of any one of embodiments 80-81, wherein the cancer is or comprises esophageal cancer.

Embodiment 88. The isolated antibody or antigen binding fragment thereof or the pharmaceutical composition for the use of any one of embodiments 80-81, wherein the cancer is or comprises pancreatic cancer.

Embodiment 89. The isolated antibody or antigen binding fragment thereof of any one of embodiments 1-53 or the pharmaceutical composition of embodiment 54, for use in the manufacture of a medicament.

Embodiment 90. The isolated antibody or antigen binding fragment thereof of any one of embodiments 1-53 or the pharmaceutical composition of embodiment 54, for use in the manufacture of a medicament for cancer.

Embodiment 91. The isolated antibody or antigen binding fragment thereof of any one of embodiments 1-53 or the pharmaceutical composition of embodiment 54, for use in the manufacture of a medicament for cancer of the digestive system, preferably of the gastrointestinal system.

Embodiment 92. The isolated antibody or antigen binding fragment thereof, or the pharmaceutical composition, for the use of embodiment 90 or 91, wherein the cancer is or comprises a cancer selected from colorectal cancer, gastric cancer, esophageal cancer and pancreatic cancer.

Embodiment 93. The isolated antibody or antigen binding fragment thereof, or the pharmaceutical composition, for the use of any one of embodiments 90-92, wherein the cancer is or comprises colorectal cancer.

Embodiment 94. The isolated antibody or antigen binding fragment thereof, or the pharmaceutical composition, for the use of any one of embodiments 90-93, wherein the cancer is or comprises advanced or metastatic colorectal cancer.

Embodiment 95. The isolated antibody or antigen binding fragment thereof, or the pharmaceutical composition, for the use of embodiment 94, wherein the cancer is or comprises unresectable metastatic colorectal cancer.

Embodiment 96. The isolated antibody or antigen binding fragment thereof, or the pharmaceutical composition, for the use of any one of embodiments 90-92, wherein the cancer is or comprises gastric cancer.

Embodiment 97. The isolated antibody or antigen binding fragment thereof, or the pharmaceutical composition, for the use of any one of embodiments 90-92, wherein the cancer is or comprises esophageal cancer.

Embodiment 98. The isolated antibody or antigen binding fragment thereof, or the pharmaceutical composition, for the use of any one of embodiments 90-92, wherein the cancer is or comprises pancreatic cancer.

Embodiment 99. The isolated antibody or antigen binding fragment thereof, or the pharmaceutical composition for the use of any one of embodiments 70-98, which is part of a combination therapy

### EXAMPLES

### Example 1. Generation of Anti-GUCY2C Antibodies

### Immunization of Ablexis Kappa Mice

Ablexis Kappa mice were immunized with alternating boosts of 10 μg of cyno GUCY2C extracellular domain (ECD) (day 0, 14, 28) or human GUCY2C ECD (day 7, 21). Zinc chitosan nanoparticles were used in conjunction in some instances. The mice received 5 weekly boosts and a final boost at day 44 with human GUCY2C ECD (with zinc chitosan nanoparticles in some instances) and 50 μg of anti-mouse CD40 (R&D Systems MAB440).

Immune sera samples were collected once during the immunization (Day 28) and tested for binding via flow cytometry to T84 (GUCY2C+) and T84 GUCY2C KO (GUCY2C−) cells. Protein serology was done on human and cyno GUCY2C ECD proteins by MSD. Quality of Immune Response analysis was done by SPR on human and cyno GUCY2C ECD coated chips.

### Single B-Cell Recovery

On Day 51 of the immunization schedule, spleens and inguinal lymph nodes were harvested from each mouse of group 4. All lymph nodes were pooled and homogenized into a single-cell suspension, sorted on double positive AF647-labeled cyno GUCY2C and biotinylated human GUCY2C (with streptavidin-PE detection). Cells were then plated and cultured for 7 days.

Supernatants from the sorted B cells were screened by protein MSD on biotinylated human and cynomolgus monkey GUCY2C. Primary hits were sent for V gene recovery. Selected recovered hits were expressed as human IgG1.

### Expi293Small Scale Transfection and Purification

Antibodies identified from the immunization campaign were cloned and expressed as human IgG1 at 2 ml scale and purified. Expi293 cells were cultured in Expi293 Expression Medium at 37° C., 7% CO2. Cells were sub-cultured when density reached the log phase growth at 2.5-3×10\hat{ }6 viable cells/mL with a 98-99% viability. On the day of transfection, the viable cell density and percent viability was determined. Cells were transfected at a density of 2.5-3×10\hat{ }6 viable cells/mL following manufacturer's Transfection protocol (ThermoFisher ExpiCHO Expression System Protocols for 24 and 96 deep well blocks and mini bioreactor tubes). Culture supernatants were harvested on Day 5 post-transfection by centrifugation at 2000 RPM for 15 minutes prior to purification. Antibodies were purified from the clarified supernatants using Capture Select CH1-XL resin slurry (Thermo Cat 2943452050). Antibodies were eluted with 0.1 M Na-Acetate, pH 3.5 and each elution fraction was neutralized with 2.5 M Tris HC1, pH 7.5 before dialyzes into DPBS. Protein concentrations were determined by measurement of absorbance at 280 nm on the filtrate using a DropSense Instrument (Trinean NV/SA).

### Recombinant Antibody Screening

The antibodies were screened for binding on T84 cells, CHO-huGUCY2C and EL4-huGUCY2C and counter screened on untransfected EL4 in dose response titrations. The best binders were then rescreened in dose response titrations on CHO-huGUCY2C, CHO-cynoGUCY2C, untransfected CHO and EL-4 huGUCY2C. They were also tested for competition against index GUCY2C binders and a known GUCY2C binder (reference binder) for binding to CHO-huGUCY2C cells. The variable regions of selected antibodies were sequenced. A mAb called GUCYC2_mAb was selected for further studies.

### Sequences of GUCY2C_mAb

Sequences of representative GUCY2C_mAb are provided in Tables 5-8. Table 5 shows the HCDR1, HCDR2, HCDR3, LCDR1, LCDR2 and LCDR3 sequences of GUCY2C_mAb under the Kabat delineation. Table 6 shows the HCDR1, HCDR2, HCDR3, LCDR1, LCDR2 and LCDR3 sequences of GUCY2C_mAb under the Chothia delineation. Table 7 shows the HCDR1, HCDR2, HCDR3, LCDR1, LCDR2 and LCDR3 sequences of GUCY2C_mAb under the ABM delineation. Table 8 shows the HCDR1, HCDR2, HCDR3, LCDR1, LCDR2 and LCDR3 sequences of GUCY2C_mAb under the Contact delineation. Table 9 shows the HCDR1, HCDR2, HCDR3, LCDR1, LCDR2 and LCDR3 sequences of GUCY2C_mAb under the IMGT delineation.

TABLE 5
HCDRs and LCDRs of GUYC2C_mAb under the Kabat
delineation.

| CDR | Sequence | SEQ ID NO |
|---|---|---|
| HCDR1 | HYYWS | 1 |
| HCDR2 | RIYTSGSTNYNPSLKS | 2 |
| HCDR3 | DRYTGYFDL | 3 |
| LCDR1 | RASQSVSSSYLA | 4 |
| LCDR2 | GASSRAT | 5 |
| LCDR3 | QQYGSSPPT | 6 |

TABLE 6
HCDRs and LCDRs of GUYC2C_mAb under the Chothia
delineation.

| CDR | Sequence | SEQ ID NO |
|---|---|---|
| HCDR1 | GASISHY | 23 |
| HCDR2 | YTSGS | 24 |
| HCDR3 | DRYTGYFDL | 3 |
| LCDR1 | RASQSVSSSYLA | 4 |
| LCDR2 | GASSRAT | 5 |
| LCDR3 | QQYGSSPPT | 6 |

TABLE 7
HCDRs and LCDRs of GUYC2C_mAb under the AbM
delineation.

| CDR | Sequence | SEQ ID NO |
|---|---|---|
| HCDR1 | GASISHYYWS | 25 |
| HCDR2 | RIYTSGSTN | 26 |
| HCDR3 | DRYTGYFDL | 3 |
| LCDR1 | RASQSVSSSYLA | 4 |
| LCDR2 | GASSRAT | 5 |
| LCDR3 | QQYGSSPPT | 6 |

TABLE 8
HCDRs and LCDRs of GUYC2C_mAb under the Contact
delineation.

| CDR | Sequence | SEQ ID NO |
|---|---|---|
| HCDR1 | SHYYWS | 27 |
| HCDR2 | WIGRIYTSGSTN | 28 |
| HCDR3 | ARDRYTGYFD | 79 |
| LCDR1 | SSSYLAWY | 29 |
| LCDR2 | LLIYGASSRA | 30 |
| LCDR3 | QQYGSSPP | 31 |

TABLE 9
HCDRs and LCDRs of GUYC2C_mAb under the IMGT
delineation.

| CDR | Sequence | SEQ ID NO |
|---|---|---|
| HCDR1 | GASISHYY | 80 |
| HCDR2 | IYTSGST | 81 |
| HCDR3 | ARDRYTGYFDL | 82 |
| LCDR1 | QSVSSSY | 83 |
| LCDR2 | GAS | NA |
| LCDR3 | QQYGSSPPT | 6 |

Table 10 shows the VH1 and VL1 amino acid sequences of GUCY2C_mAb. Table 11 shows the VH1 and VL1 nucleic acid sequences of GUCY2C_mAb.

TABLE 10
Amino acid sequences of the VH1 and VL1 of GUCY2C_mAb

| | Sequence | SEQ ID NO: |
|---|---|---|
| VH1 | QVQLQESGPGLVKPSETLSLTCTVSGASISHYYWSWIRRPAG | 7 |
| | KGLEWIGRIYTSGSTNYNPSLKSRVTVSVDTSKNQFSLKLSSV | |
| | TAADTAVYYCARDRYTGYFDLWGRGTLVTVSS | |
| VL1 | EIVLTQSPGTLSLSPGERATLSCRASQSVSSSYLAWYQQKPGQ | 8 |
| | APRLLIYGASSRATGIPDRFSGSGSGTDFTLTISRLEPEDFAVY | |
| | YCQQYGSSPPTFGGGTKLEIK | |

TABLE 11
Nucleic acid sequences of the VH1 and VL1 of GUCY2C_mAb

| | Sequence | SEQ ID NO: |
|---|---|---|
| VH1 | CAGGTGCAGCTGCAGGAGTCGGGCCCAGGACTGGTGAAG | 32 |
| | CCTTCGGAGACCCTGTCCCTCACCTGCACTGTCTCTGGTGC | |
| | CTCCATCAGTCATTACTACTGGAGCTGGATCCGGCGGCCC | |
| | GCCGGGAAGGGACTGGAGTGGATTGGGCGTATCTATACCA | |
| | GTGGGAGCACCAACTACAACCCCTCCCTCAAGAGTCGAGT | |
| | CACCGTGTCAGTAGACACGTCCAAGAACCAGTTCTCCCTG | |
| | AAGCTGAGCTCTGTGACCGCCGCGGACACGGCCGTATATT | |
| | ACTGTGCGAGAGATCGATATACGGGGTACTTCGATCTCTG | |
| | GGGCCGTGGCACCCTGGTCACTGTCTCCTCA | |
| VL1 | GAAATTGTGTTGACGCAGTCTCCAGGCACCCTGTCTTTGTC | 33 |
| | TCCAGGGGAAAGAGCCACCCTCTCCTGCAGGGCCAGTCAG | |
| | AGTGTTAGCAGCAGCTACTTAGCCTGGTACCAGCAGAAAC | |
| | CTGGCCAGGCTCCCAGGCTCCTCATCTATGGTGCATCCAG | |
| | CAGGGCCACTGGCATCCCAGACAGGTTCAGTGGCAGTGGG | |
| | TCTGGGACAGACTTCACTCTCACCATCAGCAGACTGGAGC | |
| | CTGAAGATTTTGCAGTTTATTACTGTCAGCAGTATGGTAGC | |
| | TCACCTCCCACTTTCGGCGGAGGGACCAAGCTGGAGATCA | |
| | AA | |

### Example 2. Characterization of GUCY2C_mAb

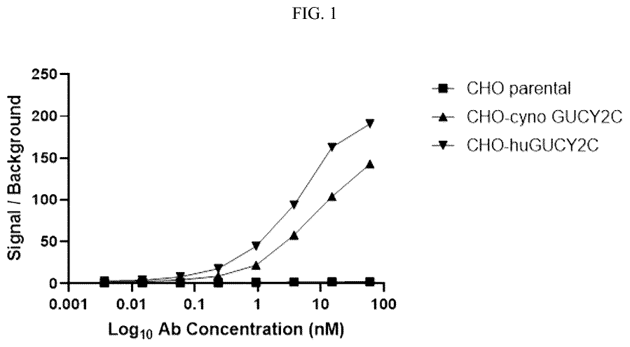

Three (3) cell lines, Chinese hamster ovary (CHO) cell line, CHO transfected with cyno GUCY2C, and CHO transfected with human GUCY2C, were incubated with a titrating dose of GUCY2C_mAb for 60 min at 37° C., washed twice, then incubated with AF647 labeled anti human secondary for 30 minutes at 4° C. Cells were washed twice again, resuspended and acquired in a flow cytometer. Specific antibody binding was assessed in the RL1H channel for each cell line. Data presented as signal/background where background is secondary only. As shown in FIG. 1, GUCY2C_mAb as generated herein binds to both human and cyno GUCY2C, with higher binding affinity to human CUCY2C.

EC50 values for binding of GUCY2C_mAb to CHO cells transfected with human or cyno GUCY2C is shown in Table 12.

TABLE 12

| Cells | EC50 |
|---|---|
| CHO-huGUCY2C | 4.6 nM |
| CHO-cynoGUCY2C | 8.7 nM |

### Example 3. Generation of GUCY2CxCD3 Bispecific Antibodies

### Engineering of GUCY2CxCD3 Bispecific Antibodies

In this example, two (2) GUCY2CxCD3 bispecific antibodies were generated (GUCY2CxCD3_biAb-1 and GUCY2CxCD3_biAb-2). GUCY2CxCD3_biAb-1 and GUCY2CxCD3_biAb-2 comprise the same GUCY2C binding arm, which corresponds to the GUCY2C_mAb described above and a medium and high affinity CD3 binding arm, respectively.

### Engineering of GUCY2CxCD3_biAb-1

Briefly, the GUCY2C binding arm of GUCY2CxCD3_biAb-1 comprises an anti-GUCY2C Fab (GUCY2C Fab) derived from GUCY2C_mAb describe in Example 1. To prepare the bispecific antibody, the VH1 and VL1 of GUCY2C_mAb were engineered in VH1-CH1-hinge-CH2-CH3 (Heavy Chain 1, HC1) and VL1-CL (Light Chain 1, LC1) formats respectively and expressed as IgG1. The Fc silencing mutation L234A/L235A/D265S were introduced in the Fc region of HC1. Mutations designed to promote selective heterodimerization (“hole” mutation T366S, L368A and Y407V) were also engineered in the Fc domain of HC1.

The CD3 binding arm of GUCY2CxCD3 biAb-1 comprises a stabilized (or stapled) scFv domain herein described as spFv-1. The CD3 arm of GUCY2CxCD3_biAb-1 is a low-medium affinity CD3 binding arm which will herein be referred to as CD3_spFv-1. The CD3_spFv-1 is derived from an anti-CD3 scFv fragment described in WO2022/201053, which is incorporated herein by reference in its entirety.

The anti-CD3 scFv as disclosed in WO2022/201053 was engineered into a stabilized scFv (herein referred to “stapled scFv” or spFv) by restraining the scFv structure, without negatively impacting, the relative movements between the VH2 and the VL2. The stabilized scFv was generated by engineering disulfide bonds between the VH2 and the linker and between the VL2 and the linker. Two structurally conserved surface exposed framework positions (anchor points) that are not involved in antigen binding, were identified, one on the VH2 at position H105 and one on the VL2 at position L42, and mutated into cysteine (Cys) residues to generate the VH2 of SEQ ID NO: 17 and VL2 of SEQ ID NO: 18. A flexible linker of sequence GGGSGGSGGCPPCGGSGG (SEQ ID NO: 40) comprising two Cys residues was used to conjugate the VL2 and the VH2 in the VL-linker-VH (LH) format. The distance and location of the Cys residues of the linker and the Cys residues of the VH2 and the VL2 is critical for the formation of the disulfide bonds between the Cys residues of the Linker and each anchor point of the VH2 and the VL2.

The stapled scFv in the VL-Linker-VH was further engineered into a Heavy Chain 2 (HC2) and expressed as IgG1. Additionally, the Fc silencing mutation L234A/L235A/D265S and the heterodimerization (“knob” mutation T366W) were engineered in the Fc domain of HC2.

HC2 comprising CD3_spFv-1 was then paired with HC1 and LC1 comprising the GUCY2C_Fab using the knob-in-hole heterodimerization technology to generate the stapled GUCY2CxCD3_biAb-1.

The affinity of CD3_spFv-1 to T cells was measured by flow cytometry based Intellicyt assay. Briefly, pan T cells were isolated from PBMC from healthy donors. Cells were incubated with titrating amounts test molecules for 1 hour, then washed. Bound antibodies were detected with Alexa Fluor 647 F(ab′)₂ fragment goat anti-human IgG. Cells were then washed and resuspended in 0.10% pluronic acid containing Sytox Green, then immediately analyzed in a flow cytometer. Median fluorescent intensity (MFI) was recorded (on RL1-A). The log 10 of the molecule concentration was plotted against the MFI. Best fit curves were evaluated using a 4PL algorithm (log(agonist) vs. response—Variable slope) in GraphPad Prism 9.0. 50% effective concentrations (EC50) values were derived from the best-fit curves. The EC50 value of CD3_spFv-1 for human CD3 is 108-112 nM.

CDR sequences, VH2, VL2 and spFv sequence of CD3_spFv-1 are shown in Table 13-15.

TABLE 13
HCDRs and LCDRs of CD3_spFv-1 under the Kabat
delineation.

| CDR | Sequence | SEQ ID NO |
|---|---|---|
| HCDR1 | RSTMH | 11 |
| HCDR2 | YINPSSAYTNYNQKFQG | 12 |
| HCDR3 | PQVHYDYAGFPY | 13 |
| LCDR1 | SASSSVSYMN | 14 |
| LCDR2 | DSSKLAS | 15 |
| LCDR3 | QQWSRNPPT | 16 |

TABLE 14
Amino Acid sequences of VH2, VL2 and stapled VL-Linker-VH of CD3_spFv-1

| | Sequence | SEQ ID NO: |
|---|---|---|
| VH2 | QVQLVQSGAEVKKPGSSVKVSCKASGYTFTRSTM | 17 |
| | HWVKQAPGQGLEWIGYINPSSAYTNYNQKFQGRV | |
| | TLTADKSTSTAYMELSSLRSEDTAVYYCASPQVHY | |
| | DYAGFPYWGCGTLVTVSS | |
| VL2 | EIVLTQSPATLSASPGERVTLSCSASSSVSYMNWY | 18 |
| | QQKPGCAPRRWIYDSSKLASGVPARFSGSGSGRDY | |
| | TLTISSLEPEDFAVYYCQQWSRNPPTFGGGTKVEIK | |
| Stapled VL2-Linker- | EIVLTQSPATLSASPGERVTLSCSASSSVSYMNWY | 22 |
| VH2 | QQKPGCAPRRWIYDSSKLASGVPARFSGSGSGRDY | |
| | TLTISSLEPEDFAVYYCQQWSRNPPTFGGGTKVEIK | |
| | GGGSGGSGGCPPCGGSGGQVQLVQSGAEVKKPGS | |
| | SVKVSCKASGYTFTRSTMHWVKQAPGQGLEWIGY | |
| | INPSSAYTNYNQKFQGRVTLTADKSTSTAYMELSS | |
| | LRSEDTAVYYCASPQVHYDYAGFPYWGCGTL | |
| | VTVSS | |

TABLE 15
Nucleotide sequences of VH2, VL2 and stapled VL-Linker-VH of CD3_spFv-1

| | sequence | SEQ ID NO: |
|---|---|---|
| VH2 | CAAGTACAACTTGTTCAATCTGGCGCTGAAGTCA | 34 |
| | AAAAACCCGGATCTAGTGTTAAAGTTTCATGTA | |
| | AGGCATCAGGCTATACTTTCACTCGGTCTACAAT | |
| | GCACTGGGTCAAGCAGGCTCCAGGGCAAGGTTT | |
| | GGAATGGATAGGGTACATAAATCCTTCTAGCGC | |
| | CTATACTAATTACAACCAGAAGTTTCAGGGACG | |
| | TGTTACTCTGACTGCTGACAAATCAACATCTACA | |
| | GCCTATATGGAACTGTCTTCATTGAGGAGTGAG | |
| | GACACTGCTGTGTATTACTGCGCTTCTCCTCAGG | |
| | TGCACTATGATTACGCTGGCTTTCCCTACTGGGG | |
| | TTGTGGCACCCTCGTCACCGTTTCATCT | |
| VL2 | GAGATCGTCTTGACTCAGTCCCCTGCAACCCTCA | 35 |
| | GCGCAAGTCCCGGAGAGCGCGTTACATTGAGTT | |
| | GTTCCGCATCCAGTTCAGTCAGCTACATGAACTG | |
| | GTACCAACAAAAGCCCGGTTGTGCTCCAAGAAG | |
| | GTGGATCTATGATTCCAGTAAGCTGGCCTCCGGG | |
| | GTCCCAGCAAGGTTCAGCGGTTCCGGCAGCGGA | |
| | AGGGACTACACTCTGACCATAAGTTCCCTTGAAC | |
| | CAGAAGACTTCGCCGTCTATTATTGTCAGCAGTG | |
| | GTCCCGAAATCCACCTACCTTCGGGGGCGGCAC | |
| | TAAAGTGGAGATTAAA | |
| Stapled VL2-Linker- | GAGATCGTCTTGACTCAGTCCCCTGCAACCCTCA | 36 |
| VH2 | GCGCAAGTCCCGGAGAGCGCGTTACATTGAGTT | |
| | GTTCCGCATCCAGTTCAGTCAGCTACATGAACTG | |
| | GTACCAACAAAAGCCCGGTTGTGCTCCAAGAAG | |
| | GTGGATCTATGATTCCAGTAAGCTGGCCTCCGGG | |
| | GTCCCAGCAAGGTTCAGCGGTTCCGGCAGCGGA | |
| | AGGGACTACACTCTGACCATAAGTTCCCTTGAAC | |
| | CAGAAGACTTCGCCGTCTATTATTGTCAGCAGTG | |
| | GTCCCGAAATCCACCTACCTTCGGGGGCGGCAC | |
| | TAAAGTGGAGATTAAAGGAGGAGGTAGTGGCGG | |
| | CTCCGGTGGATGTCCTCCTTGCGGAGGATCAGG | |
| | AGGTCAAGTACAACTTGTTCAATCTGGCGCTGA | |
| | AGTCAAAAAACCCGGATCTAGTGTTAAAGTTTC | |
| | ATGTAAGGCATCAGGCTATACTTTCACTCGGTCT | |
| | ACAATGCACTGGGTCAAGCAGGCTCCAGGGCAA | |
| | GGTTTGGAATGGATAGGGTACATAAATCCTTCTA | |
| | GCGCCTATACTAATTACAACCAGAAGTTTCAGG | |
| | GACGTGTTACTCTGACTGCTGACAAATCAACATC | |
| | TACAGCCTATATGGAACTGTCTTCATTGAGGAGT | |
| | GAGGACACTGCTGTGTATTACTGCGCTTCTCCTC | |
| | AGGTGCACTATGATTACGCTGGCTTTCCCTACTG | |
| | GGGTTGTGGCACCCTCGTCACCGTTTCATCT | |

The full-length amino acid sequences of GUCY2C-CD3-bisp-Abl are shown in Table 16-17. Table 16 shows the full-length amino acid sequences of the GUCY2C arm (HC1 and LC1) and of the CD3 arm (HC2). Table 17 shows the nucleic acid sequences of the GUCY2C arm (HC1 and LC1) and of the CD3 arm (HC2).

TABLE 16
Full length amino acid sequence of GUCY2CxCD3 bisp-Ab1

| | Sequence | SEQ ID NO: |
|---|---|---|
| HC1 (GUCY2C | QVQLQESGPGLVKPSETLSLTCTVSGASISHYYWSWIRRPAG | 9 |
| binding arm) | KGLEWIGRIYTSGSTNYNPSLKSRVTVSVDTSKNQFSLKLSS | |
| | VTAADTAVYYCARDRYTGYFDLWGRGTLVTVSSASTKGPS | |
| | VFPLAPSSKSTSGGTAALGCLVKDYFPEPVTVSWNSGALTS | |
| | GVHTFPAVLQSSGLYSLSSVVTVPSSSLGTQTYICNVNHKPS | |
| | NTKVDKKVEPKSCDKTHTCPPCPAPEAAGGPSVFLFPPKPK | |
| | DTLMISRTPEVTCVVVSVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLSCAVK | |
| | GFYPSDIAVEWESNGQPENNYKTTPPVLDSDGSFFLVSKLTV | |
| | DKSRWQQGNVFSCSVMHEALHNRFTQKSLSLSPGK | |
| LC1 (GUCY2C | EIVLTQSPGTLSLSPGERATLSCRASQSVSSSYLAWYQQKPG | 10 |
| binding arm) | QAPRLLIYGASSRATGIPDRFSGSGSGTDFTLTISRLEPEDFA | |
| | VYYCQQYGSSPPTFGGGTKLEIKRTVAAPSVFIFPPSDEQLK | |
| | SGTASVVCLLNNFYPREAKVQWKVDNALQSGNSQESVTEQ | |
| | DSKDSTYSLSSTLTLSKADYEKHKVYACEVTHQGLSSPVTK | |
| | SFNRGEC | |
| HC2 (CD3 | EIVLTQSPATLSASPGERVTLSCSASSSVSYMNWYQQKPGC | 19 |
| binding arm) | APRRWIYDSSKLASGVPARFSGSGSGRDYTLTISSLEPEDFA | |
| | VYYCQQWSRNPPTFGGGTKVEIKGGGSGGSGGCPPCGGSG | |
| | GQVQLVQSGAEVKKPGSSVKVSCKASGYTFTRSTMHWVK | |
| | QAPGQGLEWIGYINPSSAYTNYNQKFQGRVTLTADKSTSTA | |
| | YMELSSLRSEDTAVYYCASPQVHYDYAGFPYWGCGTLVTV | |
| | SSEPKSSDKTHTCPPCPAPEAAGGPSVFLFPPKPKDTLMISRT | |
| | PEVTCVVVSVSHEDPEVKFNWYVDGVEVHNAKTKPREEQY | |
| | NSTYRVVSVLTVLHQDWLNGKEYKCKVSNKALPAPIEKTIS | |
| | KAKGQPREPQVYTLPPSREEMTKNQVSLWCLVKGFYPSDIA | |
| | VEWESNGQPENNYKTTPPVLDSDGSFFLYSKLTVDKSRWQ | |
| | QGNVFSCSVMHEALHNHYTQKSLSLSPGK | |

TABLE 17
Full length nucleic acid sequence of GUCY2CxCD3 bisp-Ab1

| | Sequence | SEQ ID NO: |
|---|---|---|
| HC1 (GUCY2C | CAGGTGCAGCTGCAGGAGTCGGGCCCAGGACTGGTGAAG | 37 |
| binding arm) | CCTTCGGAGACCCTGTCCCTCACCTGCACTGTCTCTGGTG | |
| | CCTCCATCAGTCATTACTACTGGAGCTGGATCCGGCGGCC | |
| | CGCCGGGAAGGGACTGGAGTGGATTGGGCGTATCTATAC | |
| | CAGTGGGAGCACCAACTACAACCCCTCCCTCAAGAGTCG | |
| | AGTCACCGTGTCAGTAGACACGTCCAAGAACCAGTTCTC | |
| | CCTGAAGCTGAGCTCTGTGACCGCCGCGGACACGGCCGT | |
| | ATATTACTGTGCGAGAGATCGATATACGGGGTACTTCGA | |
| | TCTCTGGGGCCGTGGCACCCTGGTCACTGTCTCCTCAGCC | |
| | TCCACCAAGGGCCCATCGGTCTTCCCCCTGGCACCCTCCT | |
| | CCAAGAGCACCTCTGGGGGCACAGCGGCCCTGGGCTGCC | |
| | TGGTCAAGGACTACTTCCCCGAACCGGTGACGGTGTCGT | |
| | GGAACTCAGGCGCCCTGACCAGCGGCGTGCACACCTTCC | |
| | CGGCTGTCCTACAGTCCTCAGGACTCTACTCCCTCAGCAG | |
| | CGTGGTGACCGTGCCCTCCAGCAGCTTGGGCACCCAGAC | |
| | CTACATCTGCAACGTGAATCACAAGCCCAGCAACACCAA | |
| | GGTGGACAAGAAAGTTGAGCCCAAATCTTGTGACAAAAC | |
| | TCACACATGTCCACCGTGCCCAGCACCTGAAGCAGCAGG | |
| | GGGACCGTCAGTCTTCCTCTTCCCCCCAAAACCCAAGGAC | |
| | ACCCTCATGATCTCCCGGACCCCTGAGGTCACATGCGTGG | |
| | TGGTGAGCGTGAGCCACGAAGACCCTGAGGTCAAGTTCA | |
| | ACTGGTACGTGGACGGCGTGGAGGTGCATAATGCCAAGA | |
| | CAAAGCCGCGGGAGGAGCAGTACAACAGCACGTACCGT | |
| | GTGGTCAGCGTCCTCACCGTCCTGCACCAGGACTGGCTG | |
| | AATGGCAAGGAGTACAAGTGCAAGGTCTCCAACAAAGCC | |
| | CTCCCAGCCCCCATCGAGAAAACCATCTCCAAAGCCAAA | |
| | GGGCAGCCCCGAGAACCACAGGTGTACACCCTGCCCCCA | |
| | TCCCGGGAGGAGATGACCAAGAACCAGGTCAGCCTGTCC | |
| | TGCGCGGTCAAAGGCTTCTATCCCAGCGACATCGCCGTG | |
| | GAGTGGGAGAGCAATGGGCAGCCGGAGAACAACTACAA | |
| | GACCACGCCTCCCGTGCTGGACTCCGACGGCTCCTTCTTC | |
| | CTCGTCAGCAAGCTCACCGTGGACAAGTCTAGATGGCAG | |
| | CAGGGGAACGTCTTCTCATGCTCCGTGATGCATGAGGCTC | |
| | TGCACAACCGGTTCACGCAGAAGAGCCTCTCCCTGT | |
| | CTCCGGGTAAA | |
| LC1 (GUCY2C | GAAATTGTGTTGACGCAGTCTCCAGGCACCCTGTCTTTGT | 38 |
| binding arm) | CTCCAGGGGAAAGAGCCACCCTCTCCTGCAGGGCCAGTC | |
| | AGAGTGTTAGCAGCAGCTACTTAGCCTGGTACCAGCAGA | |
| | AACCTGGCCAGGCTCCCAGGCTCCTCATCTATGGTGCATC | |
| | CAGCAGGGCCACTGGCATCCCAGACAGGTTCAGTGGCAG | |
| | TGGGTCTGGGACAGACTTCACTCTCACCATCAGCAGACT | |
| | GGAGCCTGAAGATTTTGCAGTTTATTACTGTCAGCAGTAT | |
| | GGTAGCTCACCTCCCACTTTCGGCGGAGGGACCAAGCTG | |
| | GAGATCAAACGTACGGTGGCTGCACCATCTGTCTTCATCT | |
| | TCCCGCCATCTGATGAGCAGTTGAAATCTGGAACTGCCTC | |
| | TGTTGTGTGCCTGCTGAATAACTTCTATCCCAGAGAGGCC | |
| | AAAGTACAGTGGAAGGTGGATAACGCCCTCCAATCGGGT | |
| | AACTCCCAGGAGAGTGTCACAGAGCAGGACAGCAAGGA | |
| | CAGCACCTACAGCCTCAGCAGCACCCTGACGCTGAGCAA | |
| | AGCAGACTACGAGAAACACAAAGTCTACGCCTGCGAAGT | |
| | CACCCATCAGGGCCTGAGCTCGCCCGTCACAAAGAGCTT | |
| | CAACAGGGGAGAGTGT | |
| HC2 (CD3 | GAGATCGTCTTGACTCAGTCCCCTGCAACCCTCAGCGCAA | 39 |
| binding arm) | GTCCCGGAGAGCGCGTTACATTGAGTTGTTCCGCATCCAG | |
| | TTCAGTCAGCTACATGAACTGGTACCAACAAAAGCCCGG | |
| | TTGTGCTCCAAGAAGGTGGATCTATGATTCCAGTAAGCTG | |
| | GCCTCCGGGGTCCCAGCAAGGTTCAGCGGTTCCGGCAGC | |
| | GGAAGGGACTACACTCTGACCATAAGTTCCCTTGAACCA | |
| | GAAGACTTCGCCGTCTATTATTGTCAGCAGTGGTCCCGAA | |
| | ATCCACCTACCTTCGGGGGCGGCACTAAAGTGGAGATTA | |
| | AAGGAGGAGGTAGTGGCGGCTCCGGTGGATGTCCTCCTT | |
| | GCGGAGGATCAGGAGGTCAAGTACAACTTGTTCAATCTG | |
| | GCGCTGAAGTCAAAAAACCCGGATCTAGTGTTAAAGTTT | |
| | CATGTAAGGCATCAGGCTATACTTTCACTCGGTCTACAAT | |
| | GCACTGGGTCAAGCAGGCTCCAGGGCAAGGTTTGGAATG | |
| | GATAGGGTACATAAATCCTTCTAGCGCCTATACTAATTAC | |
| | AACCAGAAGTTTCAGGGACGTGTTACTCTGACTGCTGAC | |
| | AAATCAACATCTACAGCCTATATGGAACTGTCTTCATTGA | |
| | GGAGTGAGGACACTGCTGTGTATTACTGCGCTTCTCCTCA | |
| | GGTGCACTATGATTACGCTGGCTTTCCCTACTGGGGTTGT | |
| | GGCACCCTCGTCACCGTTTCATCTGAGCCCAAATCTAGCG | |
| | ACAAAACTCACACATGTCCACCGTGCCCAGCACCTGAAG | |
| | CAGCAGGGGGACCGTCAGTCTTCCTCTTCCCCCCAAAACC | |
| | CAAGGACACCCTCATGATCTCCCGGACCCCTGAGGTCAC | |
| | ATGCGTGGTGGTGAGCGTGAGCCACGAAGACCCTGAGGT | |
| | CAAGTTCAACTGGTACGTGGACGGCGTGGAGGTGCATAA | |
| | TGCCAAGACAAAGCCGCGGGAGGAGCAGTACAACAGCA | |
| | CGTACCGTGTGGTCAGCGTCCTCACCGTCCTGCACCAGGA | |
| | CTGGCTGAATGGCAAGGAGTACAAGTGCAAGGTCTCCAA | |
| | CAAAGCCCTCCCAGCCCCCATCGAGAAAACCATCTCCAA | |
| | AGCCAAAGGGCAGCCCCGAGAACCACAGGTGTACACCCT | |
| | GCCCCCATCCCGGGAGGAGATGACCAAGAACCAGGTCAG | |
| | CCTGTGGTGCCTGGTCAAAGGCTTCTATCCCAGCGACATC | |
| | GCCGTGGAGTGGGAGAGCAATGGGCAGCCGGAGAACAA | |
| | CTACAAGACCACGCCTCCCGTGCTGGACTCCGACGGCTC | |
| | CTTCTTCCTCTACAGCAAGCTCACCGTGGACAAGTCTAGA | |
| | TGGCAGCAGGGGAACGTCTTCTCATGCTCCGTGATGCAT | |
| | GAGGCTCTGCACAACCACTACACGCAGAAGAGCCTCTCC | |
| | CTGTCTCCGGGTAAA | |

Engineering of GUCY2CxCD3 biAb-2

The GUCY2C binding arm of GUCY2CxCD3_biAb-2 is the same as for GUCY2CxCD3_biAb-1 described above.

The CD3 binding arm of GUCY2CxCD3_biAb-2 is a high affinity CD3 binding arm in a spFv format, and will herein be referred to as anti-CD3 spFv-2 (CD3_spFv-2). The affinity of CD3_spFv-2 for human CD3 was measured by flow cytometry based Intellicyt assay as described above. The calculated EC50 of CD3_spFv-2 for human CD3 is 5-19 nM.

The stapled scFv in the VL-Linker-VH was further engineered into a Heavy Chain 2 (HC2) and expressed as IgG1 comprising the Fc silencing mutation L234A/L235A/D265S and the heterodimerization (“knob” mutation T366W) in the Fc domain. HC2 comprising CD3_spFv-2 was then paired with the HC1 and the LC1 comprising the GUCY2C_Fab using the knob-in-hole heterodimerization technology to generate the stapled GUCY2CxCD3_biAb-2.

### Expression of GUCY2CxCD3 Antibodies

The GUCY2CxCD3 bispecific antibodies were expressed in ExpiCHO-S cells (ThermoFisher; Cat #A29127) by transient transfection with purified plasmid DNAs encoding LC1, HC1, and HC2 using the ExpiFectamine CHO transfection kit (ThermoFisher; Cat #A29131). ExpiCHO-S cells were maintained in suspension in ExpiCHO Expression Medium (ThermoFisher; Cat #A2910001) in an orbital shaking incubator set to 37° C., 8% CO2 and 125 rpm. The cells were passaged into 4 sterile, vented, non-baffled Erlenmeyer flasks (Corning 431255) with 400 mL starting culture volume per flask.

On the day of transfection (Culture Day 0), the flask cultures were between 5.15-8.99×10\hat{ }6 viable cells/mL with a minimum viability of 98.2%. For the transfection, 200 μg of plasmid DNA encoding HC2, 200 μg plasmid DNA encoding HC1, and 400 μg plasmid DNA encoding LC1 (HC2:HC1:LC1 chain ratio at 1:1:2) were diluted in 64 mL of OptiPRO medium (ThermoFisher Cat #12309019). 5.12 mL of ExpiFectamine CHO transfection reagent was diluted in another 64 mL of OptiPRO medium. The plasmid DNA plus OptiPRO medium mixture and the ExpiFectamine CHO reagent plus OptiPRO medium mixture were combined and incubated at room temperature for one minute. This was then equivalently distributed across the 4 flasks of cells, ˜33 mL/flask. The transfected cells were returned to the orbital shaking incubator.

After overnight incubation (Culture Day 1), 240 mL of ExpiCHO Feed and 4.8 mL ExpiFectamine CHO Enhancer were combined and then distributed equivalently across the 4 flasks. Cells were further incubated until harvest (Culture Day 7). The culture supernatant from the transiently transfected cells (6.06×10\hat{ }6⁶ viable cells/mL, 84.7% viability) was clarified by centrifugation (15 min, 5000 rpm (5316 RCF)) followed by 0.2 μm filtration.

### Purification of GUCY2CxCD3 Antibodies

The GUCY2CxCD3 bispecific antibodies were purified as follows. The filtered cell culture supernatant as prepared above was loaded onto a pre-equilibrated (1×DPBS, pH 7.2) custom 60 mL MabSelect PrismA column at 20 mL/min using an AKTA Pure150. After loading, the column was washed with 1× DPBS, pH 7.2 until UV stabilized near baseline. The protein was eluted with 0.1 M sodium acetate, pH 3.45, and neutralized inline by the addition of 2.5 M Tris HC1, pH 7.5 to 10% (v/v) final volume, filtered (0.2 μm), and dialyzed into 20 mM MES, pH 5.5. The dialyzed eluate was further purified by cation exchange chromatography (CEX) using a custom Capto S ImpAct column (GE Healthcare, 2.6 cm×36 cm, CV=193 mL) pre-equilibrated into buffer A (20 mM MES, pH 6.5). The protein was loaded at 10 mE/min and then eluted from the column with a gradient of buffer B (20 mM MES, pH 6.5, 1 M NaCl: 0-5% buffer B over 10 min, 5-35% buffer B over 135 min). The peak fractions containing only monomeric protein were pooled, dialyzed into 1× dPBS, PH 7.2 and filtered (0.2 μm).

### Example 4. Characterization of GUCY2CxCD3 Antibodies

### Tumor Cell Binding Specificity and T-Cell Binding Specificity

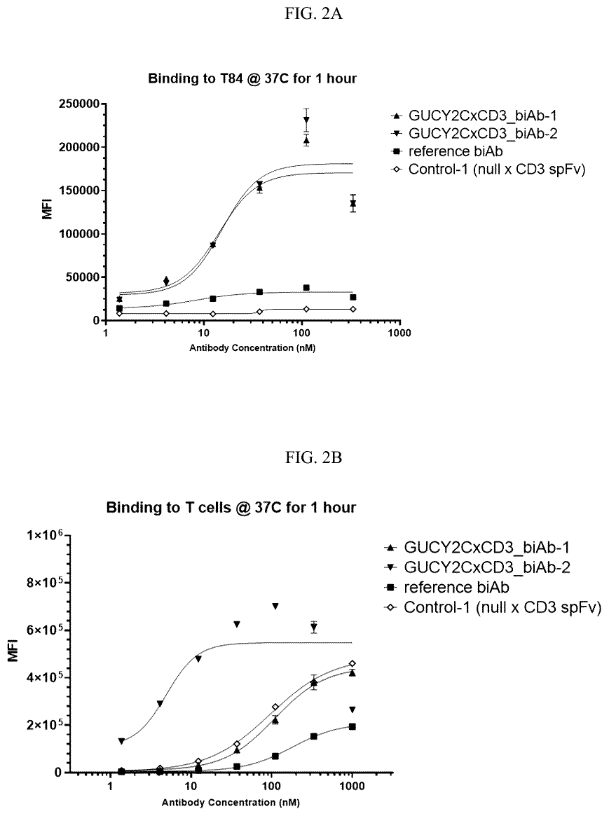

One hour cell binding studies by flow cytometry were carried out at physiologically relevant temperature (37° C.) on GUCY2CxCD3 bispecific antibodies (GUCY2CxCD3 biAb-1 and GUCY2CxCD3_biAb-2), known reference GUCY2CxCD3 bispecific antibody (biAb), and Null×CD3 control molecules. The goal was to characterize and quantify specific, cell binding for GUCY2CxCD3_biAb-1 and 2. As shown in FIG. 2A, GUCY2CxCD3_biAb-1 and 2 displayed, strong, specific binding to the GUCY2C endogenously expressing cell line T84. Maximum binding of GUCY2CxCD3 biAb-1 and 2 was higher than reference biAb. The Null×CD3 control did not bind to T84.

GUCY2CxCD3_biAb-1 bound to primary T cells to a similar extent as the Null×CD3, suggesting no impact of the GUCY2C Fab binding arm on T-cell engagement. GUCY2CxCD3 biAb-2, bearing a higher affinity CD3 binder, bound primary T cells with higher potency than GUCY2CxCD3-biAb-1 or Null×CD3 (FIG. 2B). Reference biAb also bound T cells as expected.

EC50 values for binding of GUCY2CxCD3_biAb-1 and 2 to T84 cells and T cells are summarized in Table 18.

TABLE 18

| | | |
|---|---|---|
| Bispecific antibody | EC50 to T84 | EC50 to T cells |

| | | | | |
|---|---|---|---|---|
| GUCY2CxCD3 biAb-1 | 12 | nM | 108 | nM |
| GUCY2CxCD3 biAb-2 | 12 | nM | 4.9 | nM |
| Reference biAb | 4.6 | nM | 180 | nM |

### Epitope Comparison

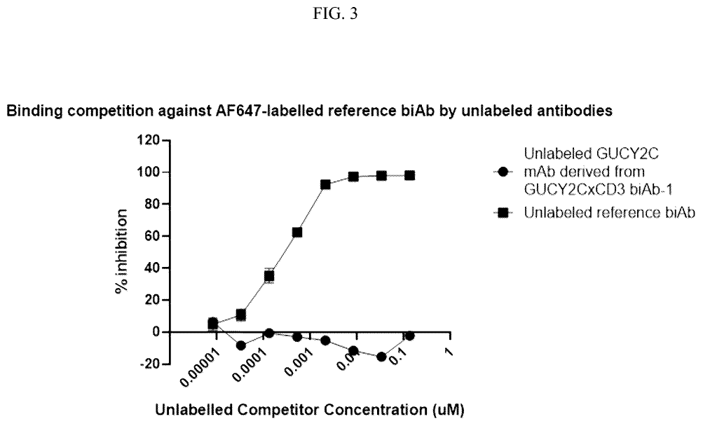

To determine whether the GUCY2C binder in GUCY2CxCD3_biAb-1 and 2 share the same epitope as reference biAb, competition flow cytometry experiments were performed using parental GUCY2C mAbs from which GUCY2CxCD3_biAb-1, GUCY2CxCD3_biAb-2 and reference Ab were derived. To that end, human GUCY2C transfected CHO cells were stained with AF647-labeled reference mAb in conjunction with a titration of unlabeled GUCY2C mAb from which GUCY2CxCD3 biAb-1 and 2 are derived. Plates were then washed and bound AF647-labeled reference mAb was then detected in a flow cytometer. As shown in FIG. 3, unlabeled GUCY2C mAb did not compete with AF647-labeled reference mAb for binding, while unlabeled reference biAb did. This indicates that the epitope of the GUCY2C binder in GUCY2CxCD3 biAb-1 and 2 is distinct from the epitope of the reference mAb.

### In Vitro Killing and T-Cell Activation

Impedance-based cytotoxicity assays performed on the RTCA xCELLigence (Agilent) platform were the primary means used to characterize the in vitro potency of GUCY2CxCD3 bispecific antibodies (GUCY2CxCD3 biAb-1 and GUCY2CxCD3_biAb-2), reference GUCY2CxCD3 bispecific antibody (biAb), and Null×CD3 control molecules. Experiments conducted on the IncuCyte® S3 (Sartorius) real-time, live cell imaging system were investigated as an orthogonal readout. CRC cell lines HT55, LS1034, and T84 were tested. They express GUCY2C at a range of 15000-40000 receptors/cell, characterized as a medium level of GUCY2C. To best approximate physiological relevance in the tumor microenvironment (TME), the lowest E:T ratios where maximal cytotoxicity was reached were used for lead identification and characterization.

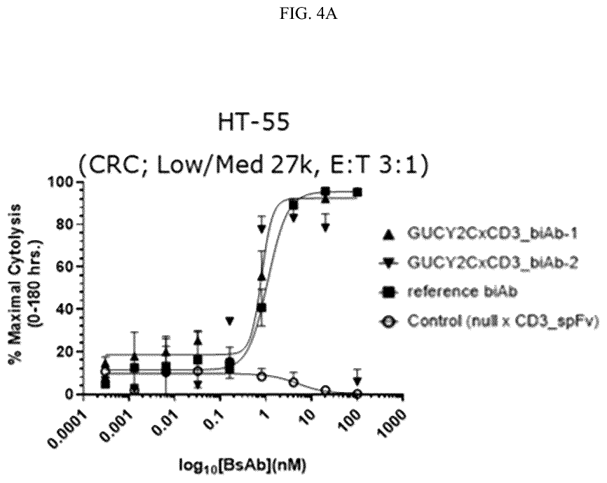

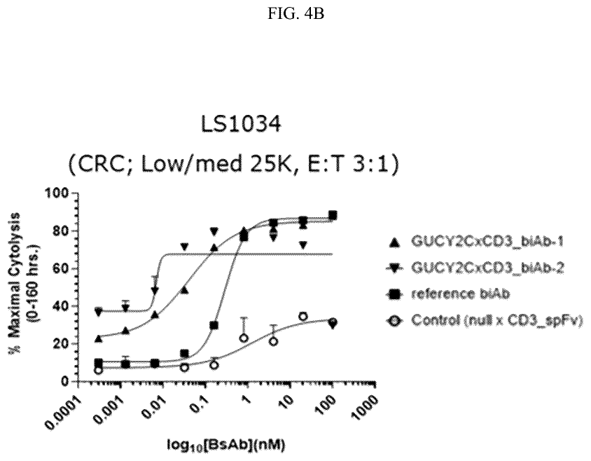

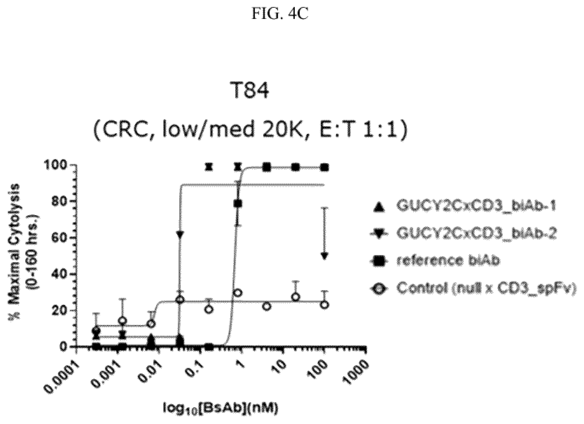

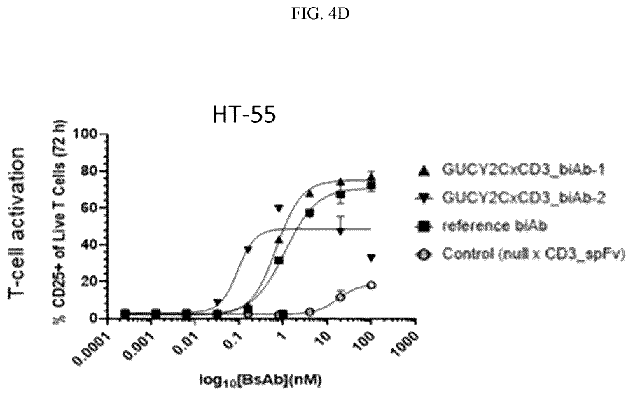

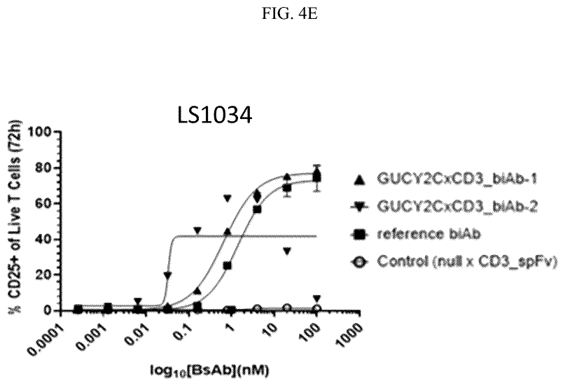

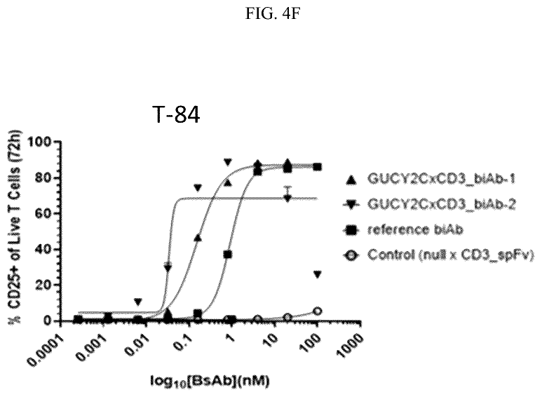

As shown in FIG. 4A-FIG. 4F, GUCY2CxCD3_biAb-1 induced T-cell mediated cytotoxicity and T-cell activation at an E:T ratio of 1:1 for T84 and a E:T ratio of 3:1 for both HT55 and LS1034 cells. GUCY2CxCD3_biAb-2 also induced T-cell mediated cytotoxicity and T-cell activation against these cell lines, but showed a hook effect at high concentrations. The reference biAb showed weaker T-cell mediated cytotoxicity and T-cell activation than both GUCY2CxCD3_biAb-1 and 2. The control nullxCD3 spFv did not show significant T-cell mediated cytotoxicity or T-cell activation against these cell lines.

EC50 values are shown in Table 19.

TABLE 19
Tumor killing EC50 (nM)

| Ab name | HT-55 | LS1034 | T84 |
|---|---|---|---|
| GUCY2CxCD3-biAb-1 | 0.8 | 0.04 | n/a |
| GUCY2CxCD3-biAb-2 | n/a | 0.007 | 0.03 |
| Reference biAb | 1.1 | 0.3 | 0.7 |

Abbreviations: n/a = not applicable because curve fit was ambiguous and did not allow for accurate calculation of EC50 values

### Anti-Tumor Efficacy in a T-Cell Humanized HT55 CDX Model

The antitumor activity and PD relationship of GUCY2CxCD3_biAb-1 was evaluated in the established SC human colorectal HT55 Cell-line Derived Xenograft (CDX) models in female immune-compromised NSG (ie, non-obese diabetic [NOD] severe combined immunodeficiency [scid] gamma or NOD.Cg-Prkd^{scid}Il2rg^{tm1Wjl}/SzJ) mice humanized with human donor CD3⁺ pan-T cells.

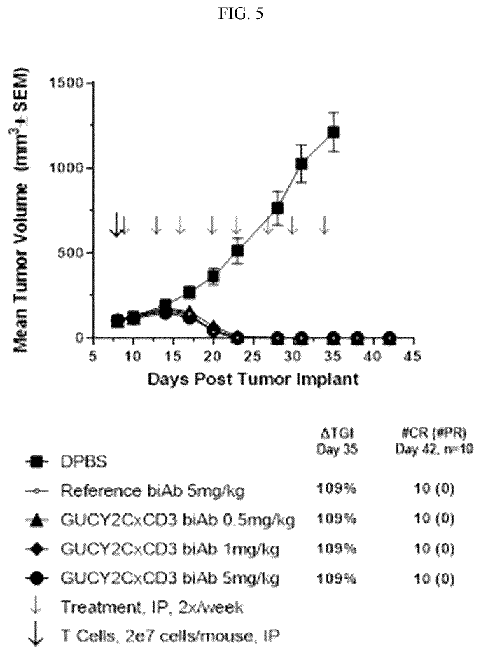

Mice bearing established SC HT55 xenografts were intraperitoneally (IP) dosed with GUCY2CxCD3 biAb-1 twice weekly at 0.5, 1 and 5 mg/kg or DPBS for a total of 8 doses (n=10/group). Significant antitumor efficacy was observed with GUCY2CxCD3 biAb-1 treatment at 0.5, 1, and 5 mg/kg with 109% A tumor growth inhibition (TGI) at all 3 dose levels (p<0.0001; FIG. 5), as compared to the DPBS-treated control group on Day 33 post tumor implantation. This resulted in 100% tumor regression (TR) and 10 of 10 complete regressions (CRs) in each treatment group by the end of the study.

### Conformational Stability

The thermal stability of the GUCY2CxCD3_biAb-1 was characterized by capillary VP-DSC microcalorimeter (Malvern Panalytical Malvern, UK). Temperature scans were performed from 25 to 100° C. in duplicate. A buffer reference scan was subtracted from protein scan and the concentration of protein was normalized prior to thermodynamic analysis. The data were plotted and analyzed using MicroCal PEAQ software. The DSC curve was fitted using non-two-state model to obtain the enthalpy and apparent transition temperature (Tm) values. GUCY2CxCD3_biAb-1 showed good thermal stability (Table 20).

TABLE 20

| | |
|---|---|
| Tonset | 62.7° C. |
| Tms (Tm1, Tm2, Tm3) | 71.8, 76.3 and 90.7° C. |

### Example 5. Exploratory Toxicity Study

As shown in example 2, the GUCY2C arm of GUCY2CxCD3-biAb-1 is cynomolgus monkey cross reactive but its CD3 bind arm is human specific and lack cross reactive to monkey. A GUCY2CxCD3 bispecific (GUCY2CxCD3_biAb-3) with tissue cross-reactivity in cynomolgus monkey was therefore prepared for this study. GUCY2CxCD3-biAb-3 was prepared as described above for GUCY2CxCD3_biAb-1 and 2. GUCY2CxCD3_biAb-3, comprises the same GUCY2C Fab arm (HC1 and LC1) as GUCY2CxCD3_biAb-1 and 2 but comprises a different CD3 binding arm also in a spFv format but with cross reactivity to cynomolgus monkey CD3. The CD3 arm of GUCY2CxCD3-biAb-3 is referred herein as CD3_spFv-3. The CDR sequences of CD3_spFv-3 are listed in Table 21. The affinity of CD3_spFv-3 for human and cynomolgus monkey CD3 was determined by cell-based assay as described in Example 3 for CD3_spFv-1 and CD3_spFv-2. The calculated EC50 of CD3_spFv-3 for human and cynomolgus monkey CD3 is 4.5 nM and 5 nM, respectively.

TABLE 21
HCDRs and LCDRs of CD3_spFv-3 under the Kabat
delineation

| Description | Sequences | SEQ ID NO |
|---|---|---|
| HCDR1 | TYAMN | 84 |
| HCDR2 | RIRSKYNNYATYYAASVKG | 85 |
| HCDR3 | HGNFGNSYVSWFAY | 86 |
| LCDR1 | RSSTGAVTTSNYAN | 87 |
| LCDR2 | GTNKRAP | 88 |
| LCDR3 | ALWYSNLWV | 89 |

The binding affinity of the cynomolgus monkey cross reactive CD3 arm (CD3_spFv-3) of GUCY2CxCD3-biAb-3 to cynomolgus CD3 is 2 to 20 times higher than that of the CD3_spFv-1 of GUCY2CxCD3-biAb-1 to human CD3. In one monkey T cell donor, the binding affinity of the CD3 arm of the GUCY2CxCD3-biAb-3 for monkey CD3 was 6 times higher than the binding affinity of the CD3 arm of the GUCY2CxCD3-biAb-1 for human CD3 in a human T cell donor. The higher CD3 binding affinity may result in higher level of cytokine release than would otherwise be observed with GUCY2CxCD3-biAb-1. GUCY2CxCD3-biAb-3 remains however an appropriate surrogate molecule in cynomolgus monkey to model the pharmacology and any associated potential toxicity resulting from GUCY2CxCD3-biAb-1 administration in humans.

Three (3) escalating dose levels (0.03, 0.3, and 1.2 mg/kg) were tested with GUCY2CxCD3_biAb-3 to assess the toxic dose level of GUCY2CxCD3_biAb-1. Each dose was administered twice (1× weekly) over 2 weeks to 1 monkey per sex per group.

The results show that all doses were tolerated (HNSTD=1.2 mg/kg). No mortality or moribundity was observed. Clinical signs included transient emesis in the highest treated group (1.2 mg/kg) after the first dose (Day 1) only, where no intervention is needed, and no emesis was observed after the second dose administration (Day 8). The GUCY2CxCD3_biAb-3 (containing the GUCY2C binder of the application) showed less undesired side effects (such as one or more from among emesis, high body temperature, decreased activity, hunched posture, reduced appetite; dehydration, body weight loss (<7%), and soft or liquid feces) than the reference biAb.

### Example 6. Organoid Experiment

T cell-mediated cytotoxic activity of GUCY2CxCD3_biAb-1 was evaluated in tumor and WT intestinal human organoids. Organoids are in vitro 3D cultures derived from normal and diseased tissues. They faithfully mimic in vivo structure, functionality, and genetic features of original tissues. Because organoids retain the polarity of the original tissues, organoids could be utilized to probe the therapeutic hypothesis that GUCY2CxCD3_biAb-1 would differentially affect tumors, which are depolarized, compared to normal tissue where GUCY2C maintains polarity.

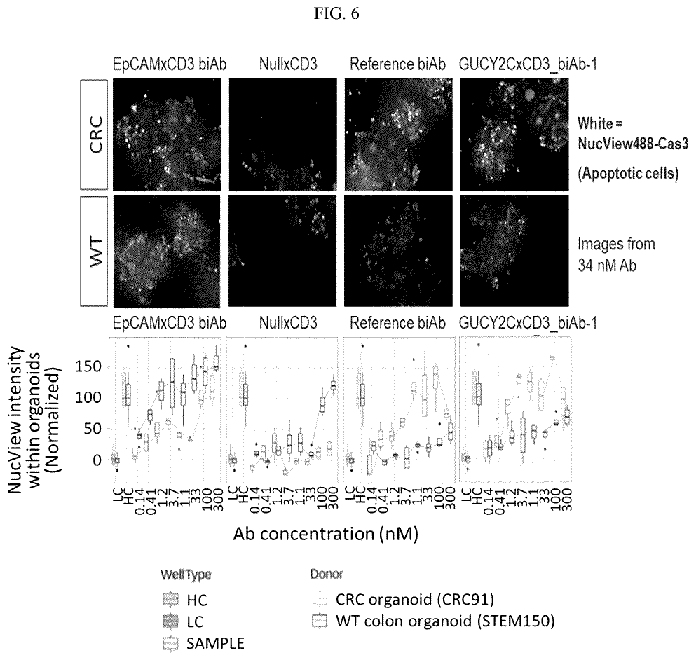

To evaluate T cell-mediated cytotoxic activity of GUCY2CxCD3_biAb-1 on WT and CRC tumor organoids, T cells and antibodies were incubated under normal culture condition (ie, with basal side facing outward) for 5 days. To measure T-cell-mediated cytotoxic activity, an apoptosis signal (ie, NucView488-Caspase3/7 dye) was detected within prestained organoids (ie, CellTracer-Yellow) using IncuCyte live imaging. T-cell activation against organoids were measured using flow cytometry. GUCY2CxCD3_biAb-1 induced dose-dependent apoptosis in CRC organoids and showed a clear selectivity window of cytotoxic activity between CRC and WT colon organoids, while a control EpCAMxCD3 biAb antibody (targeting EpCAM, a non-apically polarized tumor target) induced comparable robust cytotoxicity in both CRC and WT organoids (FIG. 6). Consistent with the high-content imaging result, evaluation of CD69, an early activation marker, on CD8⁺ T cells indicated a significant increase of CD69⁺ CD8⁺ cells in CRC organoids as compared to WT organoids in the presence of GUCY2CxCD3_biAb-1 (data not shown).

1. An isolated antibody or antigen binding fragment thereof comprising a first binding domain, wherein the first binding domain binds to guanylyl cyclase C (GUCY2C), and wherein the first binding domain comprises a first heavy chain complementarity determining region (HCDR) 1, a first HCDR2, and a first HCDR3 of a first heavy chain variable region (VH1) of SEQ ID NO: 7 and a first light chain complementarity determining region (LCDR) 1, a first LCDR2, and a first LCDR3 of a first light chain variable region (VL1) of SEQ ID NO: 8, wherein the HCDR1, HCDR2, HCDR3, LCDR1, LCDR2, and LCDR3 amino acid sequences are according to the Kabat numbering system, the Chothia numbering system, the AbM numbering system, the Contact numbering system or the IMGT numbering system or a combination thereof.

2. (canceled)

3. The isolated antibody or antigen binding fragment thereof of claim 1, wherein the first binding domain comprises the first HCDR1, the first HCDR2, the first HCDR3, the first LCDR1, the first LCDR2, and the first LCDR3 of
a. SEQ ID NOs: 1, 2, 3, 4, 5, 6, respectively;
b. SEQ ID NO: 23, 24, 3, 4, 5, and 6, respectively;
c. SEQ ID NO: 25, 26, 3, 4, 5, and 6, respectively; or
d. SEQ ID NO: 27, 28, 79, 29, 30 and 32, respectively.

4. The isolated antibody or antigen binding fragment thereof of claim 1, wherein the first binding domain comprises a heavy chain variable region having an amino acid sequence which is at least 90%, or at least 95%, or at least 98%, at least 99%, or 100% identical to SEQ ID NO: 7 and a light chain variable region having an amino acid sequence which is at least 90%, or at least 95%, or at least 98%, or at least 99% or 100% identical to SEQ ID NO: 8.

5. (canceled)

6. (canceled)

7. (canceled)

8. The isolated antibody or antigen binding fragment thereof of claim 1, wherein the first binding domain is or comprises a Fab.

9. The isolated antibody or antigen binding fragment thereof of claim 1, which is monospecific or multispecific.

10. (canceled)

11. (canceled)

12. (canceled)

13. The isolated antibody or antigen binding fragment thereof of claim 1, which further comprises a second binding domain that binds an antigen on a lymphocyte.

14. (canceled)

15. (canceled)

16. (canceled)

17. The isolated antibody or antigen binding fragment thereof of claim 13, wherein the antigen on the lymphocyte is CD3, CD8, BTNL3, CD186, BTNL8, PD-1, CD195, KI2L4, NKG2E, NKG2D, NKG2F, BTNL3, CD186, BTNL8, PD-1, or NKG2C.

18. (canceled)

19. (canceled)

20. (canceled)

21. (canceled)

22. The isolated antibody or antigen binding fragment thereof of claim 17, wherein the second binding domain binds to CD3 epsilon (CD3F) and comprises a second HCDR1, a second HCDR2, and a second HCDR3 of a second VH (VH2) of SEQ ID NO: 17 and a second LCDR1, a second LCDR2, and a second LCDR3 of a second VL (VL2) of SEQ ID NO: 18.

23. The isolated antibody or antigen binding fragment thereof of claim 22, wherein the second binding domain comprises the second HCDR1, the second HCDR2, the second HCDR3, the second LCDR1, the second LCDR2, and the second LCDR3 of SEQ ID NOs: 11, 12, 13, 14, 15, and 16, respectively.

24. The isolated antibody or antigen binding fragment thereof of claim 23, wherein the second binding domain comprises a heavy chain variable region having an amino acid sequence which is at least 90%, or at least 95%, or at least 98%, or at least 99%, or 100% identical to SEQ ID NO: 17, and a light chain variable region having an amino acid sequence which is at least 90%, or at least 95%, or at least 98%, or at least 99%, or 100% identical to SEQ ID NO: 18.

25. (canceled)

26. (canceled)

27. (canceled)

28. The isolated antibody or antigen binding fragment thereof of claim 22, wherein the second binding domain is a stapled scFv (spFv).

29. The isolated antibody or antigen binding fragment thereof of claim 28, wherein the spFv comprises a heavy chain variable region having the amino acid sequence of SEQ ID NO: 17 and a light chain variable region having the amino acid sequence of SEQ ID NO: 18.

30. (canceled)

31. (canceled)

32. (canceled)

33. The isolated antibody or antigen binding fragment thereof of claim 28, wherein the spFv comprises the sequence of SEQ ID NO: 22.

34. (canceled)

35. The isolated antibody or antigen binding fragment thereof of claim 13, which is of an IgG1 isotype.

36. (canceled)

37. (canceled)

38. The isolated antibody or antigen binding fragment thereof of claim 13, wherein first binding domain and/or the second binding domain is covalently connected to an IgG constant region, or a fragment of the IgG constant region, wherein the IgG constant region or the fragment of the IgG constant region comprises at least one of the following mutations:
i) at least one mutation that results in reduced binding of the antibody or antigen binding fragment thereof to a Fc receptor (FcγR), and the mutation comprises F234A/L235A, L234A/L235A, L234A/L235A/D265S, V234A/G237A/P238S/H268A/V309L/A330S/P331S, F234A/L235A, S228P/F234A/L235A, N297A, V234A/G237A, K214T/E233P/L234V/L235A/G236-deleted/A327G/P331A/D365E/L358M, H268Q/V309L/A330S/P331S, S267E/L328F, L234F/L235E/D265A, L234A/L235A/G237A/P238S/H268A/A330S/P331S, S228P/F234A/L235A/G237A/P238S, and S228P/F234A/L235A/G236-deleted/G237A/P238S;
(ii) at least one mutation that results in enhanced binding of the antibody or antigen binding fragment thereof to FcγR, and the mutation comprises S239D/I332E, S298A/E333A/K334A, F243L/R292P/Y300L, F243L/R292P/Y300L/P396L, F243L/R292P/Y300L/V305I/P396L, or G236A/S239D/I332E; and
(iii) at least one mutation that modulates a half-life of the antibody or antigen binding fragment thereof, and the mutation comprises H435A, P257I/N434H, D376V/N434H, M252Y/S254T/T256E/H433K/N434F, M252Y/S254T/T256E, T308P/N434A, or H435R; wherein residue numbering is according to the EU index.

39-45. (canceled)

46. The isolated antibody or antigen binding fragment thereof of claim 22, wherein the first binding domain is or comprises a first heavy chain (HC1) which comprises a Fab comprising a VH1 fused to a CH1, CH2, and CH3 of a human IgG1 heavy chain and a first light chain (LC1) comprising a VL1 fused to a C_{L} of human kappa light chain, and wherein the second binding domain comprises a second heavy chain (HC2) comprising a spFv in a “VL2-linker-VH2” orientation fused to a CH2 and CH3 of a human IgG1 heavy chain.

47. The isolated antibody or antigen binding fragment thereof of claim 46, wherein the HC1 comprises the T366S, L368A, and Y407V mutations and the HC2 comprises the T366W mutation (numbering is according to EU index); or wherein the HC1 comprises the T366W mutation and the HC2 comprises the T366S, L368A, and Y407V mutations (numbering is according to EU index).

48. The isolated antibody or antigen binding fragment thereof of claim 47, wherein the HC1 comprises an amino acid sequence at least 90%, at least 95%, at least 98%, at least 99% or 100% identical to SEQ ID NO: 9; the LC1 comprises an amino acid sequence at least 90%, at least 95%, at least 98%, at least 99% or 100% identical to SEQ ID NO: 10; and the HC2 comprises an amino acid sequence at least 90%, at least 95%, at least 98%, at least 99% or 100% identical to SEQ ID NO: 19.

49. (canceled)

50. The isolated antibody or antigen binding fragment thereof of claim 26, wherein the HC1 comprises the amino acid sequence of SEQ ID NO: 9, the LC1 comprises the amino acid sequence of SEQ ID NO: 10, and the HC2 comprises the amino acid of SEQ ID NO: 19.

51. (canceled)

52. (canceled)

53. (canceled)

54. A pharmaceutical composition comprising the isolated antibody or antigen binding fragment thereof of claim 1.

55. A polynucleotide encoding the isolated antibody or antigen binding fragment thereof of claim 1.

56. A vector comprising the polynucleotide of claim 55.

57. A host cell expressing the isolated antibody or antigen binding fragment thereof of claim 1.

58. (canceled)

59. (canceled)

60. A method of killing cancer cells overexpressing GUCY2C in a human subject, comprising administering the isolated antibody or antigen binding fragment thereof of claim 13 to the subject, thereby killing the cancer cells.

61. (canceled)

62. (canceled)

63. The method of claim 100, wherein the cancer cells are selected from colorectal cancer cells, gastric cancer cells, esophageal cancer cells and pancreatic cancer cells.

64. (canceled)

65. The method of claim 100, wherein the cancer cells are advanced or metastatic colorectal cancer cells, or unresectable metastatic colorectal cancer cells.

66-99. (canceled)

100. A method of treating a cancer in a human subject in need thereof, comprising administering the isolated antibody or antigen binding fragment thereof of claim 13 or the pharmaceutical composition thereof to the subject, thereby treating the cancer.
